(19) United States
(12) Patent Application Publication
(10) Pub. No.: US 2014/0255430 A1
(43) Pub. Date: Sep. 11, 2014

(54) METHODS AND REAGENTS FOR DETECTION AND TREATMENT OF ESOPHAGEAL METAPLASIA

(71) Applicants: NATIONAL UNIVERSITY OF SINGAPORE, Singapore (SG); Agency for Science, Technology and Research (A*Star), Singapore (SG); Harvard University, Cambridge, MA (US); Brigham & Women's Hospital, Boston, MA (US); Multiclonal Therapeutics, Inc., Newburyport, MA (US)

(72) Inventors: Wa XIAN, Singapore (SG); Frank MCKEON, Boston, MA (US); Matthew VINCENT, Newburyport, MA (US); Christopher CRUM, Boston, MA (US); Khek Yu HO, Singapore (SG)

(73) Assignees: NATIONAL UNIVERSITY OF SINGAPORE, Singapore (SG); Agency for Science, Technology and Research (A*Star), Singapore (SG); Harvard University, Cambridge, MA (US); Brigham & Women's Hospital, Boston, MA (US); Multiclonal Therapeutics, Inc., Newburyport, MA (US)

(21) Appl. No.: 14/136,736

(22) Filed: Dec. 20, 2013

Related U.S. Application Data
(63) Continuation of application No. 13/876,476, now abandoned.
(60) Provisional application No. 61/388,394, filed on Sep. 30, 2010.

(51) Int. Cl. C12N 5/095; G01N 33/574; C12Q 1/68; G01N 33/50

(52) U.S. Cl. CPC C12N 5/0695; G01N 33/5011; G01N 33/57407; C12Q 1/6886
USPC 424/174.1; 435/325; 435/29; 435/7.23; 435/6.1; 506/9

(57) ABSTRACT

The invention described herein relates to the treatment, detection, and diagnosis of various cancers, including esophageal or gastric adenocarcinoma and related metaplasias. The invention also includes a clonal population of Barrett's esophagus progenitor cells and methods of using them for the treatment, detection, and diagnosis of Barrett's esophagus.

### RELATED APPLICATION

This application is a continuation of U.S. application Ser. No. 13/876,476 filed Mar. 27, 2013, Attorney Docket No. 544332 ET9-001 US, which is an 35 U.S.C. §371 filing of International Application No. PCT/US2011/054323, filed Sep. 30, 2011, which claims priority to U.S. Provisional Application No. 61/388,394, Attorney Docket No. ET9-001-1, filed Sep. 30, 2010, entitled “METHODS AND REAGENTS FOR DETECTION AND TREATMENT OF ESOPHAGEAL METAPLASIA”. The contents of any patents, patent applications, and references cited throughout this specification are hereby incorporated by reference in their entireties.

### GOVERNMENTAL FUNDING

The invention described herein was supported, in part, by grants from the National Institutes of Health (R01 GM 083348). The United States government may have certain rights in the invention.

### FIELD OF THE INVENTION

The invention described herein relates to the treatment, detection, and diagnosis of various cancers, including esophageal or gastric adenocarcinoma and related metaplasias.

### BACKGROUND OF THE INVENTION

Esophageal and gastric adenocarcinoma together kill more than one million people each year worldwide and represent the 2nd leading cause of death from cancer. Both cancers arise in association with chronic inflammation and are preceded by robust metaplasia with intestinal characteristics. In fact, the patient population with precancerous lesions is estimated to be significantly larger—in the range of 100 million people in size—all at substantial risk of developing cancer in their lifetimes. Current treatments for both cancer and precancerous patients have an exceptionally high degree of relapse, with the 5 year survival rate for patients developing cancer being marginal.

Gastric intestinal metaplasia can be triggered by gastritis involving H. pylori infections, while Barrett's metaplasia of the esophagus is linked to gastroesophageal reflux disease (GERD). While H. pylori suppression therapies have contributed to the recent decline of gastric adenocarcinoma, the incidence of esophageal adenocarcinoma, especially in the West, has increased dramatically in the past several decades (Spechler et al. N Engl J. Med. 1986; 315:362-71; Blot et al. JAMA 1991; 265:1287-9; Raskin et al. Cancer Res 1992; 52:2946-50; Jankowski et al. Am J Pathol 1999; 154:965-973; and Reid et al. Nat Rev Cancer 2010; 10:87-101). Treatments for late stages of these diseases are challenging and largely palliative, and therefore considerable efforts have focused on understanding the earlier, premalignant stages of these diseases for therapeutic opportunities.

The prevailing theory for the development of metaplasia has been that the abnormal cells seen in Barrett's esophagus arise as the normal squamous cells “transcommit” in response to inflammation (such as acid-reflux) to a new, intestine-like fate. Intestine-like metaplasia is a columnar epithelium marked by prominent goblet cells and intestinal markers such as villin and trefoil factors 1, 2, and 3, and, once established, appears to be irreversible (Sagar et al. Br J Surg. 1995; 82:806-10; Barr et al. Lancet 1996; 348:584-5; and Watari et al. Clin Gastroenterol Hepatol 2008; 6:409-17). There is compelling evidence for a dynamic competition among clones of cells within Barrett's metaplasia that almost certainly contributes to its premalignant progression. Cancers arise from this metaplasia via stereotypic genetic and cytologic changes that present as dysplasia, high-grade dysplasia, and finally invasive adenocarcinoma (Raskin et al., supra; Jankowski et al., supra; Haggitt. Hum Pathol 1994; 25:982-93; Schlemper et al. Gut 2000; 47:251-5; and Correa et al. Am J Gastroenterol 2010; 105:493-8).

### SUMMARY OF THE INVENTION

An understanding of the ontogeny of gastric intestinal metaplasia would allow for the development of compositions and methods for the early detection and treatment of gastric intestinal metaplasia prior to progression to adenocarcinoma. As described in greater detail herein, the inventors have replaced the old paradigm of transcommittment of cell fate with a new understanding of the origins of esophageal and gastric metaplasias in which stem cells of embryonic origin—left behind during organogenesis of the alimentary canal—give rise to the precancerous diseases and ultimately to esophageal and gastric adenocarcinoma. The inventors have shown that this discrete population stem cells persist in humans at the squamocolumnar junction, the source of Barrett's metaplasia. The inventors have also shown that upon damage to the squamous epithelium, these stem cell are activated and proliferate in the development of the precancerous lesions. The findings presented in this application demonstrate that gastric intestinal and Barrett's metaplasias initiate not from genetic alterations or transcommittment of differentiated tissue, but rather from competitive interactions between cell lineages driven by opportunity. Targeting these precancerous lesions by preventing growth and/or differentiation of these vestigial stem cells, which have proven to be resistant to physical ablation and other therapies directed to the resulting metaplasias, offers a unique opportunity to prevent progression to cancer in a very large patient population.

As described in further detail in this application, the inventors have isolated these cancer stem cells, as well as normal epithelial stem cells for the esophagus, stomach and intestines, and through gene expression profiling have identified a number of targets for development of antibodies, RNAi and small therapeutics that may be selectively lethal to the cancer stem cell relative to rest of the alimentary canal. With the isolated cells in hand, there is not the opportunity to rapidly develop drug candidates with selectivity and in vitro efficacy. Coupled with animal models for these diseases presented herein and others available in the art, there is a clear preclinical and clinical path to providing effective therapies. While it is expected that systemic delivery of therapeutic agents is an option, the fact of the matter is that the sites of treatment lend themselves well to oral or endoscopic depot delivery. The dim prognosis for gastric intestinal and esophageal adenocarcinoma argues for therapies directed at preventing even the initiation of the precancerous metaplasia. For these precancerous metaplasia patients again numbering in the tens of millions—this provides a ten to twenty year window for treatment before cancer would typically develop.

Accordingly, a salient feature to the current application is the discovery that a unique population of primitive epithelial stem cells give rise to the metaplasia underlying esophageal and gastric adenocarcinoma and that these primitive epithelial stem cells have a distinct molecular signature that can be exploited for diagnostic and therapeutic targeting. For instance, these discoveries allow for the therapeutic targeting of the population of stem cells responsible for the metaplasia using cytotoxic and/or growth inhibitory and/or differentiation inhibitory agents, particularly agents selective for the stem cell relative to normal squamous cells or regenerative stem cells of the esophagus or stomach, thus facilitating the treatment of metaplasia and prevention of its progression to adenocarcinoma. Likewise, the use of agents directed to gene products unique to the stem cell, particularly cell surface markers that can be detected with antibodies, the present invention provides reagents and methods for detecting the stem cell in tissue biopsy samples as well as in vivo (i.e., for imaging or detection using endoscopic visualization). Given the accessibility of these tissues through non-invasive and minimally invasive techniques, in certain preferred embodiments the therapeutic agents or imaging agents are delivered by direct injection, such as by endoscopic injection.

The following are merely illustrative. In the case of a gene encoding a cell surface protein, the therapeutic agent can be an antibody or antibody mimetic, i.e., one which inhibits growth or differentiation by inhibiting the function of the cell surface protein, or one which is cytotoxic to the cell as a consequence to invoking an immunological response (i.e., ADCC) against the targeted stem cell. In the case of a gene encoding an enzyme, the therapeutic may be a small molecule inhibitor of the enzymatic activity, or a prodrug including a substrate for the enzyme such that the prodrug is converted to an activate agent upon cleavage of the substrate portion. In the case of transcription factors, the therapeutic agent may be a decoy nucleic acid that competes with the genomic regulatory elements for binding to the transcription factor; or in the case of ligand-mediated transcription factors (such as PPARγ), may be an agonist or antagonist ligand of the transcription factor. In instances where the viability, growth or differentiation of the target stem cell is dependent on the level of expression of the gene, then use of antisense, RNAi or other inhibitory nucleic acid therapeutics can be considered.

In one aspect, the invention provides a method for treating or preventing esophageal metaplasia, comprising administering to a subject a therapeutic amount of an agent that decreases the expression and/or biological activity of one or more of the genes set forth in Tables 1-5 and FIGS. 9-11, such that the metaplasia is treated or prevented. In certain embodiments, the agent is an antibody, antibody-like molecule, antisense oligonucleotide, small molecule or RNAi agent.

In another aspect, the invention provides a method for treating or preventing esophageal metaplasia, comprising administering a therapeutic amount of an agent that specifically binds to a cell surface polypeptide encoded by one of the genes set forth in Tables 1-5 and FIGS. 9-11, wherein said agent is linked to one or more cytotoxic moiety. In certain embodiments, the agent is an antibody, antibody-like molecule or cell surface receptor ligand. The cytotoxic moiety can be, for example, a radioactive isotope, chemotoxin, or toxin protein. In certain embodiments, the cytotoxic moiety is encapsulated in a biocompatible delivery vehicle including, without limitation, microcapsules, microparticles, nanoparticles, and liposomes. In some embodiments, the agent is directly linked to the cytotoxic moiety.

In another aspect, the invention provides a method of imaging esophageal metaplasia, the method comprising administering to a subject an effective amount of an agent that specifically binds to a cell surface polypeptide encoded by one of the genes set forth in Tables 1-5 and FIGS. 9-11, and visualizing the agent. In certain embodiments, the agent is an antibody, antibody-like molecule or cell surface receptor ligand. In certain embodiments, the agent is linked to an imaging moiety. The imaging moiety can be, for example, a positron-emitter, nuclear magnetic resonance spin probe, an optically visible dye, or an optically visible particle. The imaging agent may be one which permits non-invasive imaging, such as by MRI, PET or the like. In other embodiments, the imaging moiety can be a fluorescent probe or other optically active probe which can be visualized, e.g., through an endoscope.

According to the methods of the invention, a therapeutic and/or imaging agent can be administered by any suitable route and/or means including, without limitation, orally and/or parenterally. In a preferred embodiment, the agent is administered endoscopically to the esophageal squamocolumnar junction or a site of esophageal metaplasia.

In another aspect, the invention provides a method of detecting the presence or absence of the target stem cell in a tissue biopsy. Such detection agents can include antibodies and nucleic acids which bind to a gene or gene product unique to the stem cell relative to other normal or diseased esophageal tissue.

In another aspect, the invention provides a method of diagnosing, or predicting the future development or risk of development of, esophageal metaplasia or adenocarcinoma, comprising measuring the expression level of one or more of the genes set forth in Tables 1-5 and FIGS. 9-11 in an epithelial tissue sample from a subject, wherein an increase in the expression level relative to a suitable control indicates that the subject has, or has a future risk of developing, metaplasia. In some embodiments, mRNA levels of the gene are measured. In other embodiments, the levels of the protein product of the gene are measured. Such methods can be performed in vivo or in vitro.

In another aspect, the invention provides a method of identifying a compound useful for treating or preventing esophageal metaplasia, the method comprising administering a test compound to p63 null mouse and determining the amount of epithelial metaplasia in the presence and absence of the test compound, wherein a decrease in the amount of epithelial metaplasia identifies a compound useful for treating esophageal metaplasia.

In another aspect, the invention provides a method of identifying a compound useful for treating or preventing esophageal metaplasia, the method comprising administering a test compound to a mouse, wherein the mouse comprises stratified epithelial tissue in which basal cells have been ablated, and determining the amount of epithelial metaplasia in said epithelial tissue in the presence and absence of the test compound, wherein a decrease in the amount of epithelial metaplasia identifies a compound useful for treating esophageal metaplasia.

The invention further provides a composition comprising a clonal population of Barrett's Esophagus (BE) stem cells, such as may be isolated from an esophagus of a subject or generated from ES cells or iPS cells, wherein the stem cells differentiate into Barrett's epithelium (i.e., columnar epithelium). Preferably the composition, with respect to the cellular component, is at least 50 percent BE stem cell, more preferably at least 75, 80, 85, 90, 95 or even 99 percent BE stem cell. The BE stem cells can be pluripotent, multipotent or oligopotent. In certain preferred embodiments, the BE stem cells are characterized as having an mRNA profile can further include a profile wherein the amount of one or more of GSTM4, SLC16A4, CMBL, CEACAM6, NRFA2, CFTR, GCNT3 mRNA in the clonal cell population are each in the range of 5 to 50 percent of the amount of actin mRNA in the clonal cell population, more preferably in the range of 10-25 percent. Preferably all seven genes have an mRNA profile in that range. In certain embodiments, the mRNA transcript profile for the BE cells will also be characterized by detectable levels of BICC1 and NTS. In certain embodiments, the BE cells will also be characterized by non-detectable levels of SOX2, p63, Krt20, GKN1/2, FABP1/2, Krt14, CXCL17, i.e., less than 0.1 percent the level of actin, and even more preferably less than 0.01 or even 0.001 percent the level of actin mRNA.

In an additional embodiment, the BE stem cells are characterized as CEACAM6 positive, and Krt20, Sox2 and p63 negative, as detected by standard antibody staining. For instance, levels of Krt20, Sox2 and p63 are less than 10 percent of the level of CEACAM6, and more preferably less than 5 percent, 1 percent, and even less than 0.1 percent.

The invention further provides a composition comprising a population of cells enriched in a clonal subpopulation of BE stem cells from an esophagus of a subject, wherein the clonal subpopulation of cells differentiates into Barrett's epithelium (i.e., columnar epithelium). The BE stem cells can be pluripotent, multipotent or oligopotent.

Another aspect of the invention provides a clonal population of Barrett's Esophagus (BE) stem cells, derived from human or stem cell or iPS cell sources, characterized as having an mRNA profile can further include a profile wherein the amount of one or more of GSTM4, SLC16A4, CMBL, CEACAM6, NRFA2, CFTR, GCNT3 mRNA in the stem cell population are each in the range of 5 to 50 percent of the amount of actin mRNA in the clonal cell population, more preferably in the range of 10-25 percent. Preferably all seven genes have an mRNA profile in that range. In certain embodiments, the mRNA transcript profile for the BE cells will also be characterized by detectable levels of BICC1 and NTS. In certain embodiments, the BE cells will also be characterized by non-detectable levels of SOX2, p63, Krt20, GKN1/2, FABP1/2, Krt14, CXCL17, i.e., less than 0.1 percent the level of actin, and even more preferably less than 0.01 or even 0.001 percent the level of actin mRNA. The clonal population of BE stem cells may also be characterized as CEACAM6 positive, and Krt20, Sox2 and p63 negative, as detected by standard antibody staining. For instance, levels of Krt20, Sox2 and p63 are less than 10 percent of the level of CEACAM6, and more preferably less than 5 percent, 1 percent, and even less than 0.1 percent.

The invention further provides a method of screening for an agent effective in the treatment or prevention of Barrett's esophagus including the steps of providing a population of BE stem cells, wherein the BE stem cells are able to differentiate into Barrett's epithelium; providing a test agent; and exposing the BE stem cells to the test agent; wherein if the test agent is cytotoxic, cytostatic and/or able to inhibit the differentiation of the BE stem cells to columnar epithelial cells, the test agent is an agent effective in the treatment or prevention of Barrett's esophagus.

In certain embodiments, the BE stem cells are mammalian BE stem cells, such as human BE stem cells.

In certain embodiments, candidate therapeutic agents reduce the viability, growth or ability to differentiation by 70, 80, 90, 95, 96, 97, 98, 99 or even 100%.

The BE stem cells can be clonal, and can be pluripotent, multipotent or oligopotent. In certain preferred embodiments, the BE stem cells are characterized as having an mRNA profile can further include a profile wherein the amount of one or more of GSTM4, SLC16A4, CMBL, CEACAM6, NRFA2, CFTR, GCNT3 mRNA in the stem cell population are each in the range of 5 to 50 percent of the amount of actin mRNA in the stem cell population, more preferably in the range of 10-25 percent. Preferably all seven genes have an mRNA profile in that range. In certain embodiments, the mRNA transcript profile for the BE cells will also be characterized by detectable levels of BICC1 and NTS. In certain embodiments, the BE cells will also be characterized by non-detectable levels of SOX2, p63, Krt20, GKN1/2, FABP1/2, Krt14, CXCL17, i.e., less than 0.1 percent the level of actin, and even more preferably less than 0.01 or even 0.001 percent the level of actin mRNA. The clonal population of BE stem cells may also be characterized as CEACAM6 positive, and Krt20, Sox2 and p63 negative, as detected by standard antibody staining. For instance, levels of Krt20, Sox2 and p63 are less than 10 percent of the level of CEACAM6, and more preferably less than 5 percent, 1 percent, and even less than 0.1 percent.

The invention further provides a method of screening for an agent effective in the detection of Barrett's esophagus including the steps of providing BE stem cells; providing a test agent; and exposing the BE stem cells to the test agent; wherein if the test agent specifically binds to the BE stem cells, i.e., relative to normal squamous cells or intestinal cells or Barrett's epithelial cells, the test agent is an agent effective in the detection of stem cells giving rise to Barrett's esophagus.

In certain embodiments, the BE stem cells are mammalian, and more preferably are human.

In certain embodiments, the test agent specifically binds to a cell surface protein on the stem cells. Cell surface proteins include CEACAM6, MMP1, SLC26A3, TSPAN8, LYZ and SPINK1. Specifically, the test agent can be an antibody. Optionally, the antibody can be a monoclonal antibody.

The invention further provides a method of detecting the presence of Barrett's esophagus in a subject including the steps of providing a detection agent that specifically binds to BE stem cells; administering the detection agent to a subject; and detecting whether the detection agent specifically binds to a BE stem cell in the esophagus of the subject, wherein, if the detection agent specifically binds to a cell in the esophagus of the subject to a higher degree than the average non-Barrett's esophagus patient, the subject is diagnosed with Barrett's esophagus or as having a risk of developing Barrett's esophagus.

The invention further provides a method of for treating or preventing Barrett's esophagus and/or esophageal metaplasia in a subject in need thereof comprising administering to subject an effective amount of an agent that is cytotoxic or cytostatic for Barrett's Esophagus stem cells in the esophagus of the subject, or inhibits differentiation of the Barrett's Esophagus stem cells to columnar epithelium.

In certain embodiments, the subject is a mammal. In a preferred embodiment, the mammal is human.

In certain embodiments, candidate therapeutic agents reduce the viability, growth or ability to differentiation by 70, 80, 90, 95, 96, 97, 98, 99 or even 100%.

The targeted BE stem cells can characterized as having an mRNA profile that can further include a profile wherein the amount of one or more of GSTM4, SLC16A4, CMBL, CEACAM6, NRFA2, CFTR, GCNT3 mRNA in the stem cell population are each in the range of 5 to 50 percent of the amount of actin mRNA in the stem cell population, more preferably in the range of 10-25 percent. Preferably all seven genes have an mRNA profile in that range. In certain embodiments, the mRNA transcript profile for the BE cells will also be characterized by detectable levels of BICC1 and NTS. In certain embodiments, the BE cells will also be characterized by non-detectable levels of SOX2, p63, Krt20, GKN1/2, FABP1/2, Krt14, CXCL17, i.e., less than 0.1 percent the level of actin, and even more preferably less than 0.01 or even 0.001 percent the level of actin mRNA. The stem population of BE stem cells may also be characterized as CEACAM6 positive, and Krt20, Sox2 and p63 negative, as detected by standard antibody staining. For instance, levels of Krt20, Sox2 and p63 are less than 10 percent of the level of CEACAM6, and more preferably less than 5 percent, 1 percent, and even less than 0.1 percent.

In certain embodiments, the therapeutic agent specifically binds to a cell surface protein on the BE stem cells. Cell surface proteins include CEACAM6, MMP1, SLC26A3, TSPAN8, LYZ and SPINK1. Specifically, the therapeutic agent can be an antibody. Optionally, the antibody can be a monoclonal antibody. The antibody can be conjugated to a cytotoxic or cytostatic moiety.

The therapeutic agent can be selected from the group consisting of produgs comprising a medoximil moiety, PPARγ inhibitors and NR5A2 activity modulators. The test agent can also be an RNAi or antisense composition. The RNAi or antisense composition can reduce the amount of mRNA in the targeted BE stem cells of a member of the group consisting of GSTM4, SLC16A4, CMBL, CEACAM6, NR5A2, CFTR, GCNT3 and PPARγ.

The invention further provides a composition comprising a population of squamous stem cells isolated from an esophagus of a subject, wherein the squamous stem cells differentiate into normal squamous epithelial cells of the esophagus, i.e., the squamous stem cells are regenerative. The squamous stem cells can be clonal, and can be pluripotent, multipotent or oligopotent. In certain preferred embodiments, the squamous stem cells are characterized as having an mRNA profile can further include a profile wherein the amount of one or more of S100A8, Krt14, SPRR1A or CSTA mRNA in the stem cell population are each in the range of 5 to 50 percent of the amount of actin mRNA in the stem cell population, more preferably in the range of 10-25 percent. Preferably all seven genes have an mRNA profile in that range. In certain embodiments, the squamous cells will also be characterized by non-detectable levels of SOX2, Krt20, CXCL17, CEACAM6 or NR5A2, i.e., less than 0.1 percent the level of actin, and even more preferably less than 0.01 or even 0.001 percent the level of actin mRNA. The clonal population of squamous stem cells may also be characterized as p63 positive, and CEACAM6 negative, as detected by standard antibody staining. For instance, levels of CEACAM6 are less than 10 percent of the level of p63, and more preferably less than 5 percent, 1 percent, and even less than 0.1 percent.

The invention further provides a composition comprising a clonal population of gastric cardia (GC) stem cells isolated from gastric cardia or esophagus of a subject, wherein the GC stem cells differentiates into gastric cardia cells of the stomach. The gastric cardia stem cells can be clonal, and can be pluripotent, multipotent or oligopotent. In certain preferred embodiments, the gastric cardia stem cells are characterized as having an mRNA profile can further include a profile wherein the amount of one or more of CXCL17, CAPN6, PSCA, GKN1, GKN2 or MT1G mRNA in the stem cell population are each in the range of 5 to 50 percent of the amount of actin mRNA in the stem cell population, more preferably in the range of 10-25 percent. Preferably all seven genes have an mRNA profile in that range. In certain embodiments, the gastric cardia cells will also be characterized by non-detectable levels of CEACAM6, p63, FABP1, FABP2, Krt14 or Krt20, i.e., less than 0.1 percent the level of actin, and even more preferably less than 0.01 or even 0.001 percent the level of actin mRNA. The clonal population of gastric cardia stem cells may also be characterized as CEACAM6 negative, as detected by standard antibody staining. For instance, levels of CEACAM6 are less than 10 percent of the level of CXCL17, and more preferably less than 5 percent, 1 percent, and even less than 0.1 percent.

### BRIEF DESCRIPTION OF THE FIGURES

FIG. 1. Metaplasia in the Proximal Stomach of p63 Null Embryos. Panel A shows a section through the stomach of an E18 wild type mouse highlighting the p63-positive squamous epithelia of the proximal stomach (PS) and the glandular epithelium of the distal stomach (DS). Panel B are immunofluorescence images of E17 wild type (WT) and p63 null (KO) sections of epidermis showing the intermittent staining for basal (anti-keratin 5) and differentiated (anti-loricrin) markers reflecting the degradation of the p63 null epidermis due to loss of epidermal stem cells. Panel C shows a comparison of H&E stained sections through stomachs of E18 wild type and p63 null embryos.

FIG. 2. Gene Expression of Metaplasia in p63 Null Embryos. Panels A and B show Principle Component Analysis and heat maps of expression microarray data comparisons between E18 wild type (WT) and p63 null (KO) proximal stomachs and other indicated gastrointestinal tissues from these embryos. PS, proximal stomach; DS, distal stomach; LI, large intestine; SI, small intestine. “Intestine-like” box are genes in common with lower portions of the gastrointestinal tract; “Unique” box contains genes specific to the metaplasia. Panel C shows gene expression heat maps comparing genes high and low in wild type and p63 null proximal stomach and compares these to gene expression patterns preformed on datasets comparing normal human esophagus and Barrett's metaplasia. Panels D and E show the relative expression of known Barrett's metaplasia biomarkers in the metaplasia of the E18 p63 null embryos compared to wild type proximal stomach (p<10⁻⁷ for all), and the validation of several markers by immunohistochemistry on sections of wild type and mutant proximal stomach.

FIG. 3. Retrospective Tracing of Metaplasia through Embryogenesis. Panel A shows a series of immunofluorescence images using antibodies against claudin 3 (Cnd3), keratin 7 (Krt7), and Car4/Cnd3 on sections of E18 metaplasia in p63 null embryos. These markers were used to track the metaplasia back through timed embryos to E14, where the metaplasia labels with Car4, Krt7, and is highly proliferative as judged by Ki67 staining in Panel B. Panel C shows that one day earlier, at E13, both wild type and p63 null proximal stomachs display a similar layer of Car-4-positive cells in the proximal stomach. Panel D shows sections though wild type E13 (left) and E14 (right) proximal stomachs probed with antibodies to Car4 and p63. Arrow depicts an anterior-to-posterior gradient of p63 positive cells from esophagus to proximal stomach.

FIG. 4. Persistence of Embryonic Epithelium at the Squamocolumnar Junction. Panels A-C show the distribution of the keratin 7 (Krt7, green)-expressing cells in wild type embryos from its suprasquamous position at E17, its disintegration at E18, and its remnant population residing at the squamocolumnar junction of the stomach in E19 embryos and three-week-old mice. The basal cells of the squamous epithelium of the proximal stomach are labeled with antibodies to keratin 5 (Krt5, red). Panel E shows a gene expression analysis of the residual embryonic epithelium of three-week-old mice.

FIG. 5. Upregulation of Muc4 in epithelium at the Squamocolumnar Junction, Panel A shows immunofluorescence images using antibodies against Muc4. Panel B depicts a schematic for the ontogeny of Barrett's metaplasia from residual embryonic cells at the squamocolumnar junction in response to epithelial damage.

FIG. 6. Histological Analysis of Car-4-Expressing and p63-Expressing Cells During the Development of the Squamocolumnar Junction in Mice.

FIG. 7. Histological Analysis of the Squamocolumnar Junction in Wild-type (Panel A) and p63 Null Mice (Panel B) at E17 to E19.

FIG. 8. Histological Analysis of Squamocolumnar Junctional Markers identified by Gene Expression Profiling in Wild-type and p63 Null Mice at E18.

FIG. 9. Novel Biomarkers of Barrett's Metaplasia Identified by Gene Expression Profiling of Barrett's-like Metaplasia in the p63 null mice

FIG. 10. Cell Surface Markers Genes of Barrett's Metaplasia Identified by Gene Expression Profiling of Barrett's-like Metaplasia in the p63 null mice.

FIG. 11. Genes Upregulated in both the cells of Squamocolumnar Junction of the Stomach and in the Barrett's-like Metaplasia in the p63 null mice.

FIG. 12. Gene Expression of Barrett's Esophagus Progenitor Cells Compared to Gene Expression in Squamous Cell Progenitor Cells.

FIG. 13. Protein expression in Barrett's Esophagus Progenitor Cells Compared to Protein Expression in Squamous and Gastric Cardia Progenitor Cells.

FIG. 14. Protein expression in Barrett's Esophagus Progenitor Cells Compared to Protein Expression in Gastric Cardia Progenitor Cells.

FIG. 15 is a schematic showing ligands of NR5A2.

### DETAILED DESCRIPTION OF INVENTION

### I. Overview

The present invention is based, in part, on the discovery that a unique population of primitive epithelial cells give rise to the metaplasia underlying esophageal and gastric adenocarcinoma and that these cells have a distinct molecular signature.

Specifically, Applicants have demonstrated that during murine embryogenesis, squamous stem cells displace a primitive epithelium in the proximal stomach from the basement membrane to a proliferatively dormant, suprasquamous position. However, in mice lacking p63 (a protein that is essential for the self-renewal of stem cells of all stratified epithelial tissues, including mammary and prostate glands as well as all squamous epithelial), these squamous stem cells fail to supplant the primitive epithelium, which then rapidly emerges into a columnar metaplasia with gene expression profiles similar to Barrett's metaplasia but unique to the gastrointestinal tract. Moreover, in adults, a discrete population of these primitive epithelial cells survives embryonic development and resides at the squamocolumnar junction. Upon diptheria toxin-mediated ablation of squamous epithelial stem cells, these residual embryonic cells begin to invade vacated regions of basement membrane originating a highly proliferative metaplasia. Applicants have further performed histological and gene expression analyses of the metaplasia evident in mouse models of extreme GERD during embryogenesis and in adults to assemble a relative genetic signature of these metaplasias and to define the mechanism of their evolution.

Applicants have also isolated a human Barrett's esophagus progenitor cell. This progenitor cell differentiates into Barrett's esophagus tissue and has a unique mRNA expression profile described below. Together, the clonal population of this Barrett's esophageal progenitor cell allows for the detection and direct therapeutic targeting of the population of cells responsible for the metaplasia by cytotoxic or and/or growth inhibitory agents, thus facilitating the treatment of metaplasia and prevention of its progression to adenocarcinoma. This human Barrett's esophagus progenitor cell can be isolated from human Barrett's metaplasia tissue by dissociating the cells in the tissue and isolating the progenitor cells via FACS using any of the cell surface proteins described in Table YY, below.

Applicants have also isolated human squamous cell and gastric cardia progenitor cells. Applicants have characterized the mRNA and protein expression of these cells to define these cells and to differentiate their expression profiles from Barrett's esophagus progenitor cells. This allows for the ablation of Barrett's esophagus progenitor cells without reducing the viability of nearby squamous cell or gastric cardia progenitor cells.

Accordingly, the present invention provides methods and compositions for diagnosing, imaging, treating or preventing metaplasia (e.g., esophageal metaplasia). The present invention also provides methods identifying compounds useful for treating esophageal metaplasia.

### II. Definitions

The term “agent” includes any substance, molecule, element, compound, entity, or a combination thereof. It includes, but is not limited to, e.g., protein, oligopeptide, small organic molecule, polysaccharide, polynucleotide, and the like. It can be a natural product, a synthetic compound, or a chemical compound, or a combination of two or more substances

As used herein, the term “RNAi agent” refers to an agent, such as a nucleic acid molecule, that mediates gene-silencing by RNA interference, including, without limitation, small interfering siRNAs, small hairpin RNA (shRNA), and microRNA (miRNA).

The term “cell surface receptor ligand”, as used herein, refers to any natural ligand for a cell surface receptor.

The term “antibody” encompasses any antibody (both polyclonal and monoclonal), or fragment thereof, from any animal species. Suitable antibody fragments include, without limitation, single chain antibodies (see e.g., Bird et al. (1988) Science 242:423-426; and Huston et al. (1988) Proc. Natl. Acad. Sci. U.S.A 85:5879-5883, each of which is herein incorporated by reference in its entirety), domain antibodies (see, e.g., U.S. Pat. Nos. 6,291,158; 6,582,915; 6,593,081; 6,172,197; 6,696,245, each of which is herein incorporated by reference in its entirety), Nanobodies (see, e.g., U.S. Pat. No. 6,765,087, which is herein incorporated by reference in its entirety), and UniBodies (see, e.g., WO2007/059782, which is herein incorporated by reference in its entirety

The term “antibody-like molecule”, as used herein, refers to a non-immunoglobulin protein that has been engineered to bind to a desired antigen. Examples of antibody-like molecules include, without limitation, Adnectins (see, e.g., WO 2009/083804, which is herein incorporated by reference in its entirety), Affibodies (see, e.g., U.S. Pat. No. 5,831,012, which is herein incorporated by reference in its entirety), DARPins (see, e.g., U.S. Patent Application Publication No. 2004/0132028, which is herein incorporated by reference in its entirety), Anticalins (see, e.g., U.S. Pat. No. 7,250,297, which is herein incorporated by reference in its entirety), Avimers (see, e.g., U.S. Patent Application Publication Nos. 200610286603, which is herein incorporated by reference in its entirety), and Versabodies (see, e.g., U.S. Patent Application Publication No. 2007/0191272, which is hereby incorporated by reference in its entirety).

The term “cytotoxic moiety”, as used herein, refers to any agent that is detrimental to (e.g., kills) cells.

The term “chemotoxin”, as used herein, refers to any small molecule cytotoxic moiety that is detrimental to (e.g., kills) cells.

The term “biological activity” of a gene, as used herein, refers to a functional activity of the gene or its protein product in a biological system, e.g., enzymatic activity and transcriptional activity.

The term “p63 null mouse”, as used herein, refers to a mouse in which the p63 gene (NCBI Reference Sequence: NM_{—}011641.2) has been deleted or downregulated in one or more tissue (e.g., epithelial tissue).

The term “biocompatible delivery vehicle”, as used herein, refers to any phyioslogically compatible compound that can carry a drug payload, including, without limitation, microcapsules, microparticles, nanoparticles, and liposomes.

The term “imaging moiety”, as used herein, refers to an agent that can be detected and used to image tissue in vivo.

The term “ablated” or “ablation”, as used herein, refers to the functional removal of cells, e.g., the basal cells of the mouse stratified epithelial tissue, using any art-recognized means. In one embodiment, cells are ablated by treatment with a cytotoxic moiety, e.g., using Cre-mediated expression of diphtheria toxin fragment A as described in Ivanova et al. Genesis. 2005; 43:129-35. In other embodiments, cells are chemically or physically ablated, e.g., by endoscopy-assisted ablation, radiofrequency ablation, laser ablation, microwave ablation, cryogenic ablation, thermal ablation, chemical ablation, and the like. In one exemplary embodiment, the ablation energy is radio frequency electrical current applied to conductive needle. The electrical current may be selected to provide pulsed or sinusoidal waveforms, cutting waves, or blended waveforms. In addition, the electrical current may include ablation current followed by current sufficient to cauterize any blood vessels that may be compromised during the ablation process. Alternatively, in some embodiments, ablation probe may take the form of a bipolar probe that carries two or more electrodes, in which case the current flows between the electrodes.

The term “suitable control”, as used herein, refers to a measured mRNA or protein level (e.g. from a tissue sample not subject to treatment by an agent), or a reference value that has previously been established.

The term “pluripotent” as used herein, refers to a stem or progenitor cell that is capable of differentiating into any of the three germ layers endoderm, mesoderm or ectoderm.

The term “multipotent”, as used herein, refers to a stem or progenitor cell that is capable of differentiating into multiple lineages, but not all lineages. Often, multipotent cells can differentiate into most of the cells of a particular lineage, for example, hematopoietic stem cells.

The term “oligopotent”, as used herein, refers to a stem or progenitor cell that can differentiate into two to five cell types, for example, lymphoid or myeloid stem cells.

The term “positive”, as used herein, refers to the expression of an mRNA or protein in a cell, wherein the expression is at least 5 percent of the expression of actin in the cell.

The term “negative”, as used herein, refers to the expression of an mRNA or protein in a cell, wherein the expression is less than 1 percent of the expression of actin in the cell.

### III. Exemplary Embodiments

### A. Molecular Signature of Cells Responsible for the Esophageal Metaplasia

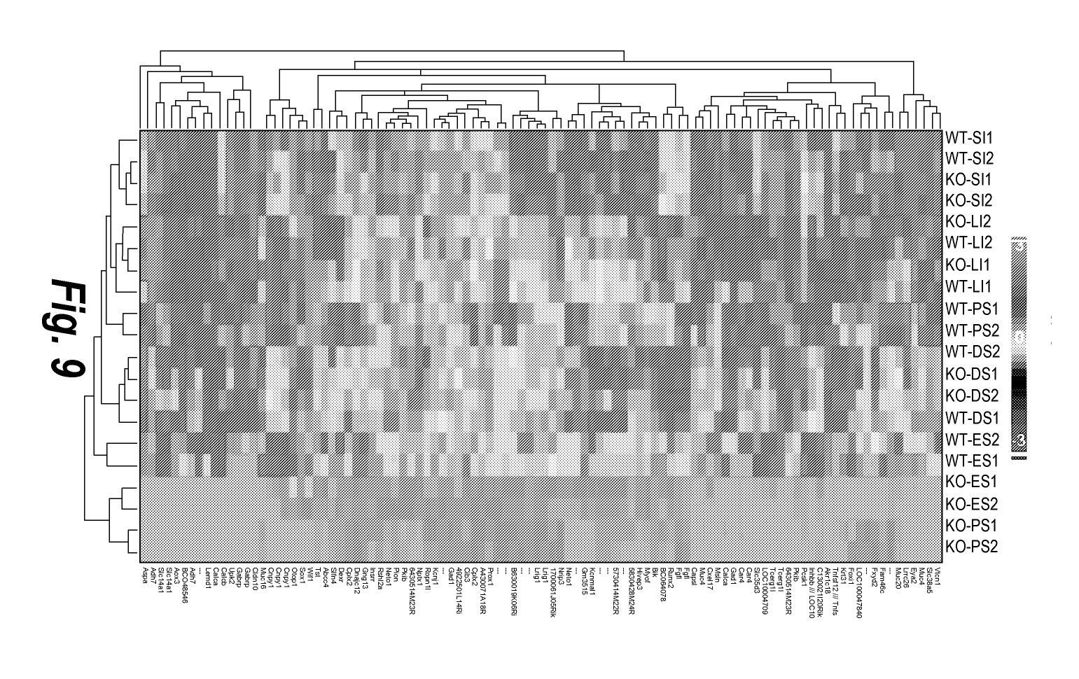

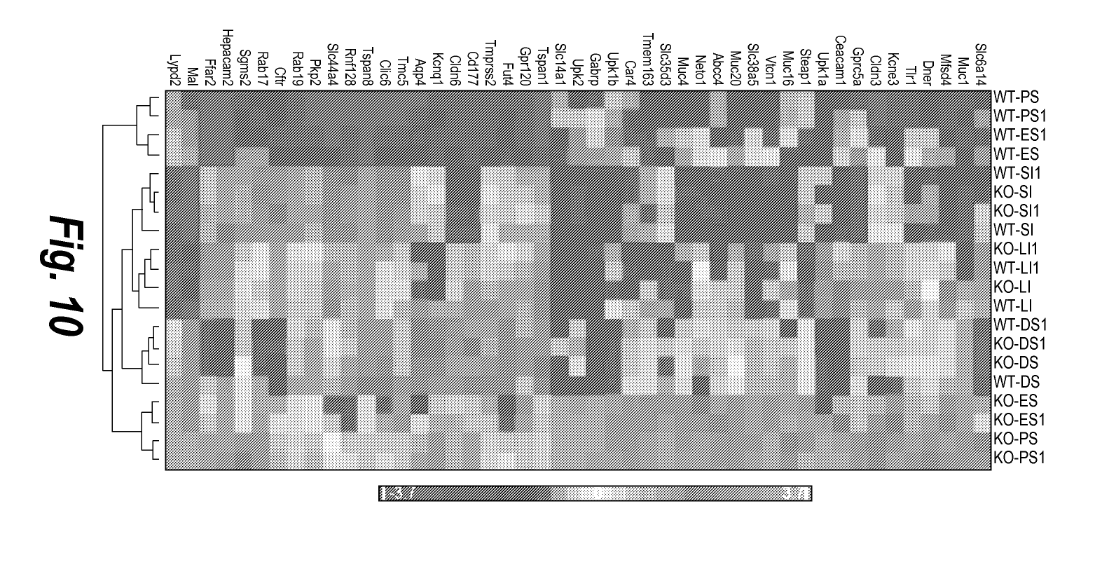

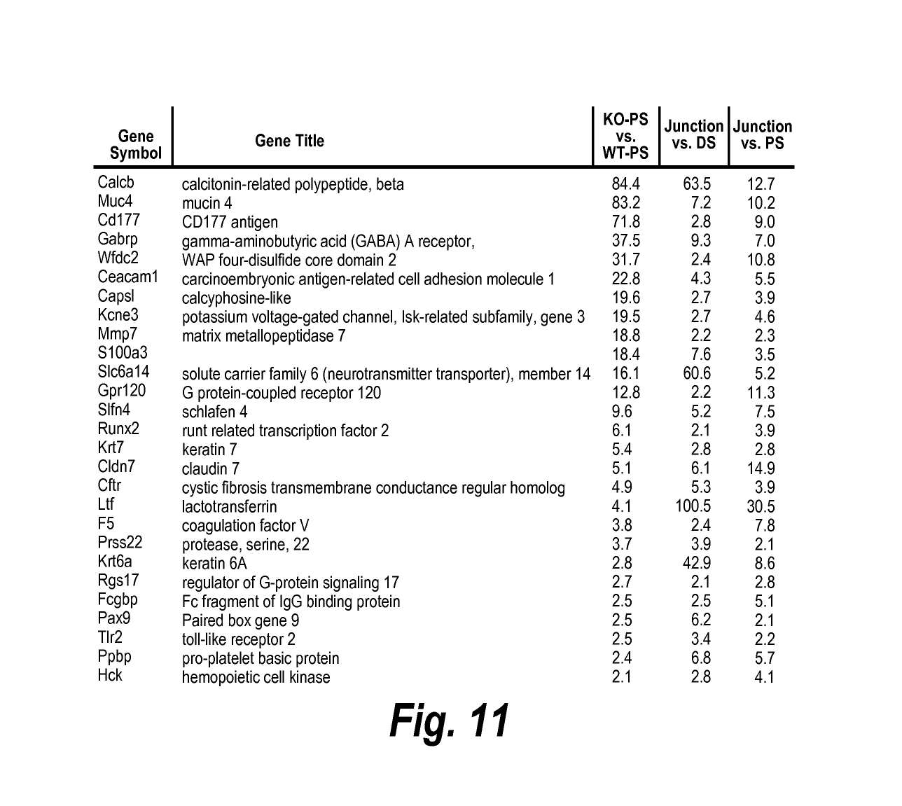

The present invention is based, in part, on the discovery that a unique population of primitive epithelial cells give rise to the metaplasia underlying esophageal and gastric adenocarcinoma. Transcriptome analysis of RNA derived by microdissection from this population of cells led to the remarkable discovery that these cells have a distinct molecular signature. In particular, a number of genes were identified as being upregulated in these cells. Moreover, a subset of these genes (set forth below in Tables 1-5, 15 and 16 and FIGS. 9-11, the sequences of which are each specifically incorporated herein by reference to their respective RefSeq Transcript ID numbers) were determined to be useful diagnostically for the identification of these primitive epithelial cells and/or as target molecules for therapeutics designed to kill or inhibit growth of these cells. Accordingly, the present invention makes use of the identified genes to provide methods and compositions for diagnosing, imaging, treating or preventing metaplasia (e.g., esophageal metaplasia). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

TABLE 1
Genes upregulated in Barrett's-like metaplasia in p63 null mice

| Gene Symbol | Gene Title | RefSeq Transcript ID |
|---|---|---|
| Foxi1 | forkhead box I1 | NM_023907 |
| 6430514M23Rik | RIKEN cDNA | — |
| | 6430514M23 gene | |
| Tcerg1\| | transcription | NM_183289 /// |
| | elongation regulator 1- | XM_916561 |
| | like | |
| Akr1c18 | aldo-keto reductase | NM_134066 |
| | family 1, member C18 | |
| Gad1 | glutamic acid | NM_008077 |
| | decarboxylase 1 | |
| BC048546 | cDNA sequence | NM_001001179 |
| | BC048546 | |
| Slc14a1 | solute carrier family | NM_028122 |
| | 14 (urea transporter), | |
| | member 1 | |
| LOC100047091 /// | hypothetical protein | NM_028135 /// |
| Tmem163 | LOC100047091 /// | XM_001477366 |
| | transmembrane | |
| | protein 163 | |
| Tcerg1\| | transcription | NM_183289 /// |
| | elongation regulator 1- | XM_916561 |
| | like | |
| Aox3 | aldehyde oxidase 3 | NM_023617 |
| Slc14a1 | solute carrier family | NM_028122 |
| | 14 (urea transporter), | |
| | member 1 | |
| Upk2 | uroplakin 2 | NM_009476 |
| Gm3515 | predicted gene 3515 | XM_001477025 |
| Pkib | protein kinase inhibitor | NM_001039050 /// |
| | beta, cAMP | NM_001039051 /// |
| | dependent, testis | NM_001039052 /// |
| | specific | NM_001039053 /// |
| | | NM_008863 |
| Inhbb /// | inhibin beta-B /// | NM_008381 /// |
| LOC100046802 | similar to Inhbb | XM_001476835 |
| | protein | |
| Cnpy1 | canopy 1 homolog | NM_175651 |
| | (zebrafish) | |
| Pkib | protein kinase inhibitor | NM_001039050 /// |
| | beta, cAMP | NM_001039051 /// |
| | dependent, testis | NM_001039052 /// |
| | specific | NM_001039053 /// |
| | | NM_008863 |
| 6430514M23Rik | RIKEN cDNA | — |
| | 6430514M23 gene | |
| Dnajc12 | DnaJ (Hsp40) | NM_013888 |
| | homolog, subfamily C, | |
| | member 12 | |
| Pcsk1 | proprotein convertase | NM_013628 |
| | subtilisin/kexin type 1 | |
| Calca | calcitonin/calcitonin- | NM_001033954 /// |
| | related polypeptide, | NM_007587 |
| | alpha | |
| Slc38a5 | solute carrier family | NM_172479 |
| | 38, member 5 | |
| Lemd1 | LEM domain | NM_001033250 |
| | containing 1 | |
| Wif1 | Wnt inhibitory factor 1 | NM_011915 |
| Vtcn1 | V-set domain | NM_178594 |
| | containing T cell | |
| | activation inhibitor 1 | |
| B630019K06Rik | RIKEN cDNA | NM_175327 |
| | B630019K06 gene | |
| Adh7 | alcohol | NM_009626 |
| | dehydrogenase 7 | |
| | (class IV), mu or | |
| | sigma polypeptide | |
| Sox1 | SRY-box containing | NM_009233 |
| | gene 1 | |
| Cnpy1 | canopy 1 homolog | NM_175651 |
| | (zebrafish) | |
| Nrip3 | nuclear receptor | NM_020610 |
| | interacting protein 3 | |
| Adh7 | alcohol | NM_009626 |
| | dehydrogenase 7 | |
| | (class IV), mu or | |
| | sigma polypeptide | |
| Slc35d3 | solute carrier family | NM_029529 |
| | 35, member D3 | |
| Cnpy1 | canopy 1 homolog | NM_175651 |
| | (zebrafish) | |
| Tnfsf12 /// Tnfsf12- | tumor necrosis factor | NM_001034097 /// |
| tnfsf13 /// Tnfsf13 | (ligand) superfamily, | NM_001034098 /// |
| | member 12 /// tumor | NM_001159503 /// |
| | necrosis factor | NM_001159505 /// |
| | | NM_011614 // |
| Eya2 | eyes absent 2 | NM_010165 |
| | homolog (Drosophila) | |
| Fxyd2 | FXYD domain- | NM_007503 /// |
| | containing ion | NM_052823 |
| | transport regulator 2 | |
| Bik | BCL2-interacting killer | NM_007546 |
| Krt31 | keratin 31 | NM_010659 |
| Calcb | calcitonin-related | NM_054084 |
| | polypeptide, beta | |
| Neto1 | neuropilin (NRP) and | NM_144946 |
| | tolloid (TLL)-like 1 | |
| Pion | pigeon homolog | NM_175437 |
| | (Drosophila) | |
| Myof | myoferlin | NM_001099634 /// |
| | | XM_001480162 /// |
| | | XM_001480167 /// |
| | | XM_283556 |
| Lrig1 | leucine-rich repeats | NM_008377 |
| | and immunoglobulin- | |
| | like domains 1 | |
| Fgf1 | fibroblast growth | NM_010197 |
| | factor 1 | |
| Hivep3 | human | NM_010657 |
| | immunodeficiency | |
| | virus type I enhancer | |
| | binding protein 3 | |
| Insrr | insulin receptor- | NM_011832 |
| | related receptor | |
| Neto1 | neuropilin (NRP) and | NM_144946 |
| | tolloid (TLL)-like 1 | |
| Cldn10 | claudin 10 | NM_001160096 /// |
| | | NM_001160097 /// |
| | | NM_001160098 /// |
| | | NM_001160099 /// |
| | | NM_021386 // |
| Gad1 | glutamic acid | NM_008077 |
| | decarboxylase 1 | |
| Cib3 | calcium and integrin | NM_001080812 /// |
| | binding family | XM_356089 /// |
| | member 3 | XM_904518 |
| Capsl | calcyphosine-like | NM_029341 |
| — | — | — |
| Nptx1 | neuronal pentraxin 1 | NM_008730 |
| Muc4 | mucin 4 | NM_080457 |
| Calca | calcitonin/calcitonin- | NM_001033954 /// |
| | related polypeptide, | NM_007587 |
| | alpha | |
| Lrig1 | leucine-rich repeats | NM_008377 |
| | and immunoglobulin- | |
| | like domains 1 | |
| Gabrp | gamma-aminobutyric | NM_146017 |
| | acid (GABA) A | |
| | receptor, pi | |
| Cxcl17 | chemokine (C—X—C | NM_153576 |
| | motif) ligand 17 | |
| Lrrc26 | leucine rich repeat | NM_146117 |
| | containing 26 | |
| LOC100047840 /// | similar to stem cell | NM_019992 /// |
| Stap1 | adaptor protein STAP- | XM_001479407 /// |
| | 1 /// signal transducing | XM_001479415 |
| | adaptor famil | |
| Msln | mesothelin | NM_018857 |
| 5730414M22Rik | RIKEN cDNA | — |
| | 5730414M22 gene | |
| Aspa | aspartoacylase | NM_023113 |
| Gng13 | guanine nucleotide | NM_022422 |
| | binding protein (G | |
| | protein), gamma 13 | |
| Muc4 | mucin 4 | NM_080457 |
| Car4 | carbonic anhydrase 4 | NM_007607 |
| A430071A18Rik | RIKEN cDNA | — |
| | A430071A18 gene | |
| C130021\|20Rik | Riken cDNA | NM_177842 |
| | C130021\|20 gene | |
| Cplx2 | complexin 2 | NM_009946 |
| Runx2 | runt related | NM_001145920 /// |
| | transcription factor 2 | NM_001146038 /// |
| | | NM_009820 |
| Dcxr | dicarbonyl L-xylulose | NM_026428 |
| | reductase | |
| 1700061J05Rik | RIKEN cDNA | NM_001163612 /// |
| | 1700061J05 gene | NM_001163613 /// |
| | | NM_028522 /// |
| | | XM_181371 /// |
| | | XM_911673 |
| Fam46c | family with sequence | NM_001142952 /// |
| | similarity 46, member C | XR_001536 /// |
| | | XR_002338 /// |
| | | XR_005163 |
| Muc16 | mucin 16 | XM_001476091 /// |
| | | XM_911929 |
| Cplx2 | complexin 2 | NM_009946 |
| 5830428M24Rik | RIKEN cDNA | — |
| | 5830428M24 gene | |
| Kcnj1 | potassium inwardly- | NM_019659 |
| | rectifying channel, | |
| | subfamily J, member 1 | |
| Gabrp | gamma-aminobutyric | NM_146017 |
| | acid (GABA) A | |
| | receptor, pi | |
| Car4 | carbonic anhydrase 4 | NM_007607 |
| Kcnma1 | Potassium large | NM_010610 |
| | conductance calcium- | |
| | activated channel, | |
| | subfamily M, alpha | |
| | member | |
| Otop1 | otopetrin 1 | NM_172709 |
| Prox1 | prospero-related | NM_008937 |
| | homeobox 1 | |
| Abcc4 | ATP-binding cassette, | NM_001033336 /// |
| | sub-family C | NM_001163675 /// |
| | (CFTR/MRP), | NM_001163676 |
| | member 4 | |
| BC064078 | cDNA sequence | NR_015455 /// |
| | BC064078 | XR_034925 /// |
| | | XR_035011 |
| Fgf1 | fibroblast growth | NM_010197 |
| | factor 1 | |
| Tst | thiosulfate | NM_009437 |
| | sulfurtransferase, | |
| | mitochondrial | |
| Rshl2a | radial spokehead-like | NM_025789 |
| | 2A | |
| Muc20 | mucin 20 | NM_001145874 /// |
| | | NM_146071 |
| 4922501L14Rik | RIKEN cDNA | NM_175176 /// |
| | 4922501L14 gene | XM_001481326 /// |
| | | XR_032207 |
| Ropn1\| | ropporin 1-like | NM_145852 |
| Slfn4 | schlafen 4 | NM_011410 |

TABLE 2
Cell surface marker genes upregulated in Barrett's like metaplasia.

| Gene Symbol | Gene Title |
|---|---|
| slc6a14 | |
| muc1 | mucin 1 |
| MFsd4 | |
| DNER | |
| Tlr1 | |
| Kcne3 | |
| Cldn3 | |
| Gprc5a | |
| Ceacam1 | |
| Upk1a | |
| Steap1 | |
| Muc16 | mucin 1 |
| Vtcn1 | |
| Slc38a5 | |
| Muc20 | |
| Abcc4 | |
| Neto1 | |
| Muc4 | mucin 4 |
| Slc35d3 | |
| Tmem163 | |
| Car4 | |
| Slc14a1 | |
| Hepacam2 | |
| cd177 | |
| kcnq1 | |
| sgms2 | |
| rab17 | |

TABLE 3
Genes upregulated in cells of the squamocolumnar junction of the
stomach.

| Gene Symbol | Gene Title |
|---|---|
| LOC632073 /// U46068 | similar to long palate, lung and nasal |
| | epithelium carcinoma associated 1 isoform |
| Ltf | lactotransferrin |
| Defb4 | defensin beta 4 |
| Ugt8a | UDP galactosyltransferase 8A |
| Mcpt2 | mast cell protease 2 |
| Onecut2 | one cut domain, family member 2 |
| Mcpt1 | mast cell protease 1 |
| Gcg | glucagon |
| Cldn7 | claudin 7 |
| Calcb | calcitonin-related polypeptide, beta |
| Pigr | polymeric immunoglobulin receptor |
| Gpr120 | G protein-coupled receptor 120 |
| Pate4 | prostate and testis expressed 4 |
| Wfdc2 | WAP four-disulfide core domain 2 |
| Rgs13 | regulator of G-protein signaling 13 |
| Muc4 | mucin 4 |
| Apob | apolipoprotein B |
| Gm14446 | predicted gene 14446 |
| U46068 | cDNA sequence U46068 |
| Cd177 | CD177 antigen |
| Itih2 | inter-alpha trypsin inhibitor, heavy chain 2 |
| Spib | Spi-B transcription factor (Spi-1/PU.1 related) |
| Krt6a | keratin 6A |
| F5 | coagulation factor V |
| Hamp | hepcidin antimicrobial peptide |
| Slfn4 | schlafen 4 |
| Trpm5 | transient receptor potential cation channel, |
| | subfamily M, member 5 |
| Spink12 | serine peptidase inhibitor, Kazal type 11 |
| Hsd11b2 | hydroxysteroid 11-beta dehydrogenase 2 |
| Gabrp | gamma-aminobutyric acid (GABA) A receptor, |
| | pi |
| Ceacam1 | carcinoembryonic antigen-related cell adhesion |
| | molecule 1 |
| Cldn2 | claudin 2 |
| BC100530 /// Stfa1 | cDNA sequence BC100530 /// stefin A1 |
| Siglec5 | sialic acid binding Ig-like lectin 5 |
| Reg3g | regenerating islet-derived 3 gamma |
| Gsdmc2 /// | gasdermin C2 /// hypothetical protein |
| LOC100045250 | LOC100045250 |
| 2010205A11Rik /// | RIKEN cDNA 2010205A11 gene /// predicted |
| Gm10883 /// Gm1420 /// | gene 10883 /// predicted gene 1420 /// |
| Gm7202 /// Igk /// | |
| Igk-C /// Igk-V28 /// | |
| LOC100047628 | |
| Ppbp | pro-platelet basic protein |
| Expi | extracellular proteinase inhibitor |
| 2310038E17Rik | RIKEN cDNA 2310038E17 gene |
| Slc6A14 | solute carrier family 6 (neurotransmitter |
| | transporter), member 14 |
| Fcgbp | Fc fragment of IgG binding protein |
| Aqp5 /// LOC100046616 | aquaporin 5 /// similar to aquaporin 5 |
| Naip5 | NLR family, apoptosis inhibitory protein 5 |
| Gm10883 /// Gm1420 /// | predicted gene 10883 /// predicted gene 1420 |
| Gm7202 /// Igk /// | /// predicted gene 7202 /// immunog |
| Igk-C /// Igk-V28 /// | |
| LOC100047628 | |
| Dclk1 | doublecortin-like kinase 1 |
| Stfa2l1 | stefin A2 like 1 |
| Kcne3 | potassium voltage-gated channel, Isk-related |
| | subfamily, gene 3 |
| Pcdh24 | protocadherin 24 |
| Igh /// Igh-2 /// | immunoglobulin heavy chain complex /// |
| Igh-VJ558 /// | immunoglobulin heavy chain 2 (serum IgA) |
| LOC544903 | |
| Stfa3 | stefin A3 |
| Trpm5 | transient receptor potential cation channel, |
| | subfamily M, member 5 |
| Igh /// Igh-2 /// | immunoglobulin heavy chain complex /// |
| Igh-VJ558 /// | immunoglobulin heavy chain 2 (serum IgA) |
| LOC544903 | |
| Igj | immunoglobulin joining chain |
| Gpa33 | glycoprotein A33 (transmembrane) |

Also provided is a subset of genes from the human isolated clonal population of Barrett's esophagus progenitor cells (set forth below in Table 4, the sequences of which are each specifically incorporated herein by reference to their respective RefSeq Transcript ID numbers). Each of these genes is expressed at, at least, 10% of the expression of actin in these cells. These genes were determined to be useful diagnostically for the identification of these cells and/or as target molecules for therapeutics designed to kill or inhibit growth of these cells. Accordingly, the present invention makes use of the identified genes to provide methods and compositions for diagnosing, imaging, treating or preventing metaplasia (e.g., esophageal metaplasia). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

TABLE 4

| Gene Symbol | Accession No. |
|---|---|
| GSTM4 | NM_000850 |
| SLC16A4 | NM_004696 |
| CMBL | NM_138809.3 |
| CEACAM6 | NM_002483 |
| NR5A2 | NM_205860 |
| CFTR | NM_000492 |
| GCNT3 | NM_004751 |

Also provided is a subset of genes from the human isolated clonal population of Barrett's esophagus progenitor cells (set forth below in Table 5, the sequences of which are each specifically incorporated herein by reference to their respective RefSeq Transcript ID numbers). These genes are upregulated in Barrett's esophagus progenitor cells when compared to their expression in squamous cell and gastric cardia progenitor cells. These genes were also determined to be useful diagnostically for the identification of these cells and/or as target molecules for therapeutics designed to kill or inhibit growth of these cells. Accordingly, the present invention makes use of the identified genes to provide methods and compositions for diagnosing, imaging, treating or preventing metaplasia (e.g., esophageal metaplasia). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

TABLE 5

| Gene Symbol | RefSeq |
|---|---|
| ODAM | NM_017855 |
| GSTM4 | NM_000850 |
| BICC1 | NM_001080512 |
| SLC16A4 | NM_004596 |
| NTS | NM_006183 |
| BAAT | NM_001701 |
| DDX43 | NM_018665 |
| MXRA5 | NM_015419 |
| FGF2 | NM_002006 |
| AK5 | NM_174858 |
| CCL28 | NM_148672 |
| HLA-DMB | NM_002118 |
| TNFRSF10C | NM_003841 |
| HS3ST5 | NM_153612 |
| CTH | NM_001902 |
| TGFB2 | NM_001135599 |
| CLDN10 | NM_182848 |
| SLC15A1 | NM_005073 |
| CYP2E1 | NM_000773 |
| GSTM2 | NM_000848 |
| LRRC6 | NM_012472 |
| CCBE1 | NM_133459 |
| STC2 | NM_003714 |
| NKX6-3 | NM_152568 |
| MATN2 | NM_002380 |
| USP44 | NM_032147 |

In certain embodiments, the isolated Barrett's esophagus progenitor cells described herein are negative for the expression of mRNA of any one or more of the genes shown in Table 6, the sequences of which are each specifically incorporated herein by reference to their respective RefSeq Transcript ID numbers.

TABLE 6
Negatively expressing genes

| Gene Symbol | RefSeq |
|---|---|
| SOX2 | NM_003106 |
| TP63 | NM_003722 |
| KRT20 | NM_019010 |
| GKN1 | NM_019617 |
| GKN2 | NM_182536 |
| FABP1 | NM_001443 |
| FABP2 | NM_000134 |
| Krt14 | NM_000526 |
| CXCL17 | NM_198477 |

In certain specific embodiments, the isolated Barrett's esophagus progenitor cells described herein are negative for the expression of Krt20, Sox2 and p63 mRNA. In other specific embodiments, the isolated Barrett's esophagus progenitor cells described herein are negative for the expression of SOX2, p63, KRT20, GKN1, GKN2, FABP1, FABP2, KRT14 and CXCL17.

In certain embodiments, the isolated Barrett's esophagus progenitor cells described herein are positive for the expression of any one or more mRNA of any one or more of the genes shown in Table 7, the sequences of which are each specifically incorporated herein by reference to their respective RefSeq Transcript ID numbers.

TABLE 7
Positively expressing genes

| Gene Symbol | RefSeq |
|---|---|
| GSTM4 | NM_000850 |
| SLC16A4 | NM_004696 |
| CMBL | NM_138809 |
| CEACAM6 | NM_002483 |
| NR5pA2 | NM_205860 |
| CFTR | NM_000492 |
| GCNT3 | NM_004751 |
| BICC1 | NM_001080512 |
| NTS | NM_006183 |

In certain specific embodiments, the isolated Barrett's esophagus progenitor cells described herein are positive for the expression of CEACAM6 mRNA. In other specific embodiments, the isolated Barrett's esophagus progenitor cells described herein are negative for the expression of CEACAM6, GSTM4, SLC16A4, CMBL, NR5A2, CFTR, GCNT3, BICC1 and NTS mRNA.

In other embodiments, the isolated Barrett's esophagus progenitor cells described herein are negative for the expression of any one or more of Sox2, p63, Krt20, GKN1/2, FABP1/2, KRT14 or CXCL17 mRNA and positive for the expression of any one or more of CEACAM6, GSTM4, SLC16A4, CMBL, NR5A2, CFTR, GCNT3, BICC1 or NTS mRNA. In certain specific embodiments, the isolated Barrett's esophagus progenitor cells described herein are positive for the expression of CEACAM6 mRNA and negative for the expression of Krt20, Sox2 and p63. In other specific embodiments, the isolated Barrett's esophagus progenitor cells described herein are negative for the expression of Sox2, p63, Krt20, GKN1/2, FABP1/2, KRT14 and CXCL17 mRNA and positive for the expression of CEACAM6, GSTM4, SLC16A4, CMBL, NR5A2, CFTR, GCNT3, BICC1 and NTS mRNA.

In certain embodiments, the human isolated clonal population of Barrett's esophagus progenitor cells disclosed herein are cultured with 5 mg/ml insulin, 10 ng/ml EGF, 2×10⁻⁹ M 3,3′,5-triiodo-L-thyronine, 0.4 mg/ml hydrocortisone, 24 mg/ml adenine, 1×10⁻¹⁰ M cholera toxin, 10 Jagged 1, 100 ng/ml Noggin, 125 ng/ml R Spondin 1, 2.5 μM Rock inhibitor in DMEM/Ham's F12 3:1 medium with 10% fetal bovine serum when the mRNA expression analysis is performed.

Also provided is a subset of genes from a human isolated clonal population of squamous progenitor cells (set forth below in Table 8, the sequences of which are each specifically incorporated herein by reference to their respective RefSeq Transcript ID numbers). Each of these genes is expressed at, at least, 10% of the expression of actin in these cells. These genes were determined to be useful diagnostically for the identification of these cells and/or to distinguish these cells from Barrett's esophagus progenitor cells, so that the Barrett's esophagus progenitor cells can be selectively ablated without damaging squamous progenitor cells. Accordingly, the present invention makes use of the identified genes to provide methods and compositions for diagnosing, imaging, treating or preventing metaplasia (e.g., esophageal metaplasia). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

TABLE 8

| Gene Symbol | Accession No. |
|---|---|
| S100A8 | NM_002964 |
| Krt14 | NM_000526 |
| SPRR1A | NM_005987 |
| CSTA | NM_005213 |

Also provided is a subset of genes from the human isolated clonal population of squamous progenitor cells (set forth below in Table 9, the sequences of which are each specifically incorporated herein by reference to their respective RefSeq Transcript ID numbers). These genes are upregulated in squamous progenitor cells when compared to their expression in Barrett's esophagus and gastric cardia progenitor cells. These genes were determined to be useful diagnostically for the identification of these cells and/or differentiation of these cells from Barrett's esophagus progenitor cells, so that the Barrett's esophagus progenitor cells can be selectively ablated without damaging squamous progenitor cells. Accordingly, the present invention makes use of the identified genes to provide methods and compositions for diagnosing, imaging, treating or preventing metaplasia (e.g., esophageal metaplasia). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

TABLE 9

| Gene Symbol | RefSeq |
|---|---|
| S100A8 | NM_002964 |
| DSG1 | NM_001942 |
| SPINK6 | NM_205841 |
| SPRR1B | NM_003125 |
| SERPINB13 | NM_012397 |
| DSC3 | NM_024423 |
| KRT14 | NM_000526 |
| KRT17 | NM_000422 |
| SPRR2D | NM_006945 |
| DSG3 | NM_001944 |
| A2ML1 | NM_144670 |
| TMEN45A | NM_018004 |
| SBSN | NM_198538 |
| KRT5 | NM_000424 |
| SPRR1A | NM_005987 |
| SERPINB7 | NM_003784 |
| TFPI2 | NM_006528 |
| IVL | NM_005547 |
| CAPNS2 | NM_032330 |
| DSC1 | NM_004948 |
| TP63 | NM_003722 |

In certain embodiments, the isolated squamous progenitor cells described herein are negative for the expression of any one or more of mRNA of any one or more of the genes shown in Table 10, the sequences of which are each specifically incorporated herein by reference to their respective RefSeq Transcript ID numbers.

TABLE 10
Negatively expressing genes

| Gene Symbol | RefSeq |
|---|---|
| SOX2 | NM_003106 |
| Krt20 | NM_019010 |
| CXCL17 | NM_198477 |
| CEACAM6 | NM_002483 |
| NR5A2 | NM_205860 |

In certain specific embodiments, the isolated squamous progenitor cells described herein are negative for the expression of CEACAM6 mRNA. In other specific embodiments, the isolated squamous progenitor cells described herein are negative for the expression of Sox2, Krt20, CXCL17 and CEACAM6 mRNA.

In certain embodiments, the isolated squamous progenitor cells described herein are positive for the expression of any one or more mRNA of any one or more of the genes shown in Table 11, the sequences of which are each specifically incorporated herein by reference to their respective RefSeq Transcript ID numbers.

TABLE 11
Positively expressing genes

| Gene Symbol | RefSeq |
|---|---|
| S100A8 | NM_002964 |
| Krt14 | NM_000526 |
| SPRR1A | NM_005987 |
| CSTA | NM_005213 |
| TP63 | NM_003722 |

In certain specific embodiments, the isolated squamous progenitor cells described herein are positive for the expression of p63 mRNA. In other specific embodiments, the isolated squamous progenitor cells described herein are negative for the expression of S100A8, Krt14, SPRR1A, CSTA and p63 mRNA.

In other embodiments, the isolated squamous progenitor cells described herein are negative for the expression of any one or more of Sox2, Krt20, GKN1/2, FABP1/2, CXCL17 or CEACAM6 mRNA and positive for the expression of any one or more of S100A8, Krt14, SPRR1A, CSTA or p63 mRNA. In certain specific embodiments, the isolated squamous progenitor cells described herein are positive for the expression of p63 mRNA and negative for the expression of CEACAM6. In other specific embodiments, the isolated squamous progenitor cells described herein are negative for the expression of Sox2, Krt20, GKN1/2, FABP1/2, CXCL17 and CEACAM6 mRNA and positive for the expression of S100A8, Krt14, SPRR1A, CSTA and p63 mRNA.

Also provided is a subset of genes from a human isolated clonal population of gastric cardia progenitor cells (set forth below in Table 12, the sequences of which are each specifically incorporated herein by reference to their respective RefSeq Transcript ID numbers). Each of these genes is expressed at, at least, 10% of the expression of actin in these cells. These genes were determined to be useful diagnostically for the identification of these cells and/or to distinguish these cells from Barrett's esophagus progenitor cells, so that the Barrett's esophagus progenitor cells can be selectively ablated without damaging gastric cardia progenitor cells. Accordingly, the present invention makes use of the identified genes to provide methods and compositions for diagnosing, imaging, treating or preventing metaplasia (e.g., esophageal metaplasia). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

TABLE 12

| Gene Symbol | Accession No. |
|---|---|
| CXCL17 | NM_198477 |
| CAPN6 | NM_014289 |
| PSCA | NM_005672 |
| GKN1 | NM_019617 |
| GKN2 | NM_182536 |
| MT1G | NM_005950 |
| SPINK4 | NM_014471 |

Also provided is a subset of genes from the human isolated clonal population of gastric cardia progenitor cells (set forth below in Table 13, the sequences of which are each specifically incorporated herein by reference to their respective RefSeq Transcript ID numbers). These genes are upregulated in gastric cardia progenitor cells when compared to their expression in Barrett's esophagus and squamous progenitor cells. These genes were determined to be useful diagnostically for the identification of these cells and/or to distinguish these cells from Barrett's esophagus progenitor cells, so that the Barrett's esophagus progenitor cells can be selectively ablated without damaging squamous progenitor cells. Accordingly, the present invention makes use of the identified genes to provide methods and compositions for diagnosing, imaging, treating or preventing metaplasia (e.g., esophageal metaplasia). However, it should be appreciated that such methods and compositions are not limited to diagnosing, imaging, treating or preventing metaplasia, but can be can be used more generally for diagnosing, imaging, treating or preventing any disease arising from or containing cells that share the molecular signature disclosed herein. Such diseases include, without limitation, dysplasia (e.g., esophageal and gastric dysplasia), adenocarcinoma (e.g., esophageal, gastric and pancreatic adenocarcinoma), pancreatic intraepithelial neoplasia, inflammatory bowel disease (e.g., Crohn's disease and ulcerative colitis), and micropapillary carcinoma.

TABLE 13

| Gene Symbol | RefSeq |
|---|---|
| CXCL17 | NM_198477 |
| LOC84740 | NR_026892 |
| KIAA1324 | NM_020775 |
| MT1M | NM_176870 |
| C20orf114 | NM_033197 |
| MT1A | NM_005946 |
| ORM2 | NM_000608 |
| CAPN6 | NM_014289 |
| CAPN9 | NM_006615 |
| PSCA | NM_005672 |
| SLC26A9 | NM_052934 |
| SOX2OT | NR_004053 |
| GABRP | NM_014211 |
| UGT2B15 | NM_001076 |
| ITGBL1 | NM_004791 |
| UGT1A9 | NM_021027 |
| PIK3C2G | NM_004570 |
| GKN1 | NM_019617 |
| SCGB2A1 | NM_002407 |
| PTER | NM_030664 |
| GPR64 | NM_001079858 |
| LUM | NM_002345 |
| HRASLS2 | NM_017878 |
| GKN2 | NM_182536 |
| MRAP2 | NM_138409 |
| MAL | NM_002371 |
| SIM2 | NM_009586 |
| ORM1 | NM_000607 |
| FBP2 | NM_003837 |
| ALDH3A1 | NM_000691 |
| C11orf92 | NM_207429 |
| NPSR1 | NM_207172 |
| ARL14 | NM_025047 |
| CAPN13 | NM_144575 |
| RAB37 | NM_175738 |
| CYP4F12 | NM_023944 |
| PCDHB2 | NM_018936 |
| MGAM | NM_004668 |
| TCEA3 | NM_003196 |

In certain embodiments, the isolated gastric cardia progenitor cells described herein are negative for the expression of any one or more mRNA of any one or more of the genes shown in Table 14, the sequences of which are each specifically incorporated herein by reference to their respective RefSeq Transcript ID numbers.

TABLE 14
Negatively expressing genes

| Gene Symbol | RefSeq |
|---|---|
| CEACAM6 | NM_002483 |
| TP63 | NM_003722 |
| FABP1 | NM_001443 |
| FABP2 | NM_000134 |
| Krt14 | NM_000526 |
| Krt20 | NM_019010 |

In certain specific embodiments, the isolated gastric cardia progenitor cells described herein are negative for the expression of CEACAM6 mRNA. In other specific embodiments, the isolated gastric cardia progenitor cells described herein are negative for the expression of CEACAM6, p63, FABP1/2, Krt14 and Krt20 mRNA.

In certain embodiments, the isolated gastric cardia progenitor cells described herein are positive for the expression of any one or more mRNA of any one or more of the genes shown in Table 15, the sequences of which are each specifically incorporated herein by reference to their respective RefSeq Transcript ID numbers.

TABLE 14
Positively expressing genes

| Gene Symbol | RefSeq |
|---|---|
| CXCL17 | NM_198477 |
| CAPN6 | NM_014289 |
| CAPN9 | NM_006615 |
| PSCA | NM_005672 |
| SOX2 | NM_003106 |
| GKN1 | NM_019617 |
| GKN2 | NM_182536 |
| MT1G | NM_005950 |
| SPINK4 | NM_014471 |

In other specific embodiments, the isolated gastric cardia progenitor cells described herein are negative for the expression of CXCL17, CAPN6, CAPN9, PSCA, GKN1, GKN2, MT1G, SPINK4 and SOX2 mRNA.

In other embodiments, the isolated gastric cardia progenitor cells described herein are negative for the expression of any one or more of CEACAM6, p63, FABP1/2, Krt14 or Krt20 mRNA and positive for the expression of any one or more of CXCL17, CAPN6, CAPN9, PSCA, GKN1, GKN2, MT1G, SPINK4 or SOX2 mRNA. In other specific embodiments, the isolated gastric cardia progenitor cells described herein are negative for the expression of CEACAM6, p63, FABP1/2, Krt14 and Krt20 mRNA and positive for the expression of CXCL17, CAPN6, CAPN9, PSCA, GKN1, GKN2, MT1G, SPINK4 and SOX2 mRNA.

### B. Methods of Treatment

In one aspect, the invention provides methods for treating or preventing metaplasia (e.g., esophageal metaplasia). The methods of the invention generally comprise administering to a subject a therapeutic amount of an agent that decreases the expression and/or biological activity of one or more of the genes set forth in Tables 1-5 and FIGS. 9-11.

Any agent that causes a decrease in the expression and/or biological activity of the desired gene(s) is suitable for use in the methods of the invention. Suitable agents include, without limitation, antibodies, antibody-like molecules, aptamers, peptides, antisense oligonucleotides, small molecules or RNAi agents. In some embodiments, the agent decreases the amount of mRNA of the target gene. In other embodiments the agent decreases the expression of the protein product of the targeted gene. In other embodiments, the agent inhibits the biological activity of the protein product of the targeted gene (e.g., enzymatic activity or transcriptional activity). Such agents can be identified, for example, using the screening assays described herein.

In another aspect, the invention provides methods for treating or preventing metaplasia (e.g., esophageal metaplasia). The methods of the invention generally comprise administering a therapeutic amount of an agent that specifically binds to a cell surface polypeptide encoded by one of the genes set forth in Tables 1-5, 15 and 16 and FIGS. 9-11, wherein said agent is linked to one or more cytotoxic moiety.

Any agent that binds to the desired cell surface polypeptide is suitable for use in the methods of the invention. Suitable agents include, without limitation, antibodies, antibody-like molecules, aptamers, peptides, cell surface receptor ligand, or small molecules. In a preferred embodiment, the agent is an antibody, antibody-like molecule or cell surface receptor ligand.

In certain embodiments, cell surface polypeptides are targeted that are highly expressed in the Barrett's Esophagus progenitor cell but not in squamous cell progenitor cells that may be located nearby. The squamous cell progenitor cell described above and its mRNA expression profile compared to the profile of the clonal population of Barrett's Esophagus progenitor cells. Table 15 shows the mRNA from gene that were most highly expressed in clonal population of Barrett's Esophagus progenitor cells compared to the isolated squamous cell progenitor cell the sequences of which are each specifically incorporated herein by reference to their respective RefSeq Transcript ID numbers. Shaded genes in Table 15 are cell surface proteins.

TABLE 15

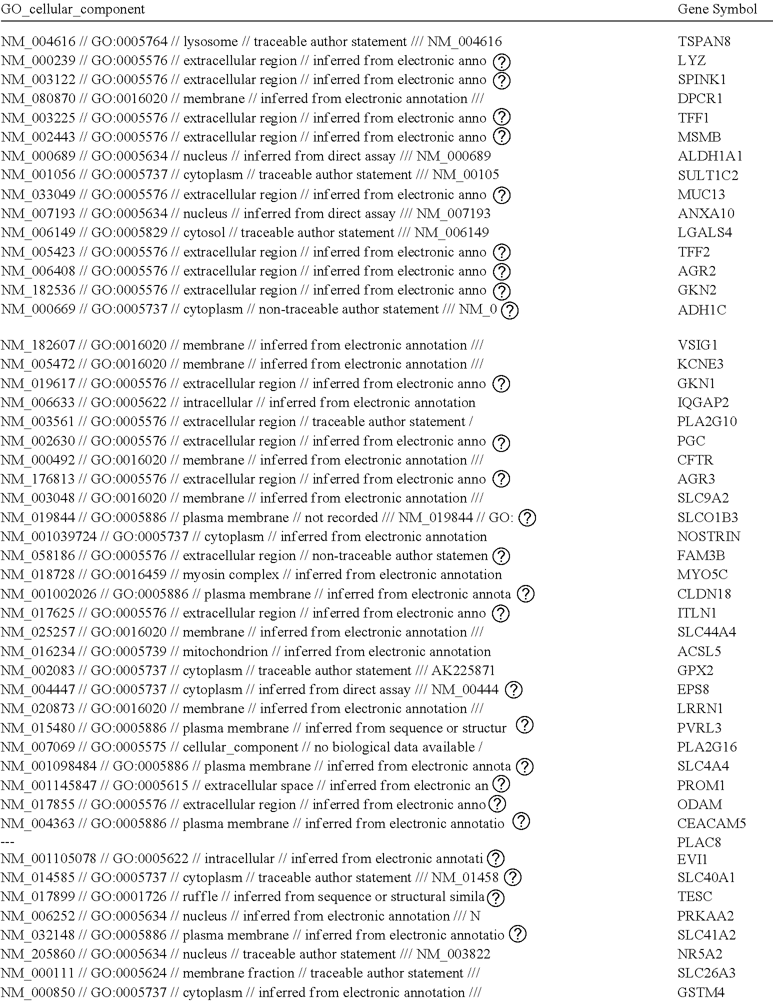

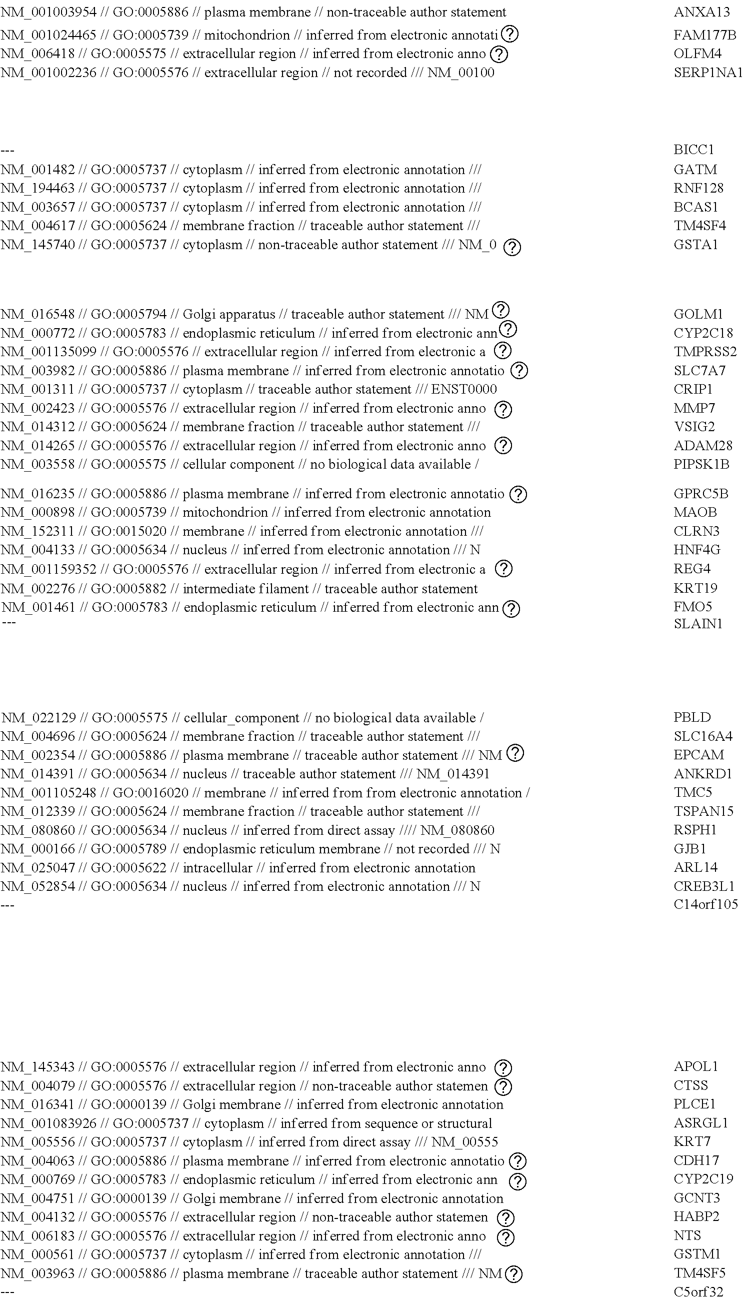

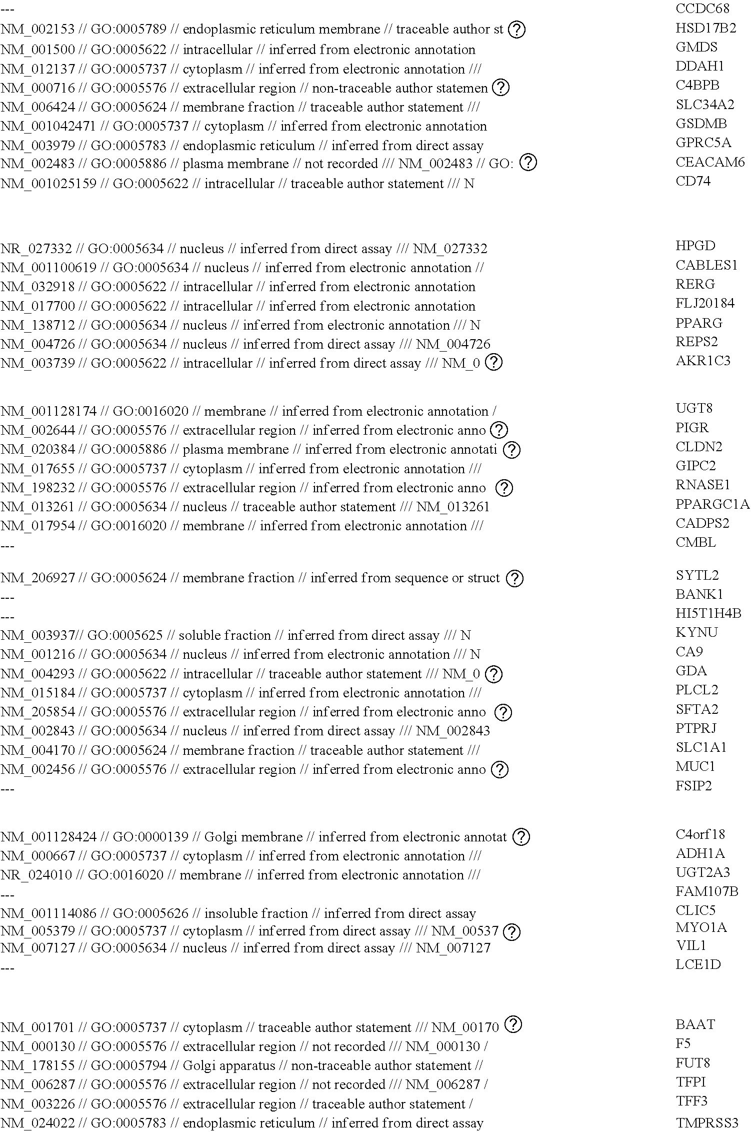

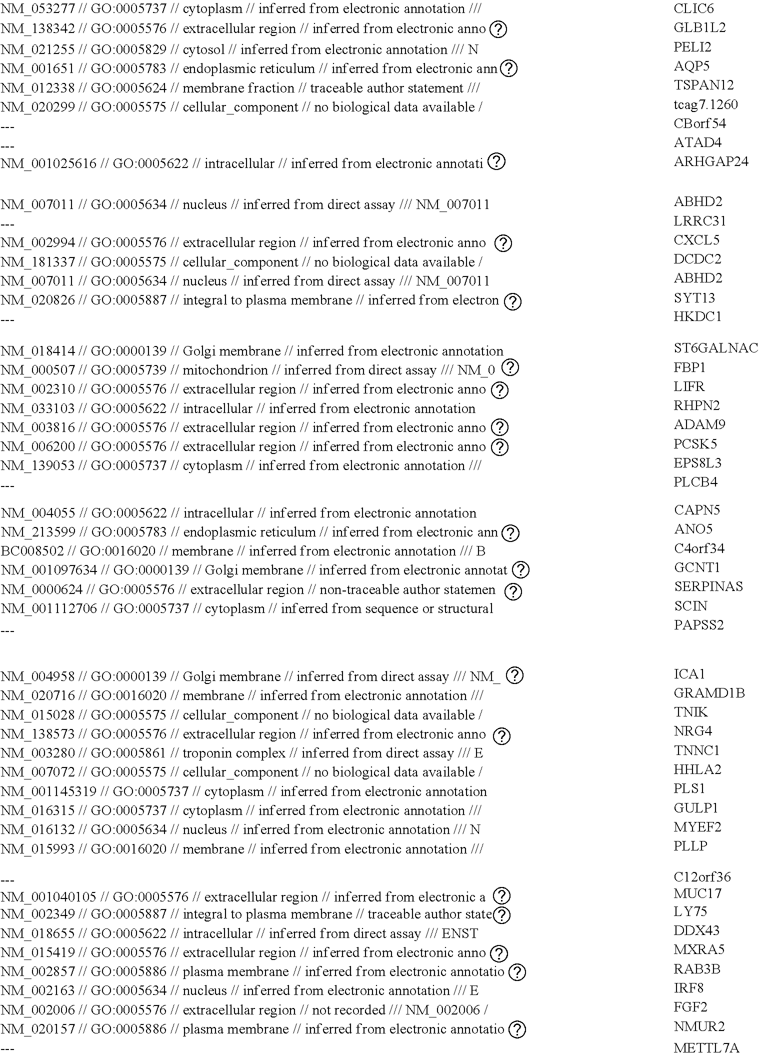

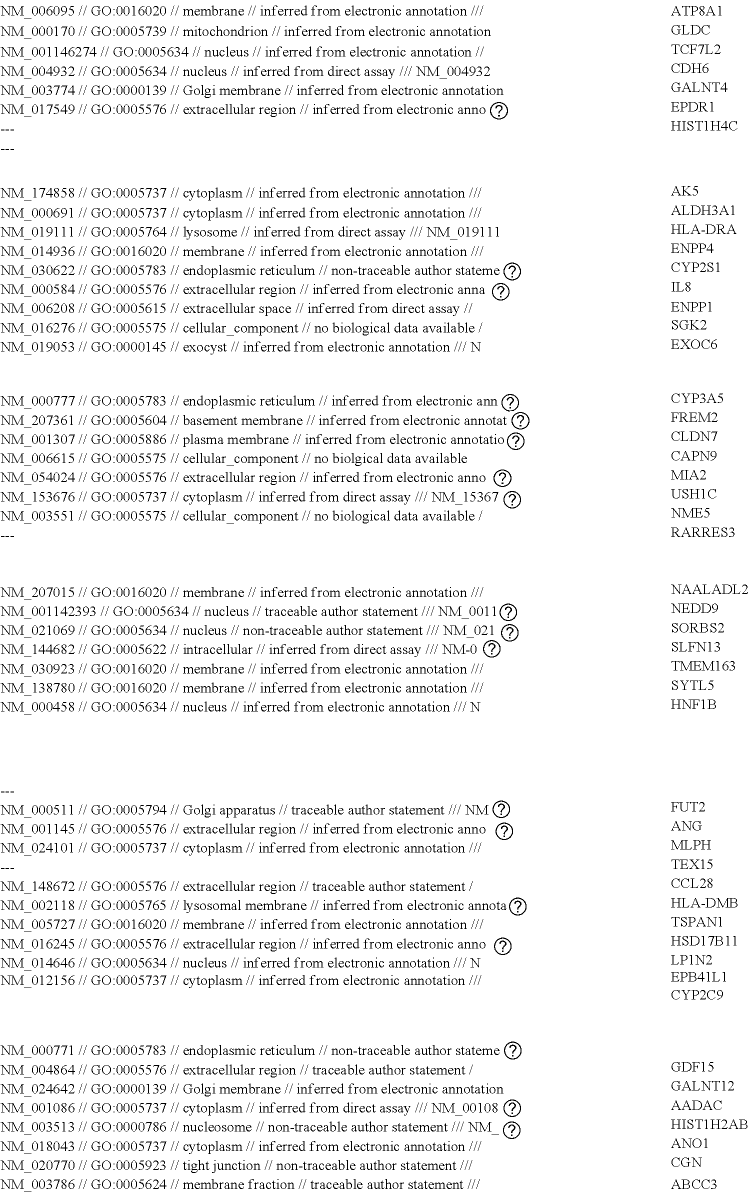

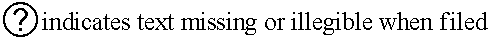

In certain embodiments, cell surface polypeptides are targeted that are highly expressed in the Barrett's Esophagus progenitor cell but not in gastric cardia cell progenitor cells that may be located nearby. The gastric cardia cell progenitor cell described above and its mRNA expression profile compared to the profile of the clonal population of Barrett's Esophagus progenitor cells. Table 16 shows the mRNA from gene that were most highly expressed in clonal population of Barrett's Esophagus progenitor cells compared to the isolated squamous cell progenitor cell the sequences of which are each specifically incorporated herein by reference to their respective RefSeq Transcript ID numbers. Shaded genes in Table 16 are cell surface proteins.

TABLE 16

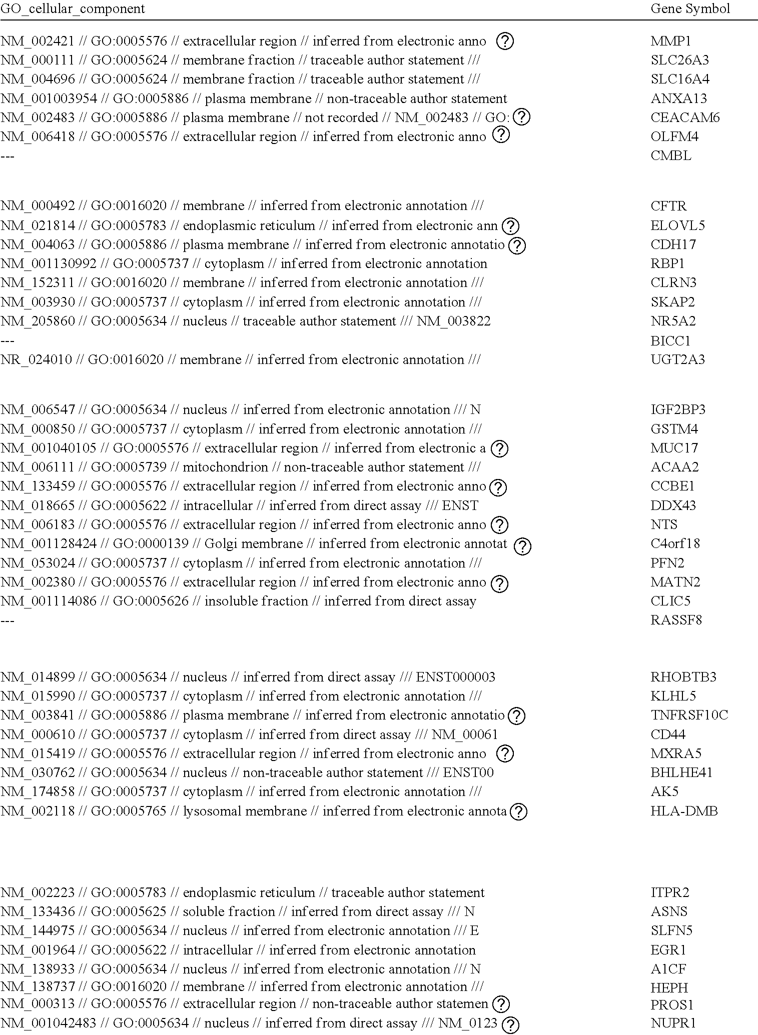

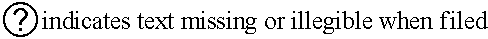

Any cytotoxic moiety is suitable for use in the methods of the invention, including, without limitation, radioactive isotopes, chemotoxins, or toxin proteins. Suitable radioactive isotopes include, without limitation, iodine¹³¹, indium¹¹¹, yttrium⁹⁰, and lutetium¹⁷⁷. Suitable chemotoxins include, without limitation, anthracyclines (e.g., daunorubicin (formerly daunomycin) and doxorubicin), antibiotics, taxol, cytochalasin B, gramicidin D, ethidium bromide, emetine, mitomycin, etoposide, tenoposide, colchicin, doxorubicin, daunorubicin, dihydroxy anthracin dione, mitoxantrone, mithramycin, actinomycin D, I-dehydrotestosterone, glucocorticoids, procaine, tetracaine, lidocaine, propranolol, antimetabolites (e.g., 30 methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), (e.g., dactinomycin (formerly actinomycin), bleomycin, mithramycin, and anthramycin (AMC)), anti-mitotic agents (e.g., vincristine and vinblastine), duocarmycins, calicheamicins, maytansines and auristatins, and derivatives thereof. Suitable toxin proteins include, without limitation, bacterial toxins (e.g., diphtheria toxin, and plant toxins (e.g., ricin).

Additional cytotoxic moieties include a medoximil moiety, PPARγ inhibitors and NR5A2 activity modulator.

CMBL (carboxymethylenebutenolidase homolog; NP_{—}620164.1) is highly expressed in Barrett's esophagus progenitor. CMBL is a cysteine hydrolase of the dienelactone hydrolase family that is highly expressed in liver and small intestine. CMBL preferentially cleaves cyclic esters, and it activates medoxomil-ester prodrugs in which the medoxomil moiety is linked to an oxygen atom (Ishizuka et al., 2010, J. Biol. Chem. 285, 11892-11902, incorporated by reference, herein, in its entirety). Thus, in certain embodiments, cytotoxic moieties include prodrug versions of common cytotoxic molecules, such as medoxomil-linked chemotherapeutics, to selectively damage Barrett's esophagus progenitor cells without significantly affecting other cell types of the esophagus or stomach. Alternatively this strategy could be used to introduce any appropriate pro-drug based on medoxomil chemistry to selectively affect the stem cells of IM.

PPARgamma (NM 138712) and PPARgC1A (NM 013261) are highly overexpressed in Barrett's esophagus progenitor cells versus squamous stem cells that give rise to the esophagus. Therefore, in certain embodiments, the cytotoxic moiety is a modulator of PPARgamma. An example of an irreversible inhibitor of PPARgamma is GW-9662 (2-Chloro-5-nitro-N-phenyl-benzamide), which suppresses PPARgamma with a nanomolar IC50. Modulators of PPARgamma, such as the drug class of thiazolidinediones (TZDs) are used clinically for the treatment of insulin resistance Yki-Järvinen, N Engl J Med. 351, 1106-1118 (2004); Staels and Fruchart Diabetes 54, 2460-2470 (2004).

The liver receptor homolog-1 (LRH-1) also known as NR5A2 (nuclear receptor subfamily 5, group A, member 2; NM 205860) is a protein that in humans is encoded by the NR5A2 gene, plays a critical role in the regulation of development, cholesterol transport, bile acid homeostasis and steroidogenesis. Bernier et al. (1993). Mol. Cell. Biol. 13 (3): 1619; and Galarneau et al. (1998) Cytogenet. Cell Genet. 82 (3-4): 269. NR5A2 is one of 49 “nuclear receptors” in the human genome that together represent ligand-regulated transcription factors. About half of these nuclear receptors have known ligands (estrogen, androgens, thyroid hormone, retinoids, vitamin D, etc.), the other half are orphan receptors.

The inventors have discovered, such as based on gene expression analysis of the cloned stem cells from Barrett's esophagus and gastric intestinal metaplasia, that the expression of NR5A2 is 10-20-fold higher when compared to indigenous stem cells of the esophagus and stomach. Our analysis further suggests that NR5A2 is likely a key stem cell factor required for self-renewal of both of both Barrett's and gastric intestinal metaplasia, and is different from the key self-renewal factors in the esophagus and stomach. Therefore targeting NR5A2 with agents that specifically affect the level of expression and/or functioning of NR5A2 in BE and IM stem cells versus the esophagus or stomach stem cells may be a useful way to inhibit the growth of those target stem cells, and perhaps a means to selectively ablate the BE and/or IM stem cell populations. The modulatory agents can include, for example, nucleic acid therapeutics such as siRNA, antisense, decoys and the like, as well as intracellular antibodies and antibody mimetics, and small molecules.

While NR5A2 is an orphan nuclear receptor, but considerable efforts are underway to drug these orphan receptors using molecular docking into homologous ligand pockets within the NR5A2 structures. In certain embodiments, the NR5A2 modulator is an agonist, such as dilauroyl phosphatidylcholine, or an agonist having the structure

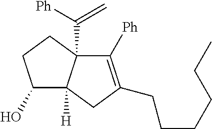 [SMILES: C=C(c1ccccc1)[C@@]12CC[C@@H](O)[C@@H]1CC(CCCCCC)=C2c1ccccc1]

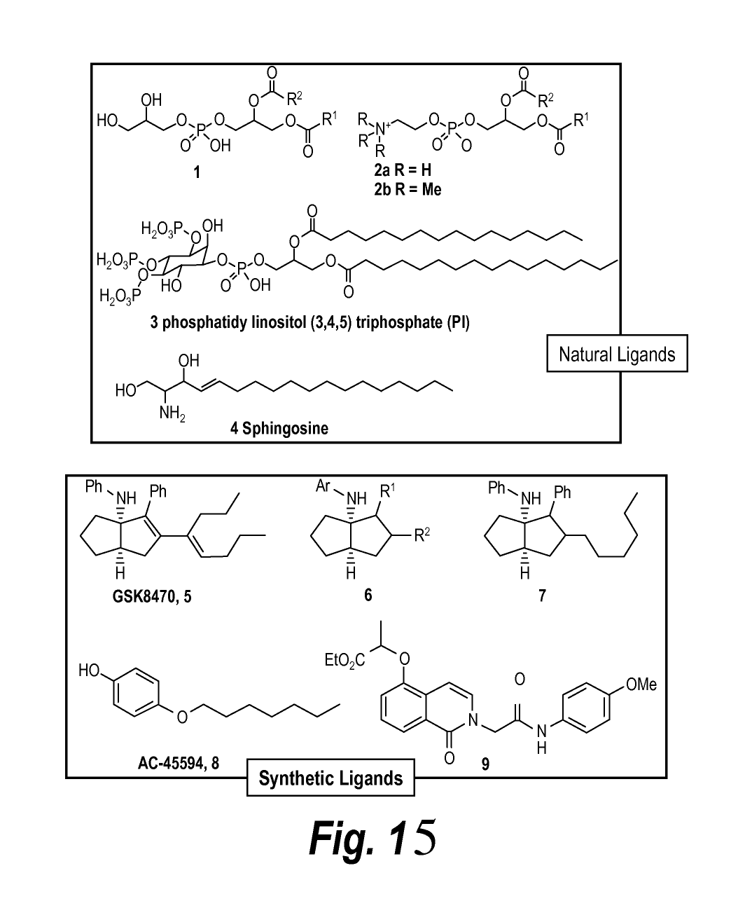

Other natural and synthetic modulators are disclosed in Whitby et al., (2011) J. Mol. Med. 54, 2266, and representative embodiments are shown in FIG. 15. Additional compounds can by synthesized from these parent compounds using standard medicinal chemistry.

In certain embodiments the cytotoxic moiety is linked directly (either covalently or non-covalently) to the agent. In other embodiments the cytotoxic moiety is incorporated into a biocompatible delivery vehicle that is in turn linked directly (either covalently or non-covalently) to the agent. Biocompatible delivery vehicles are well known in the art and include, without limitation, microcapsules, microparticles, nanoparticles, liposomes and the like.

Applicants have discovered that it is a primitive cell population residing at the squamocolumnar junction that is responsible for esophageal metaplasia. Accordingly, ablation of this cell population in normal, healthy individuals would protect those individuals from esophageal metaplasia and, in turn, from esophageal adenocarinoma. Thus, the present invention provides for both prophylactic and therapeutic methods of treatment. In some embodiments, the patient to be treated has been diagnosed as having metaplasia. In other embodiments, the patient to be treated does not have metaplasia.

According to the methods of the invention, the agent can be administered via any means appropriate to effect treatment. In some embodiments, the agent is administered parenterally. In other embodiments, the agent is administered orally. In a preferred embodiment, the agent is administered endoscopically to the esophageal squamocolumnar junction or to a site of esophageal metaplasia. Any endoscopic device or procedure capable of delivering an agent is suitable for use in the methods of the invention.

An agent of the invention typically is administered to the subject in a pharmaceutical composition. The pharmaceutical composition typically includes the agent formulated together with a pharmaceutically acceptable carrier. Pharmaceutical compositions can be administered in combination therapy, i.e., combined with other agents. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for oral, and parenteral administration (e.g., by injection or infusion).

In some embodiments, the expression of genes required for activation, division or growth of the stem cell can reduced or otherwise inhibited using a nucleic acid therapeutic. In preferred embodiments, the nucleic acid therapeutic is selectively cytotoxic or cytotoxic to the stem cell relative to other normal tissue in the alimentary canal, particularly adjacent tissues. In the case of the BE stem cell, preferable nucleic acid therapeutics are selectively cytotoxic or cytotoxic to the BE cell as relative to normal esophageal squamous epithelium and/or esophageal squamous stem cells and/or stomach cardia stem cells.

Exemplary nucleic acid therapeutics include, but are not limited to, antisense oligonucleotides, decoys, siRNAs, miRNAs, shRNAs and ribozymes. These agents can be delivered through a variety of routes of administration, but a preferred route is through local delivery, such as by local injection or endoscopic delivery. Moreover, the nucleic acid therapeutic can be modified with one or more moieties which promote uptake of the polynucleotide by the targeted stem cell. For instance, the modification can be a peptide or a peptidomimetic that enhances cell permeation, or a lipophilic moiety which enhances entrance into a cell. Exemplary lipophilic moieties include those chosen from the group consisting of a lipid, cholesterol, oleyl, retinyl, cholesteryl residues, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

RNA Interference Nucleic Acids

In particular embodiments, nucleic acid therapeutic is an RNA interference (RNAi) molecule. RNA interference methods using RNAi molecules may be used to disrupt the expression of a gene of interest, such as gene overexpressed by the targeted stem cell. Exemplary genes to be targeted in the case of BE stem cells are provided in Tables 1-5 and FIGS. 9-11. Small interfering RNA (siRNA) are RNA duplexes normally 21-30 nucleotides long that can associate with a cytoplasmic multi-protein complex known as RNAi-induced silencing complex (RISC). RISC loaded with siRNA mediates the degradation of homologous mRNA transcripts, therefore siRNA can be designed to knock down protein expression with high specificity. A variety of RNAi reagents, including siRNAs targeting clinically relevant targets, are currently under pharmaceutical development, as described, e.g., in de Fougerolles, A. et al., Nature Reviews 6:443-453 (2007).

While the first described RNAi molecules were RNA:RNA hybrids comprising both an RNA sense and an RNA antisense strand, it has now been demonstrated that DNA sense:RNA antisense hybrids, RNA sense:DNA antisense hybrids, and DNA:DNA hybrids are capable of mediating RNAi (Lamberton, J. S. and Christian, A. T., (2003) Molecular Biotechnology 24:111-119). Thus, the invention includes the use of RNAi molecules comprising any of these different types of double-stranded molecules. In addition, it is understood that RNAi molecules may be used and introduced to cells in a variety of forms. Accordingly, as used herein, RNAi molecules encompasses any and all molecules capable of inducing an RNAi response in cells, including, but not limited to, double-stranded polynucleotides comprising two separate strands, i.e. a sense strand and an antisense strand, e.g., small interfering RNA (siRNA); polynucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

RNA interference (RNAi) may be used to specifically inhibit expression of target genes in the stem cell. Double-stranded RNA-mediated suppression of gene and nucleic acid expression may be accomplished according to the invention by introducing dsRNA, siRNA or shRNA into cells or organisms. SiRNA may be double-stranded RNA, or a hybrid molecule comprising both RNA and DNA, e.g., one RNA strand and one DNA strand. It has been demonstrated that the direct introduction of siRNAs to a cell can trigger RNAi in mammalian cells (Elshabir, S. M., et al. Nature 411:494-498 (2001)). Furthermore, suppression in mammalian cells occurred at the RNA level and was specific for the targeted genes, with a strong correlation between RNA and protein suppression (Caplen, N. et al., Proc. Natl. Acad. Sci. USA 98:9746-9747 (2001)).

RNAi molecules targeting specific genes can be readily prepared according to procedures known in the art. Structural characteristics of effective siRNA molecules have been identified. Elshabir, S. M. et al. (2001) Nature 411:494-498 and Elshabir, S. M. et al. (2001), EMBO 20:6877-6888. Accordingly, one of skill in the art would understand that a wide variety of different siRNA molecules may be used to target a specific gene or transcript. In certain embodiments, siRNA molecules according to the invention are double-stranded and 16-30 or 18-25 nucleotides in length, including each integer in between. In one embodiment, an siRNA is 21 nucleotides in length. In certain embodiments, siRNAs have 0-7 nucleotide 3′ overhangs or 0-4 nucleotide 5′ overhangs. In one embodiment, an siRNA molecule has a two nucleotide 3′ overhang. In one embodiment, an siRNA is 21 nucleotides in length with two nucleotide 3′ overhangs (i.e. they contain a 19 nucleotide complementary region between the sense and antisense strands). In certain embodiments, the overhangs are UU or dTdT 3′ overhangs.

Generally, siRNA molecules are completely complementary to the target mRNA molecule, since even single base pair mismatches have been shown to reduce silencing. In other embodiments, siRNAs may have a modified backbone composition, such as, for example, 2′-deoxy- or 2′-O-methyl modifications. However, in preferred embodiments, the entire strand of the siRNA is not made with either 2′ deoxy or 2′-O-modified bases.

In one embodiment, siRNA target sites are selected by scanning the target mRNA transcript sequence for the occurrence of AA dinucleotide sequences. Each AA dinucleotide sequence in combination with the 3′ adjacent approximately 19 nucleotides are potential siRNA target sites. In one embodiment, siRNA target sites are preferentially not located within the 5′ and 3′ untranslated regions (UTRs) or regions near the start codon (within approximately 75 bases), since proteins that bind regulatory regions may interfere with the binding of the siRNP endonuclease complex (Elshabir, S. et al. Nature 411:494-498 (2001); Elshabir, S. et al. EMBO J. 20:6877-6888 (2001)). In addition, potential target sites may be compared to an appropriate genome database, such as BLASTN 2.0.5, available on the NCBI server at www.ncbi.nlm, and potential target sequences with significant homology to other coding sequences eliminated.

Short Hairpin RNA (shRNA) is a form of hairpin RNA capable of sequence-specifically reducing expression of a target gene. Short hairpin RNAs may offer an advantage over siRNAs in suppressing gene expression, as they are generally more stable and less susceptible to degradation in the cellular environment. It has been established that such short hairpin RNA-mediated gene silencing works in a variety of normal and cancer cell lines, and in mammalian cells, including mouse and human cells. Paddison, P. et al., Genes Dev. 16(8):948-58 (2002). Furthermore, transgenic cell lines bearing chromosomal genes that code for engineered shRNAs have been generated. These cells are able to constitutively synthesize shRNAs, thereby facilitating long-lasting or constitutive gene silencing that may be passed on to progeny cells. Paddison, P. et al., Proc. Natl. Acad. Sci. USA 99(3):1443-1448 (2002).

ShRNAs contain a stem loop structure. In certain embodiments, they may contain variable stem lengths, typically from 19 to 29 nucleotides in length, or any number in between. In certain embodiments, hairpins contain 19 to 21 nucleotide stems, while in other embodiments, hairpins contain 27 to 29 nucleotide stems. In certain embodiments, loop size is between 4 to 23 nucleotides in length, although the loop size may be larger than 23 nucleotides without significantly affecting silencing activity. ShRNA molecules may contain mismatches, for example G-U mismatches between the two strands of the shRNA stem without decreasing potency. In fact, in certain embodiments, shRNAs are designed to include one or several G-U pairings in the hairpin stem to stabilize hairpins during propagation in bacteria, for example. However, complementarity between the portion of the stem that binds to the target mRNA (antisense strand) and the mRNA is typically required, and even a single base pair mismatch is this region may abolish silencing. 5′ and 3′ overhangs are not required, since they do not appear to be critical for shRNA function, although they may be present (Paddison et al. (2002) Genes & Dev. 16(8):948-58).

MicroRNAs

In other embodiments, the nucleic acid therapeutic is a Micro RNA (mi RNA), MicroRNA mimic or an antagonist. Micro RNAs (miRNAs) are a highly conserved class of small RNA molecules that are transcribed from DNA in the genomes of plants and animals, but are not translated into protein. Processed miRNAs are single stranded @17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S, NAR, 2004, 32, Database Issue, D109-D111; and also at http://microrna.sanger.ac.uk/sequences/. In certain preferred embodiments, the mi RNA, mi RNA mimic or antagonist is selectively cytotoxic or cytotoxic to BE cell as relative to normal esophageal squamous epithelium and/or esophageal squamous stem cells and/or gastric cardia stem cells.

Antisense Oligonucleotides

In one embodiment, the nucleic acid therapeutic is an antisense oligonucleotide directed to a target gene overexpressed in the stem cell, i.e., the BE stem cell, or for which inhibition of expression is selectively cytotoxic or cytotoxic to the BE cell as relative to normal esophageal squamous epithelium and/or esophageal squamous stem cells and/or stomach cardia stem cells. The term “antisense oligonucleotide” or simply “antisense” is meant to include oligonucleotides that are complementary to a targeted polynucleotide sequence. Antisense oligonucleotides are single strands of DNA or RNA that are complementary to a chosen sequence. In the case of antisense RNA, they prevent translation of complementary RNA strands by binding to it. Antisense DNA can be used to target a specific, complementary (coding or non-coding) RNA. If binding takes places this DNA/RNA hybrid can be degraded by the enzyme RNase H. In particular embodiment, antisense oligonucleotides contain from about 10 to about 50 nucleotides, more preferably about 15 to about 30 nucleotides. The term also encompasses antisense oligonucleotides that may not be exactly complementary to the desired target gene. Thus, the invention can be utilized in instances where non-target specific-activities are found with antisense, or where an antisense sequence containing one or more mismatches with the target sequence is the most preferred for a particular use.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tₘ, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res. 1997, 25(17):3389-402).

Ribozymes

According to another embodiment of the invention, the nucleic acid therapeutic is a ribozyme. Ribozymes are RNA-protein complexes having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20) and can cleave an inactive a target mRNA. For example, a large number of ribozymes accelerate phosphodiester transfer reactions with a high degree of specificity, often cleaving only one of several phosphodiesters in an oligonucleotide substrate (Cech et al., Cell. 1981 December; 27(3 Pt 2):487-96; Michel and Westhof, J. Mol. Biol. 1990 Dec. 5; 216(3):585-610; Reinhold-Hurek and Shub, Nature. 1992 May 14; 357(6374):173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis Δvirus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11; 20(17):4559-65. Examples of hairpin motifs are described by Hampel et al. (Eur. Pat. Appl. Publ. No. EP 0360257), Hampel and Tritz, Biochemistry 1989 Jun. 13; 28(12):4929-33; Hampel et al., Nucleic Acids Res. 1990 Jan. 25; 18(2):299-304 and U.S. Pat. No. 5,631,359. An example of the hepatitis virus motif is described by Perrotta and Been, Biochemistry. 1992 Dec. 1; 31(47):11843-52; an example of the RNaseP motif is described by Guerrier-Takada et al., Cell. 1983 December; 35(3 Pt 2):849-57; Neurospora VS RNA ribozyme motif is described by Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(11):2795-9); and an example of the Group I intron is described in U.S. Pat. No. 4,987,071. Desirable characteristics of enzymatic nucleic acid molecules used according to the invention are that they have a specific substrate binding site which is complementary to one or more of the target RNA regions, and that they have nucleotide sequences within or surrounding that substrate binding site which impart an RNA cleaving activity to the molecule. Thus the ribozyme constructs need not be limited to specific motifs mentioned herein.

Methods of producing a ribozyme targeted to any polynucleotide sequence are known in the art. Ribozymes may be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

Ribozyme activity can be optimized by altering the length of the ribozyme binding arms or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Cell Penetrating Moieties Attached to the Nucleic Acid Therapeutics

A variety of agents can be associated with the nucleic acid therapeutic, preferably through a reversible covalent linker, in order to enhance the uptake of the therapeutic by cells, particularly the targeted stem cell. These cell penetrating (CP) moieties may be so attached directly or indirectly via a linker. Functionally, the CP moieties may be designed to achieve one or more improved outcomes. As used herein the term “CP moiety” is a compound or molecule or construct which is attached, linked or associated with the nucleic acid therapeutic.

In one embodiment the CP moieties comprise molecules which promote endocytosis of the nucleic acid therapeutic. As such the CP moiety acts as a “membrane intercalator.” For example, the membrane intercalators may comprise C₁₀-C₁₈ moieties which may be attached to the 3′ end of antisense strand. These moieties may facilitate or result in the nucleic acid therapeutic becoming embedded in the lipid bilayer of a cell. Upon “flipping” of the lipids, the nucleic acid therapeutic would then enter the cell. In these constructs, the linker between the CP moiety and the nucleic acid therapeutic can be selected such that it is sensitive to the physicochemical environment of the cell and/or to be susceptible to or resistant to enzymes present. The end result being the liberation of the nucleic acid therapeutic, with or without a portion of the optional linker. The present invention also contemplates nucleic acid therapeutics that bind to receptors which are internalized.

Furthermore, the nucleic acid therapeutics of the invention itself can have one or more CP moieties which facilitates the active or passive transport, localization, or compartmentalization of the nucleic acid therapeutic.

Conjugates as CP Moieties

CP moieties, while attached directly to the nucleic acid therapeutic or to the nucleic acid therapeutic via an optional linker may comprise conjugate groups attached to one or more of the nucleic acid therapeutic termini at selected nucleobase positions, sugar positions or to one of the terminal internucleoside linkages.

There are numerous methods for preparing conjugates of nucleic acid therapeutics. Generally, a nucleic acid therapeutic is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic. For example, an electrophilic group can be a carbonyl-containing functionality and a nucleophilic group can be an amine or thiol. Methods for conjugation of nucleic acids and related compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In some embodiments, conjugate moieties can be attached to the terminus of a nucleic acid therapeutic such as a 5′ or 3′ terminal residue of either strand. Conjugate moieties can also be attached to internal residues of the oligomeric compounds. For nucleic acid therapeutics, conjugate moieties can be attached to one or both strands. In some embodiments, a double-stranded nucleic acid therapeutic contains a conjugate moiety attached to each end of the sense strand. In other embodiments, a double-stranded nucleic acid therapeutic contains a conjugate moiety attached to both ends of the antisense strand.

In some embodiments, conjugate moieties can be attached to heterocyclic base moieties (e.g., purines and pyrimidines), monomeric subunits (e.g., sugar moieties), or monomeric subunit linkages (e.g., phosphodiester linkages) of nucleic acid molecules. Conjugation to purines or derivatives thereof can occur at any position including, endocyclic and exocyclic atoms. In some embodiments, the 2-, 6-, 7-, or 8-positions of a purine base are attached to a conjugate moiety. Conjugation to pyrimidines or derivatives thereof can also occur at any position. In some embodiments, the 2-, 5-, and 6-positions of a pyrimidine base can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms.

Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

These CP moieties act to enhance the properties of the nucleic acid therapeutic or may be used to track the nucleic acid therapeutic or its metabolites and/or effect the trafficking of the construct. Properties that are typically enhanced include without limitation activity, cellular distribution and cellular uptake. In one embodiment, the nucleic acid therapeutics are prepared by covalently attaching the CP moieties to chemically functional groups available on the nucleic acid therapeutic or linker such as hydroxyl or amino functional groups. Conjugates which may be used as terminal moities include intercalators, reporter molecules, polyamines, polyamides, polyethylene glycols, polyethers, and groups that enhance the pharmacodynamic and/or pharmacokinetic properties of the nucleic acid therapeutic.

Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve properties including but not limited to construct uptake, construct resistance to degradation, and/or strengthen sequence-specific hybridization with RNA.

Conjugate groups also include but are not limited to lipid moieties such as a cholesterol moiety, cholic acid, a thioether, an aliphatic chain, a phospholipid, a polyamine or a polyethylene glycol chain or adamantane acetic acid, a palmityl moiety or an octadecylamine or hexylamino-carbonyl-oxycholesterol moiety.

The nucleic acid therapeutics of the invention may also be conjugated to active drug substances. Representative U.S. patents that teach the preparation of such conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The present invention provides, inter alia, nucleic acid therapeutics and compositions containing the same wherein the CP moiety comprises one or more conjugate moieties. The CP moieties (e.g., conjugates) of the present invention can be covalently attached, optionally through one or more linkers, to one or more nucleic acid therapeutics. The resulting constructs can have modified or enhanced pharmacokinetic, pharmacodynamic, and other properties compared with non-conjugated constructs. A conjugate moiety that can modify or enhance the pharmacokinetic properties of a nucleic acid therapeutic can improve cellular distribution, bioavailability, metabolism, excretion, permeability, and/or cellular uptake of the nucleic acid therapeutic. A conjugate moiety that can modify or enhance pharmacodynamic properties of a nucleic acid therapeutic can improve activity, resistance to degradation, sequence-specific hybridization, uptake, and the like.

Representative conjugate moieties can include lipophilic molecules (aromatic and non-aromatic) including steroid molecules; proteins (e.g., antibodies, enzymes, serum proteins); peptides; vitamins (water-soluble or lipid-soluble); polymers (water-soluble or lipid-soluble); small molecules including drugs, toxins, reporter molecules, and receptor ligands; carbohydrate complexes; nucleic acid cleaving complexes; metal chelators (e.g., porphyrins, texaphyrins, crown ethers, etc.); intercalators including hybrid photonuclease/intercalators; crosslinking agents (e.g., photoactive, redox active), and combinations and derivatives thereof. Oligonucleotide conjugates and their syntheses are also reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense & Nucleic Acid Drug Development, 2002, 12, 103, each of which is incorporated herein by reference in its entirety.

Lipophilic conjugate moieties can be used, for example, to counter the hydrophilic nature of a nucleic acid therapeutic and enhance cellular penetration. Lipophilic moieties include, for example, steroids and related compounds such as cholesterol (U.S. Pat. No. 4,958,013 and Letsinger et al., Proc. Natl. Acad. Sci. USA, 1989, 86, 6553), thiocholesterol (Oberhauser et al., Nuc. Acids Res., 1992, 20, 533), lanosterol, coprostanol, stigmasterol, ergosterol, calciferol, cholic acid, deoxycholic acid, estrone, estradiol, estratriol, progesterone, stilbestrol, testosterone, androsterone, deoxycorticosterone, cortisone, 17-hydroxycorticosterone, their derivatives, and the like.

Other lipophilic conjugate moieties include aliphatic groups, such as, for example, straight chain, branched, and cyclic alkyls, alkenyls, and alkynyls. The aliphatic groups can have, for example, 5 to about 50, 6 to about 50, 8 to about 50, or 10 to about 50 carbon atoms. Example aliphatic groups include undecyl, dodecyl, hexadecyl, heptadecyl, octadecyl, nonadecyl, terpenes, bornyl, adamantyl, derivatives thereof and the like. In some embodiments, one or more carbon atoms in the aliphatic group can be replaced by a heteroatom such as O, S, or N (e.g., geranyloxyhexyl). Further suitable lipophilic conjugate moieties include aliphatic derivatives of glycerols such as alkylglycerols, bis(alkyl)glycerols, tris(alkyl)glycerols, monoglycerides, diglycerides, and triglycerides. Saturated and unsaturated fatty functionalities, such as, for example, fatty acids, fatty alcohols, fatty esters, and fatty amines, can also serve as lipophilic conjugate moieties. In some embodiments, the fatty functionalities can contain from about 6 carbons to about 30 or about 8 to about 22 carbons. Example fatty acids include, capric, caprylic, lauric, palmitic, myristic, stearic, oleic, linoleic, linolenic, arachidonic, eicosanoic acids and the like.

In further embodiments, lipophilic conjugate groups can be polycyclic aromatic groups having from 6 to about 50, 10 to about 50, or 14 to about 40 carbon atoms. Example polycyclic aromatic groups include pyrenes, purines, acridines, xanthenes, fluorenes, phenanthrenes, anthracenes, quinolines, isoquinolines, naphthalenes, derivatives thereof and the like.

Other suitable lipophilic conjugate moieties include menthols, trityls (e.g., dimethoxytrityl (DMT)), phenoxazines, lipoic acid, phospholipids, ethers, thioethers (e.g., hexyl-5-tritylthiol), derivatives thereof and the like. nucleic acid therapeutics containing conjugate moieties with affinity for low density lipoprotein (LDL) can help provide an effective targeted delivery system. High expression levels of receptors for LDL on tumor cells makes LDL an attractive carrier for selective delivery of drugs to these cells (Rump et al., Bioconjugate Chem. 9: 341, 1998; Firestone, Bioconjugate Chem. 5: 105, 1994; Mishra et al., Biochim. Biophys. Acta 1264: 229, 1995). Moieties having affinity for LDL include many lipophilic groups such as steroids (e.g., cholesterol), fatty acids, derivatives thereof and combinations thereof. In some embodiments, conjugate moieties having LDL affinity can be dioleyl esters of cholic acids such as chenodeoxycholic acid and lithocholic acid.

Conjugate moieties can also include vitamins. Vitamins are known to be transported into cells by numerous cellular transport systems. Typically, vitamins can be classified as water soluble or lipid soluble. Water soluble vitamins include thiamine, riboflavin, nicotinic acid or niacin, the vitamin B₆ pyridoxal group, pantothenic acid, biotin, folic acid, the B₁₂ cobamide coenzymes, inositol, choline and ascorbic acid. Lipid soluble vitamins include the vitamin A family, vitamin D, the vitamin E tocopherol family and vitamin K (and phytols).

In some embodiments, the conjugate moiety includes folic acid (folate) and/or one or more of its various forms, such as dihydrofolic acid, tetrahydrofolic acid, folinic acid, pteropolyglutamic acid, dihydrofolates, tetrahydrofolates, tetrahydropterins, 1-deaza, 3-deaza, 5-deaza, 8-deaza, 10-deaza, 1,5-dideaza, 5,10-dideaza, 8,10-dideaza and 5,8-dideaza folate analogs, and antifolates.

Vitamin conjugate moieties include, for example, vitamin A (retinol) and/or related compounds. The vitamin A family (retinoids), including retinoic acid and retinol, are typically absorbed and transported to target tissues through their interaction with specific proteins such as cytosol retinol-binding protein type II (CRBP-II), retinol binding protein (RBP), and cellular retinol-binding protein (CRBP). The vitamin A family of compounds can be attached to a nucleic acid therapeutic via acid or alcohol functionalities found in the various family members. For example, conjugation of an N-hydroxy succinimide ester of an acid moiety of retinoic acid to an amine function on a linker pendant to a nucleic acid therapeutic can result in linkage of vitamin A compound to the nucleic acid therapeutic via an amide bond. Also, retinol can be converted to its phosphoramidite, which is useful for 5′ conjugation.

alpha-Tocopherol (vitamin E) and the other tocopherols (beta through zeta) can be conjugated to nucleic acid therapeutics to enhance uptake because of their lipophilic character. Also, vitamin D, and its ergosterol precursors, can be conjugated to nucleic acid therapeutics through their hydroxyl groups by first activating the hydroxyl groups to, for example, hemisuccinate esters. Conjugation can then be effected directly to the nucleic acid therapeutic or to an amino linker pendant from the nucleic acid therapeutic. Other vitamins that can be conjugated to nucleic acid therapeutics in a similar manner on include thiamine, riboflavin, pyridoxine, pyridoxamine, pyridoxal, deoxypyridoxine. Lipid soluble vitamin K's and related quinone-containing compounds can be conjugated via carbonyl groups on the quinone ring. The phytol moiety of vitamin K can also serve to enhance binding of the oligomeric compounds to cells.

Pyridoxal (vitamin B₆) has specific B₆-binding proteins. Other pyridoxal family members include pyridoxine, pyridoxamine, pyridoxal phosphate, and pyridoxic acid. Pyridoxic acid, niacin, pantothenic acid, biotin, folic acid and ascorbic acid can be conjugated to nucleic acid therapeutics, for example, using N-hydroxysuccinimide esters that are reactive with amino linkers located on the nucleic acid therapeutic, as described above for retinoic acid.

Conjugate moieties can also include polymers. Polymers can provide added bulk and various functional groups to affect permeation, cellular transport, and localization of the conjugated nucleic acid therapeutic. For example, increased hydrodynamic radius caused by conjugation of a nucleic acid therapeutic with a polymer can help prevent entry into the nucleus and encourage localization in the cytoplasm. In some embodiments, the polymer does not substantially reduce cellular uptake or interfere with hybridization to a complementary strand or other target. In further embodiments, the conjugate polymer moiety has, for example, a molecular weight of less than about 40, less than about 30, or less than about 20 kDa. Additionally, polymer conjugate moieties can be water-soluble and optionally further comprise other conjugate moieties such as peptides, carbohydrates, drugs, reporter groups, or further conjugate moieties.

In some embodiments, polymer conjugates include polyethylene glycol (PEG) and copolymers and derivatives thereof. Conjugation to PEG has been shown to increase nuclease stability of nucleic acid based compounds. PEG conjugate moieties can be of any molecular weight including for example, about 100, about 500, about 1000, about 2000, about 5000, about 10,000 and higher. In some embodiments, the PEG conjugate moieties contains at least 4, at least 5, at least 6, at least 7, at least 8, at least 9, at least 10, at least 15, at least 20, or at least 25 ethylene glycol residues. In further embodiments, the PEG conjugate moiety contains from about 4 to about 10, about 4 to about 8, about 5 to about 7, or about 6 ethylene glycol residues. The PEG conjugate moiety can also be modified such that a terminal hydroxyl is replaced by alkoxy, carboxy, acyl, amido, or other functionality. Other conjugate moieties, such as reporter groups including, for example, biotin or fluorescein can also be attached to a PEG conjugate moiety. Copolymers of PEG are also suitable as conjugate moieties. Preparation and biological activity of polyethylene glycol conjugates of oligonucleotides are described, for example, in Bonora et al., Nucleosides Nucleotides 18: 1723, 1999; Bonora et al., Farmaco 53: 634, 1998; Efimov, Bioorg. Khim. 19: 800, 1993; and Jaschke et al., Nucleic Acids Res. 22: 4810, 1994. Further example PEG conjugate moieties and preparation of corresponding conjugated oligomeric compounds is described in, for example, U.S. Pat. Nos. 4,904,582 and 5,672,662, each of which is incorporated by reference herein in its entirety. Nucleic acid compounds conjugated to one or more PEG moieties are available commercially.

Other polymers suitable as conjugate moieties include polyamines, polypeptides, polymethacrylates (e.g., hydroxylpropyl methacrylate (HPMA)), poly(L-lactide), poly(DL lactide-co-glycolide (PGLA), polyacrylic acids, polyethylenimines (PEI), polyalkylacrylic acids, polyurethanes, polyacrylamides, N-alkylacrylamides, polyspermine (PSP), polyethers, cyclodextrins, derivatives thereof and co-polymers thereof. Many polymers, such as PEG and polyamines have receptors present in certain cells, thereby facilitating cellular uptake. Polyamines and other amine-containing polymers can exist in protonated form at physiological pH, effectively countering an anionic backbone of some oligomeric compounds, effectively enhancing cellular permeation. Some example polyamines include polypeptides (e.g., polylysine, polyomithine, polyhistadine, polyarginine, and copolymers thereof), triethylenetetramine, spermine, polyspermine, spermidine, synnorspermidine, C-branched spermidine, and derivatives thereof. Other amine-containing moieties can also serve as suitable conjugate moieties due to, for example, the formation of cationic species at physiological conditions. Example amine-containing moieties include 3-aminopropyl, 3-(N,N-dimethylamino)propyl, 2-(2-(N,N-dimethylamino)ethoxy)ethyl, 2-N-(2-aminoethyl)-N-methylaminooxy)ethyl, 2-(1-imidazolyl)ethyl, and the like.

Conjugate moieties can also include peptides. Suitable peptides can have from 2 to about 30, 2 to about 20, 2 to about 15, or 2 to about 10 amino acid residues. Amino acid residues can be naturally or non-naturally occurring, including both D and L isomers.

In some embodiments, peptide conjugate moieties are pH sensitive peptides such as fusogenic peptides. Fusogenic peptides can facilitate endosomal release of agents such as nucleic acid therapeutics to the cytoplasm.

It is believed that fusogenic peptides change conformation in acidic pH, effectively destabilizing the endosomal membrane thereby enhancing cytoplasmic delivery of endosomal contents. Example fusogenic peptides include peptides derived from polymyxin B, influenza HA2, GAL4, KALA, EALA, melittin-derived peptide, .alpha.-helical peptide or Alzheimer .beta.-amyloid peptide, and the like. Preparation and biological activity of oligonucleotides conjugated to fusogenic peptides are described in, for example, Bongartz et al., Nucleic Acids Res. 22: 4681, 1994, and U.S. Pat. Nos. 6,559,279 and 6,344,436.

Other peptides that can serve as conjugate moieties include delivery peptides which have the ability to transport relatively large, polar molecules (including peptides, oligonucleotides, and proteins) across cell membranes. Example delivery peptides include Tat peptide from HIV Tat protein and Ant peptide from Drosophila antenna protein. Conjugation of Tat and Ant with oligonucleotides is described in, for example, Astriab-Fisher et al., Biochem. Pharmacol. 60: 83, 2000.

Conjugated delivery peptides can help control localization of nucleic acid therapeutics and constructs to specific regions of a cell, including, for example, the cytoplasm, nucleus, nucleolus, and endoplasmic reticulum (ER). Nuclear localization can be effected by conjugation of a nuclear localization signal (NLS). In contrast, cytoplasmic localization can be facilitated by conjugation of a nuclear export signal (NES). Methods for conjugating peptides to oligomeric compounds such as oligonucleotides is described in, for example, U.S. Pat. No. 6,559,279, which is incorporated herein by reference in its entirety.

Many drugs, receptor ligands, toxins, reporter molecules, and other small molecules can serve as conjugate moieties. Small molecule conjugate moieties often have specific interactions with certain receptors or other biomolecules, thereby allowing targeting of conjugated nucleic acid therapeutics to specific cells or tissues.

Other conjugate moieties can include proteins, subunits, or fragments thereof. Proteins include, for example, enzymes, reporter enzymes, antibodies, receptors, and the like. In some embodiments, protein conjugate moieties can be antibodies or fragments. Antibodies can be designed to bind to desired targets such as tumor and other disease-related antigens. In further embodiments, protein conjugate moieties can be serum proteins. In yet further embodiments, nucleic acid therapeutics can be conjugated to RNAi-related proteins, RNAi-related protein complexes, subunits, and fragments thereof. For example, oligomeric compounds can be conjugated to Dicer or RISC or fragments thereof. RISC is a ribonucleoprotein complex that contains an oligonucleotide component and proteins of the Argonaute family of proteins, among others. Argonaute proteins make up a highly conserved family whose members have been implicated in RNA interference and the regulation of related phenomena. Members of this family have been shown to possess the canonical PAZ and Piwi domains, thought to be a region of protein-protein interaction. Other proteins containing these domains have been shown to effect target cleavage, including the RNAse, Dicer.

Other conjugate moieties can include, for example, oligosaccharides and carbohydrate clusters; a glycotripeptide that binds to GaI/GaINAc receptors on hepatocytes, lysine-based galactose clusters; and cholane-based galactose clusters (e.g., carbohydrate recognition motif for asialoglycoprotein receptor). Further suitable conjugates can include oligosaccharides that can bind to carbohydrate recognition domains (CRD) found on the asialoglycoprotein-receptor (ASGP-R).

A wide variety of linker groups are known in the art that can be useful in the attachment of CP moieties to nucleic acid therapeutics. A review of many of the useful linker groups can be found in, for example, Antisense Research and Applications, S. T. Crooke and B. Lebleu, Eds., CRC Press, Boca Raton, Fla., 1993, p. 303-350. Any of the reported groups can be used as a single linker or in combination with one or more further linkers.

Linkers and their use in preparation of conjugates of oligonucleotides are provided throughout the art. For example, see U.S. Pat. Nos. 4,948,882; 5,525,465; 5,541,313; 5,545,730; 5,552,538; 5,580,731; 5,486,603; 5,608,046; 4,587,044; 4,667,025; 5,254,469; 5,245,022; 5,112,963; 5,391,723; 5,510,475; 5,512,667; 5,574,142; 5,684,142; 5,770,716; 6,096,875; 6,335,432; and 6,335,437.

In one embodiment, the linker may comprise a nucleic acid hairpin which links the 5′ end of one strand

The term “linking moiety,” or “linker” as used herein is generally a bi-functional group, molecule or compound. It may covalently or non-covalently bind the nucleic acid therapeutic to the CP moiety. The covalent binding may be at both or only one end of the linker. Whether the nature of binding to the nucleic acid therapeutic and CP moiety is either covalent or noncovalent, the linker itself may be labile. As used herein the term “labile” as it applies to linkers means that the linker is either temporally or spatially stable for only a definite period or under certain environmental conditions. For example, a labile linker may lose integrity at a certain, time, temperature, pH, pressure, or under a certain magnetic field or electric field. The result of lost integrity being the severance of the connection between the nucleic acid therapeutic and one or more CP moieties.

Suitable linking moieties or linkers include, but are not limited to, divalent group such as alkylene, cycloalkylene, arylene, heterocyclyl, heteroarylene, and the other variables are as described herein.

### C. Imaging Methods

In another aspect, the invention provides methods for imaging metaplasia (e.g., esophageal metaplasia). The methods of the invention generally comprise administering to a subject an effective amount of an agent that specifically binds to a cell surface polypeptide encoded by one of the genes set forth in Tables 1-5, 15 and 16 and FIGS. 9-11, and visualizing the agent. In a preferred embodiment, cell surface proteins are used that are differentially expressed in Barrett's esophagus progenitor cells and squamous cell progenitor cells and/or gastric cardia progenitor cells.

Any agent that binds to the desired cell surface polypeptide is suitable for use in the methods of the invention. Suitable agents include, without limitation, antibodies, aptamers, peptides, cell surface receptor ligands, or small molecules. In a preferred embodiment, the agent is an antibody, antibody-like molecule or cell surface receptor ligand.

In some embodiments, the agent is linked (covalently or non-covalently) to an imaging moiety to facilitate detection of the agent. Any imaging moiety is suitable for use in the methods of the invention, including, without limitation, positron-emitters, nuclear magnetic resonance spin probes, an optically visible dye, or an optically visible particle. Suitable positron-emitters include, without limitation, positron emitters of oxygen, nitrogen, iron, carbon, or gallium, ⁴³K, ⁵²Fe, ⁵⁷Co, ⁶⁷Cu, ⁶⁷Ga, ⁶⁶Ga, ¹²³I, ¹²⁵I, ¹³¹I, ¹³², or ⁹⁹Tc. Suitable nuclear magnetic resonance spin probes include, without limitation, iron chelates and radioactive chelates of gadolinium or manganese.

In certain embodiments, abalation techniques are used in conjunction with imaging methods disclosed herein. For example, the expression markers described herein may improve the ability to image or otherwise visualize metaplastic cells and facilitate their ablation. The types of ablation technique that techniques that be used in conjunction with imaging or other visualization of markers described herein include radiofrequency, laser, microwave, cryogenic, thermal, chemical, and the like. The ablation probe may conform to the ablation energy source. For example, an endoscope with fiber optics can be used to view the operation field, and to help select the areas for ablation based on the detection of one or more markers described here.

### D. Diagnostic Methods

In another aspect, the invention provides methods for diagnosing, or predicting the future development of metaplasia (e.g., esophageal metaplasia). The methods of the invention generally comprise measuring the expression level of one or more of the genes set forth in Tables 1-5, 15 and 16 and FIGS. 9-11 in an epithelial tissue sample from a subject, wherein an increase in the expression level relative to a suitable control indicates that the subject has, or has a future risk of developing, metaplasia. In a preferred embodiment, cell surface proteins are used that are differentially expressed in Barrett's esophagus progenitor cells and squamous cell progenitor cells.

Any means for measuring the expression level of a gene is suitable for use in the methods of the invention. Exemplary, art recognized, methods include, without limitation, gene expression profiling using gene chips to detect mRNA levels or antibody-based binding assays (e.g. ELISA) to detect the protein-product of a gene.

The epithelial tissue sample can be obtained by any means, including biopsy or by scraping or swabbing an area or by using a needle to aspirate. Methods for collecting various body samples are well known in the art, including, without limitation, endoscopic biopsy. Tissue samples may be fresh, frozen, or fixed according to methods known to one of skill in the art.

The diagnostic methods of the invention are generally performed in vitro. However, in certain embodiments, the tissue sample is not excised, but instead, assayed in vivo, for example, by using agents that can measure the real-time levels of a gene or gene product in the patient's tissue.

In certain embodiments, those patients that have been determined to be at risk of developing metaplasia and are at high degree of risk of developing cancer can then be selected for prophylactic treatment. In exemplary embodiments, the epithelial stem cell crypts that give rise to the metaplasia can be proactively and selectively ablated, such as using techniques described above, before any occurrence of transformed cells or development of esophageal or other cancers.

### E. Screening Methods

In another aspect, the invention provides methods of identifying a compound useful for treating esophageal metaplasia (e.g., esophageal metaplasia).

In one embodiment, the method generally comprises administering a test compound to a p63 null mouse and determining the amount of epithelial metaplasia in the presence and absence of the test compound, wherein a decrease in the amount of epithelial metaplasia identifies a compound useful for treating esophageal metaplasia.

Suitable p63 null mice include mice with complete germ-line deletion of the p63 gene (see e.g., Yang et al. Nature 1999; 398: 714-8), mice in which the p63 gene has been conditionally deleted in one or more epithelial tissue, and mice in which the cellular levels of p63 protein have been reduced (e.g., by RNAi-mediated gene silencing).

In another embodiment, the method generally comprises administering a test compound to a mouse, wherein the mouse comprises stratified epithelial tissue in which basal cells have been ablated, and determining the amount of epithelial metaplasia in said epithelial tissue in the presence and absence of the test compound, wherein a decrease in the amount of epithelial metaplasia identifies a compound useful for treating esophageal metaplasia.

The basal cells of the mouse stratified epithelial tissue can be ablated using any art-recognized means. In a preferred embodiment, basal cells are ablated using Cre-mediated expression of diphtheria toxin fragment A as described in Ivanova et al. Genesis. 2005; 43:129-35.

The amount of epithelial metaplasia can be determined by any means, including by the examination of pathological specimens obtained from sacrificed mice.

The test compound can be administered to the mice by any route and means that will achieve delivery of the test compound to the requisite location.

In another embodiment, the method generally comprises administering a test compound to a Barrett's esophagus progenitor cell, wherein in the presence and absence of the test compound, wherein a decrease in the viability of the Barrett's esophagus progenitor cell identifies a compound useful for treating esophageal metaplasia. The reduction in viability can be a 50, 60, 70, 75, 80, 85, 90, 95, 96, 97, 98, 99 or 100% reduction in viability.

### IV. Exemplification

The invention now being generally described, it will be more readily understood by reference to the following examples, which are included merely for purposes of illustration of certain aspects and embodiments of the present invention, and are not intended to limit the invention.

### General Methods

In general, the practice of the present invention employs, unless otherwise indicated, conventional techniques of chemistry, molecular biology, recombinant DNA technology, immunology (especially, e.g., immunoglobulin technology), and animal husbandry. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al, C.S.H.L. Press, Pub. (1999); Current Protocols in Molecular Biology, eds. Ausubel et al., John Wiley & Sons (1992).

### Animal Models

p63−/− mice used in this study were backcrossed 10-12 times on a BALB/c background (Yang et al., 1999 supra). Wild type controls were derived from littermates. To obtain staged embryos, heterozygotes were crossed and the presence of vaginal plugs set the timing at E0.5. The heterozygous DTA-Krt14-Cre strain was generated by crossing the homozygous Gt(ROSA)26Sor<tm1(DTA)Jpmb>/J stain (Ivanova et al. Genesis. 2005; 43:129-35. (Jackson Laboratory) with the homozygous Tg(KRT14-cre/Esr1)20Efu/J (see Vasioukhin et al. Proc Natl Acad Sci USA. 1999; 96:8551-6) (Jackson laboratory). Diptheria toxin A was transcriptionally activated in basal cells of stratified epithelia via intraperitoneal injection of Tamoxifen in corn oil (100 mg/kg) one to three weeks prior to analysis. Porcine gastroesophageal junctions of three-month-old pigs were obtained from a local abattoir in Strasbourg. Human gastrointestinal junctions were obtained from autopsies at the Brigham and Women's Hospital under IRB approval.

### Expression Microarrays and Bioinformatics

All Cel files were processed using GeneChip Operating Software to calculate probeset intensity values, and probe hybridization ratios were calculated using Affymetrix Expression Console Software to valid sample quality. These intensity values were log 2 transformed and then imported into Partek Genomics Suite 6.5 (beta). A 1-way ANOVA was performed to identify differentially expressed genes. For each analysis, fold-changes and p-values for probesets were calculated. Principal component analysis (PCA) was carried out using all probesets, and heatmaps were generated using sorted datasets based on Euclidean distance and average linkage methods.

Gene expression datasets from normal and Barrett's esophagus were downloaded from the Gene Expression Omnibus (GEO) Genesets of the NCBI (Stairs et al. PLoS One. 2008; 3:e3534). Barrett's metaplasia datasets containing considerable squamous gene expression were excluded from the analysis.

### Histology and Immunofluorescence

Histology, immunohistochemistry, and immunofluorescence were performed using standard techniques. Details on the primary and secondary antibodies employed in these studies are detailed in the Appendix.

### Example 1

### Gastric and Esophageal Metaplasia in the p63 Null Mouse is Similar to that Seen in Barrett's Metaplasia

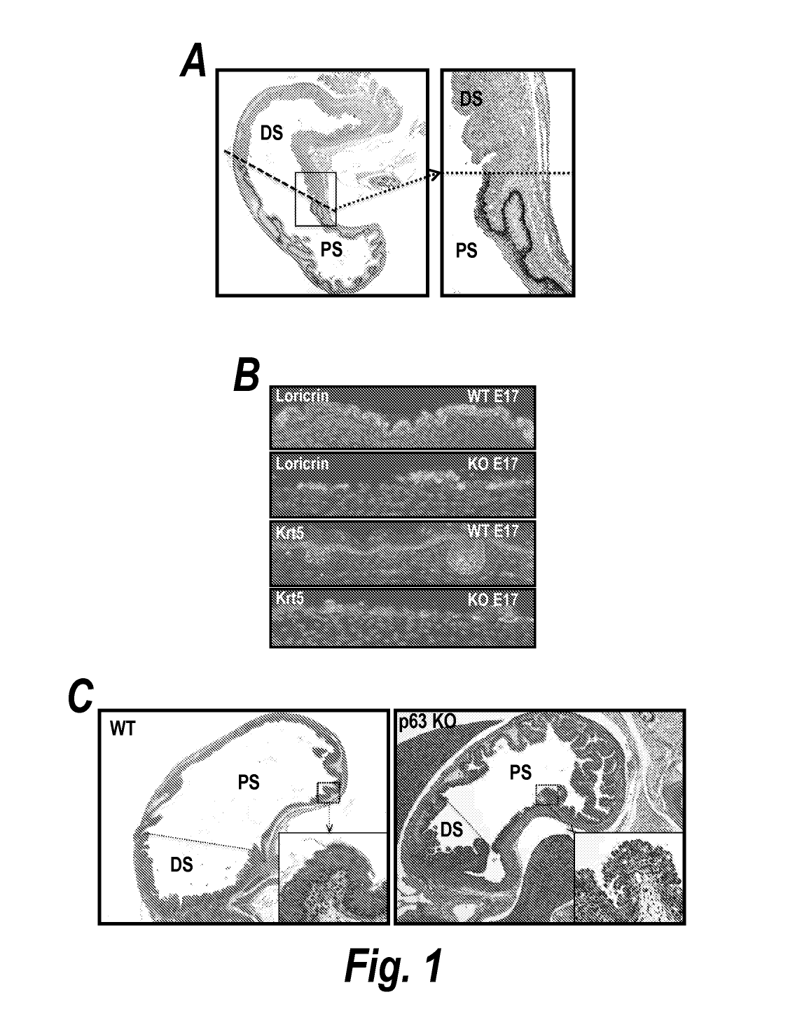

The squamocolumnar junction present at the distal esophagus in humans is shifted posteriorly in mice due to an extension of squamous epithelium to the gastric midline (FIG. 1a). As with all stratified epithelia, the p63 gene is expressed in the basal cells of the esophageal and gastric squamous epithelia (Yang et al. Mol. Cell. 1998; 2:305-16) (FIG. 1a). In p63 null mice, embryos develop to term but are born without an epidermis, mammary or prostate glands, and virtually all other stratified epithelial are either absent or highly deranged (Yang et al. 1999, supra). The epidermis, for instance, begins its normal stratification from a single layer of ectoderm at embryonic day 13-14 (E13-14) and by E17 is a squamous epithelium with suprabasal expression of differentiation markers such as loricrin (FIG. 1b). However, the p63 null epidermis begins to degrade from that point on as evidenced by discontinuous loricrin and keratin 5 staining (FIG. 1b) in a process of non-regenerative differentiation due to the depletion of stem cells (Senoo et al. Cell. 2007; 129:523-36). To determine if similar events occur in the squamous epithelia of the esophagus and proximal stomach of p63 null embryos, these regions were examined by histologically. Although the wild type E18 embryo shows a mature squamous epithelium in the proximal stomach (FIG. 1c), the p63 null embryo showed a remarkably well-developed columnar epithelium marked by hobnail apical projections (FIG. 1d). Taken together these data demonstrate that gastric and esophageal metaplasia in the p63 Null Mouse is similar to that seen in Barrett's metaplasia.

### Example 2

### Gene Expression of Metaplasia the in p63 Null Mouse is Similar to that Seen in Barrett's Metaplasia

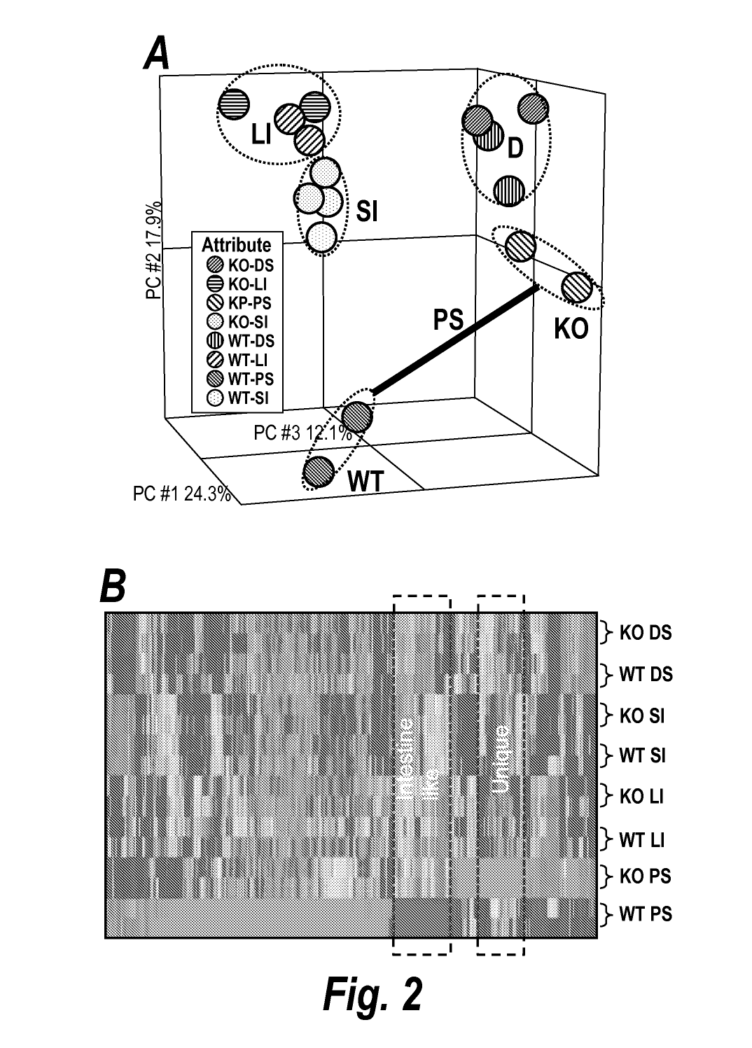

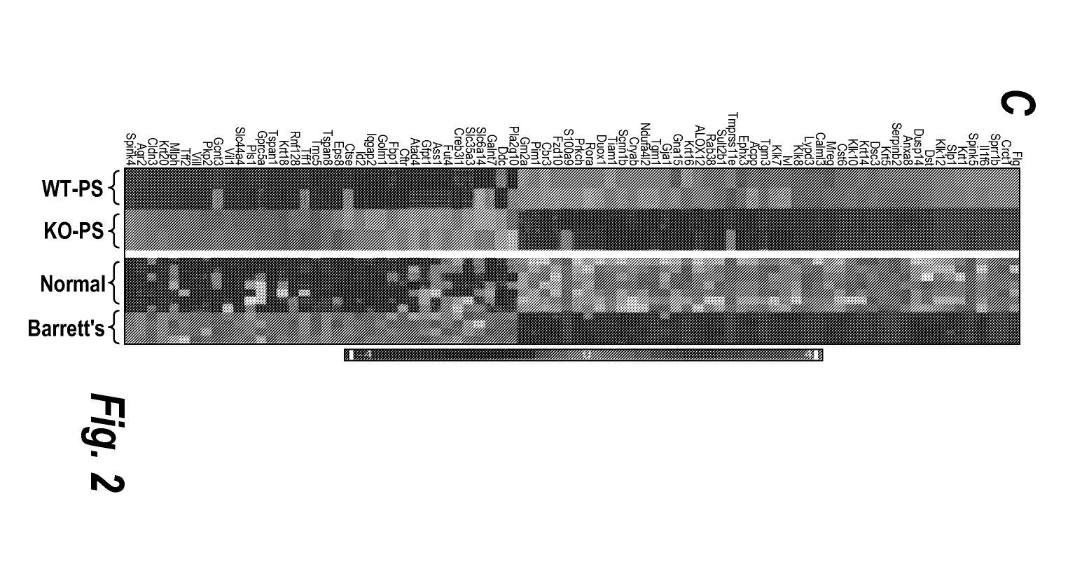

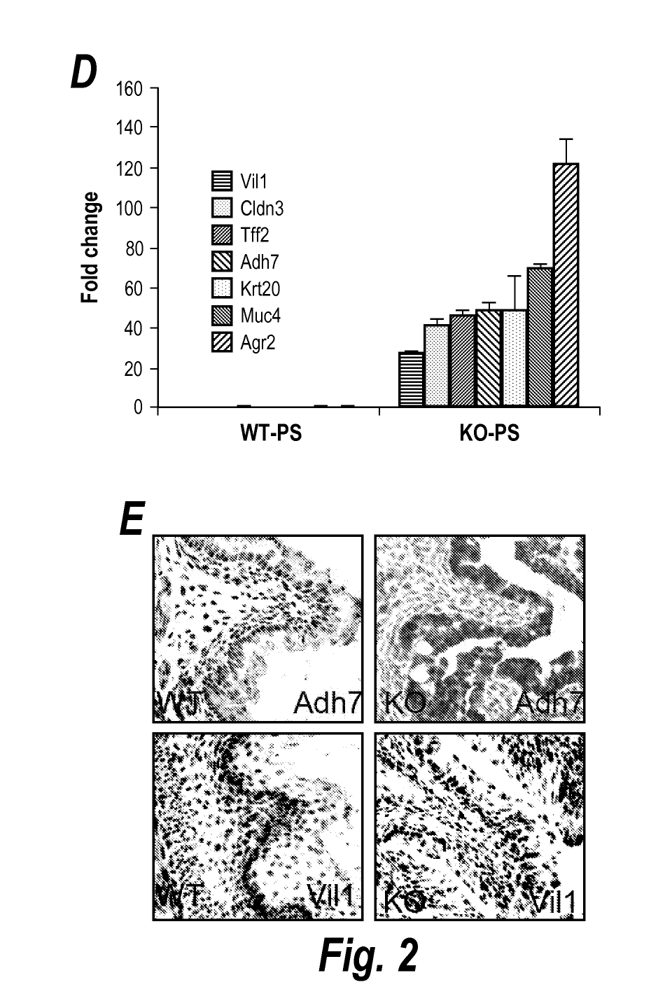

To more fully characterize the metaplasia in the proximal stomach of the p63 null embryo, its gene expression profile was compared with those of specific regions of the gastrointestinal tract in mutant and wild type animals. In brief, RNA was extracted from microdissected tissues and used to probe expression microarray chips (Mouse Genome 430 2.0 Array, Affymetrix). Unsupervised principal component analysis of these data revealed that the wild type and p63 null colon, small intestine, and distal stomach formed concordant pairs of overall gene expression (FIGS. 2a, 2b). In contrast, the comparisons of gene expression between wild type and p63 null proximal stomach revealed stark differences, thus the observed metaplasia was clearly distinct from the indigenous squamous epithelia at this site. Moreover, a broad comparison of the gene expression profiles of the metaplasia in the p63 null embryos indicated only passing relationships with either the small or large intestines (FIG. 2b, “intestine-like” box), demonstrating that this metaplasia is much more an entity unto itself rather than of other major tissues of the gastrointestinal tract. The gene expression profile of the metaplasia in the p63 null embryo was then compared with available datasets (Stairs et al. 2008 supra) from the intestinal metaplasia of human Barrett's esophagus (FIG. 2c). Within the top fifty genes overrepresented in the metaplasia of the p63 null embryos were many of the markers established for Barrett's and gastric intestinal metaplasia (Wang et al. J Gastroenterol 2009; 44:897-911), including mucin 4 (73×), keratin 20 (61×), trefoil factor 2 (49×), claudin 3 (46×), Agr2 (120×), and villin (27×; p<10⁻⁷ for all) (FIG. 2d). Moreover, antibodies to multiple markers, including Adh7 and Agr2, showed robust staining of the proximal stomach of the mutant embryos (FIG. 2e), validating the relevance of these expression datasets to the observed metaplasia. Taken together these data demonstrate that gene expression of metaplasia the in p63 null mouse is similar to that seen in Barrett's metaplasia

### Example 3

### Metaplasia Evolves from a Car-4-Positive, Primitive Embryonic Epithelium

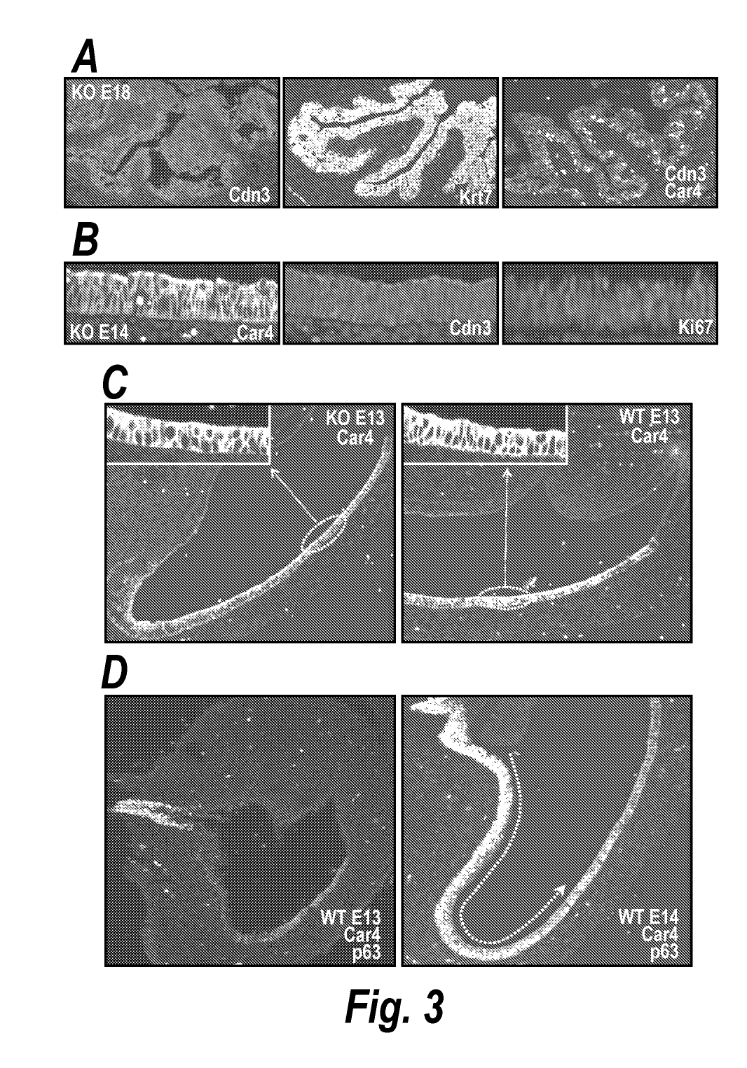

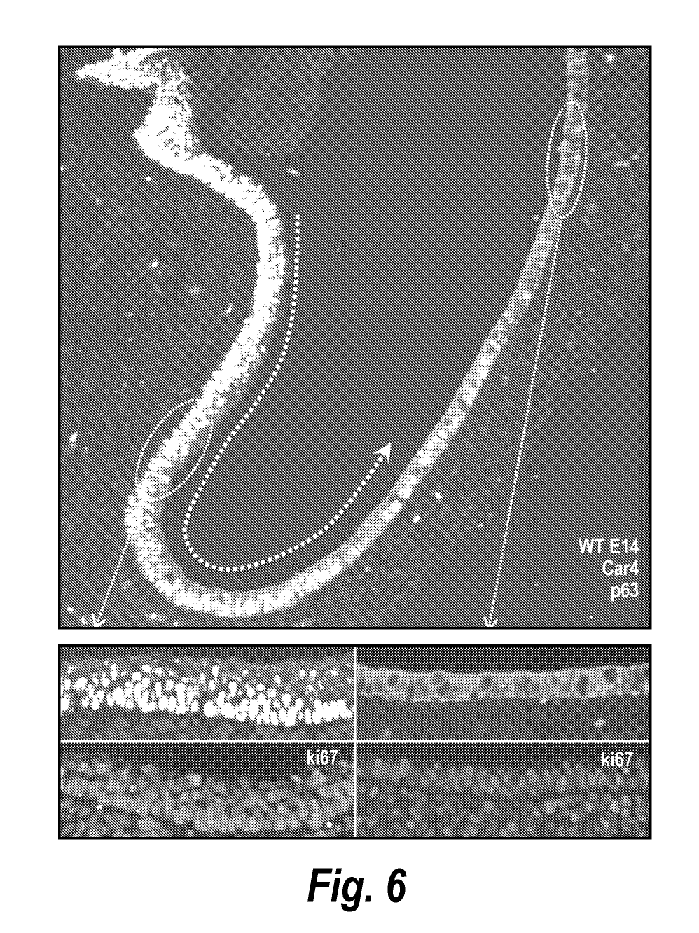

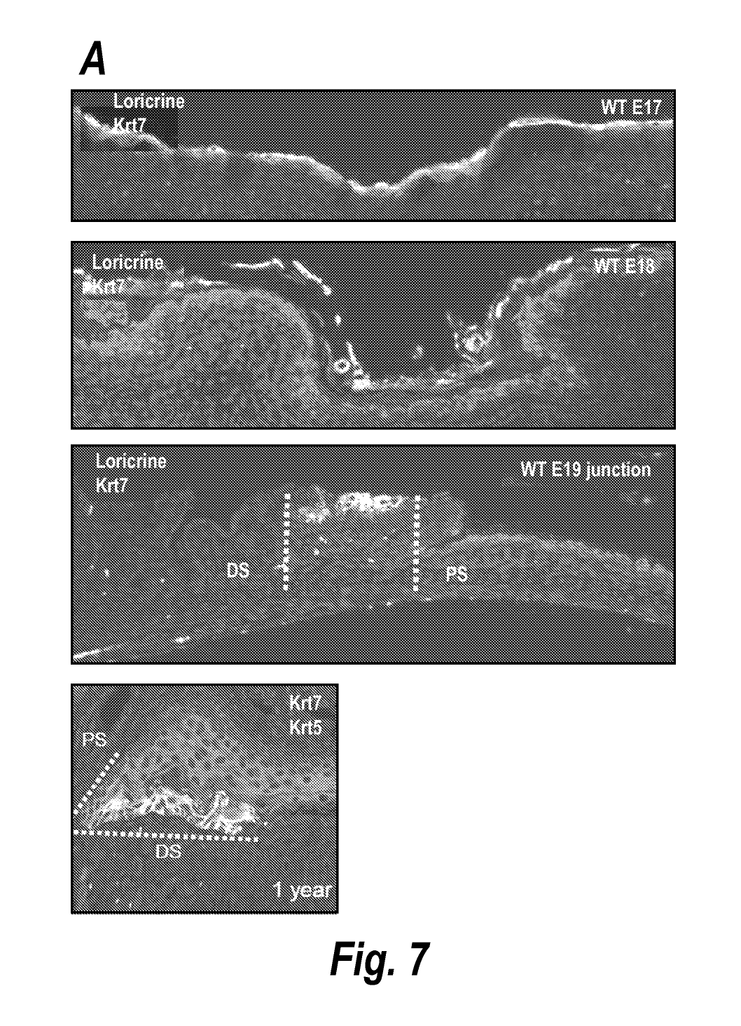

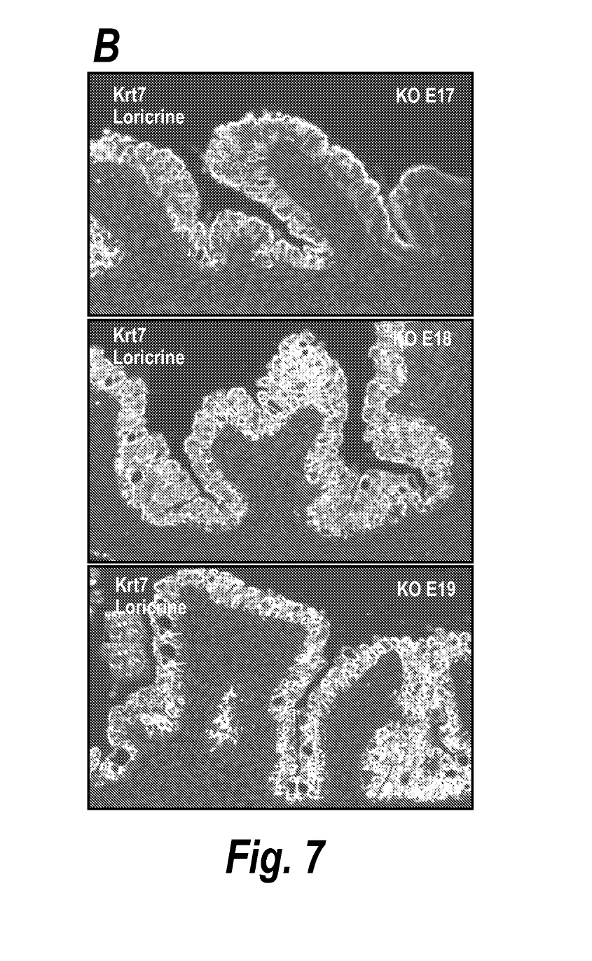

To identify the source of the metaplasia evident in the p63 null proximal stomach, known biomarkers of Barrett's metaplasia were used to perform a retrospective analysis of embryological development. Using antibodies to claudin 3 (Cdn3), keratin 7 (Krt7), and carbonic anhydrase 4 (Car4) that show robust staining of E18 metaplasia (FIG. 3a), it was demonstrated that metaplasia was present as early as E14, when the metaplastic tissue in the stomach presents as a highly proliferative columnar epithelium marked by Car4, Cdn3, and Ki67 expression (FIG. 3b). One day earlier in development, at E13, Car-4-positive cells were detected in a single layer in the stomach of the mutant embryos on an extended region of basement membrane of the proximal stomach (FIG. 3c). Significantly, the wild type E13 embryos also showed a similar population of Car-4-positive cells at the basement membrane of the proximal stomach (FIG. 3c), demonstrating that this cell population is the origin of the observed metaplasia in the mutants, and that at E13, the evolution of the metaplasia had not been initiated. Given the both the p63 null and the wild type embryos displayed an apparently similar layer of Car-4-positive cells on the basement membrane of the proximal stomach at E13, it was unclear why the p63 null embryos went on to develop a Barrett's-like metaplasia while the wild type embryos did not. p63 is a transcription factor required for long-term self-renewal of stem cells of stratified epithelia but is not required for their commitment to stem cells nor for their differentiation (Yang et al., 1999; Senoo et al., 2007 supra). Strong p63 expression was first detected at E13 in a population of cells at the esophageal gastric junction and this expression is notably weaker in cells that extended distally to the junction of Car4 cells (FIG. 3d). By E14, this population of p63-positive cells appears to extend to and actually among and under the population of Car4/Cdn3 positive cells in an anterior-posterior gradient (FIG. 3d), such that many of the Car4/Cdn3 cells are displaced from the basement membrane to an apical position about the p63-expressing cells. Remarkably, whereas the Car4 expressing cells positioned on the basement membrane at the posterior end of this gradient are highly proliferative, those undermined by p63-expressing cells show significantly reduced cell cycle activity as judged by decreased Ki67 expression (FIG. 6). In the p63 null embryo, the Car4 cells are not undermined by epithelial cells at E14 and instead appear to rapidly propagate to a columnar epithelium. This lack of epithelial cells is due to the absence of p63 and their loss of self-renewal capacity, as has been demonstrated for stem cells of other squamous epithelia including the epidermis and thymic epithelial cells (Senoo et al., 2007 supra). It was also noted that both the epidermal and thymic epithelial stem cells still undergo complete differentiation programs in the absence of p63, no evidence of squamous differentiation at any stage of the metaplasia was found in the p63 null embryos (FIG. 7). These data demonstrate that the Car4 cells that nucleate the metaplasia in the p63 null embryos lack inherent squamous differentiation programs.

### Example 4

### Undermined Embryonic Epithelium is Retained at the Squamocolumnar Junction in Adult Mammals

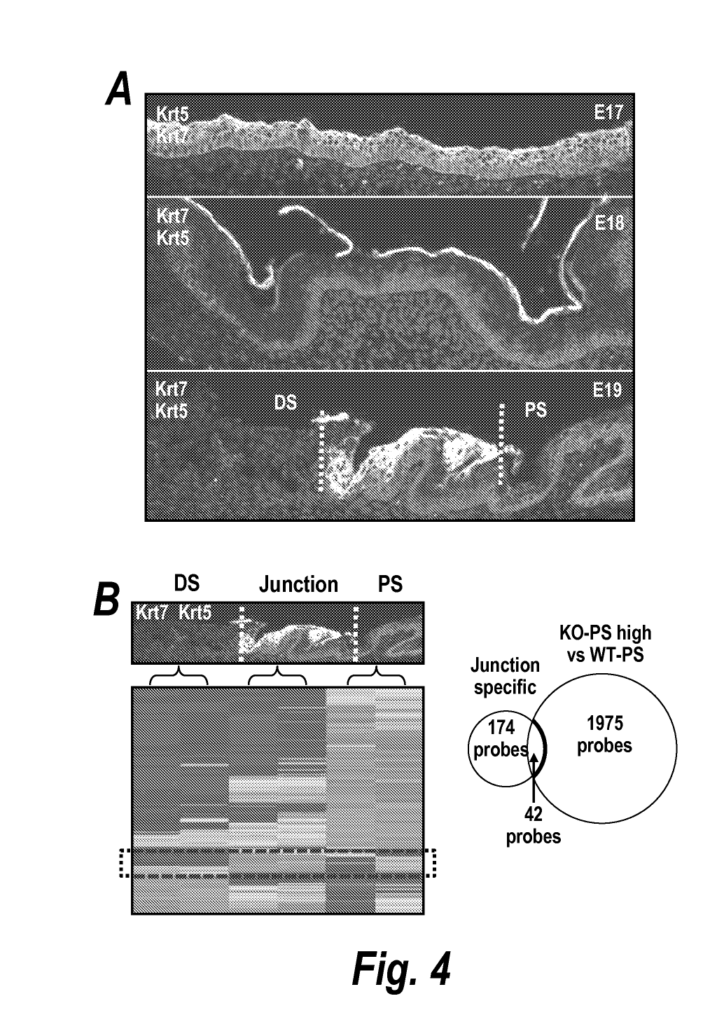

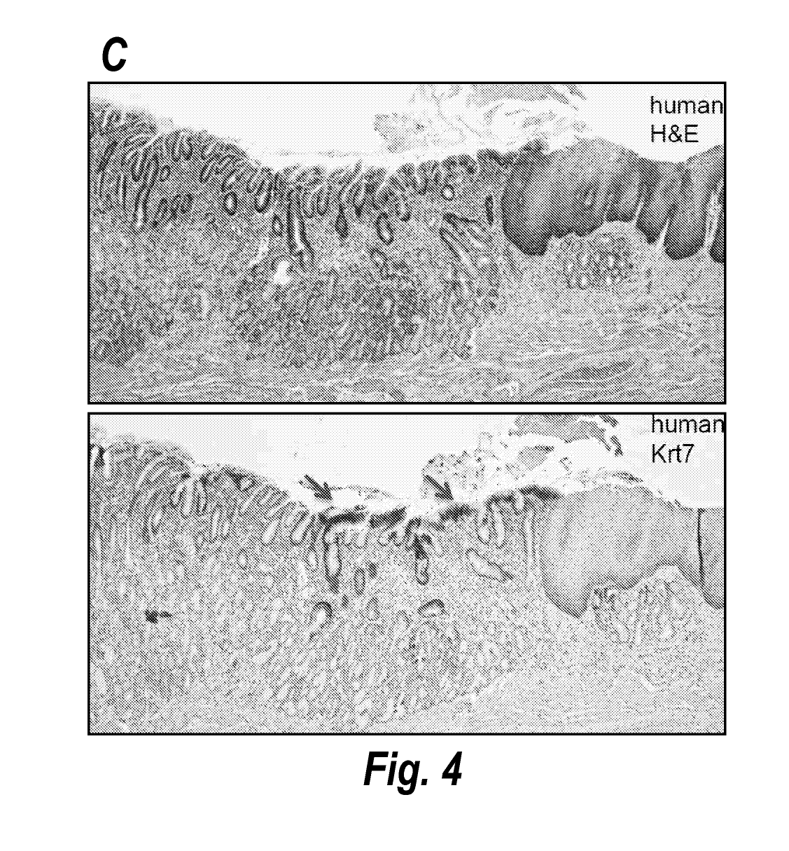

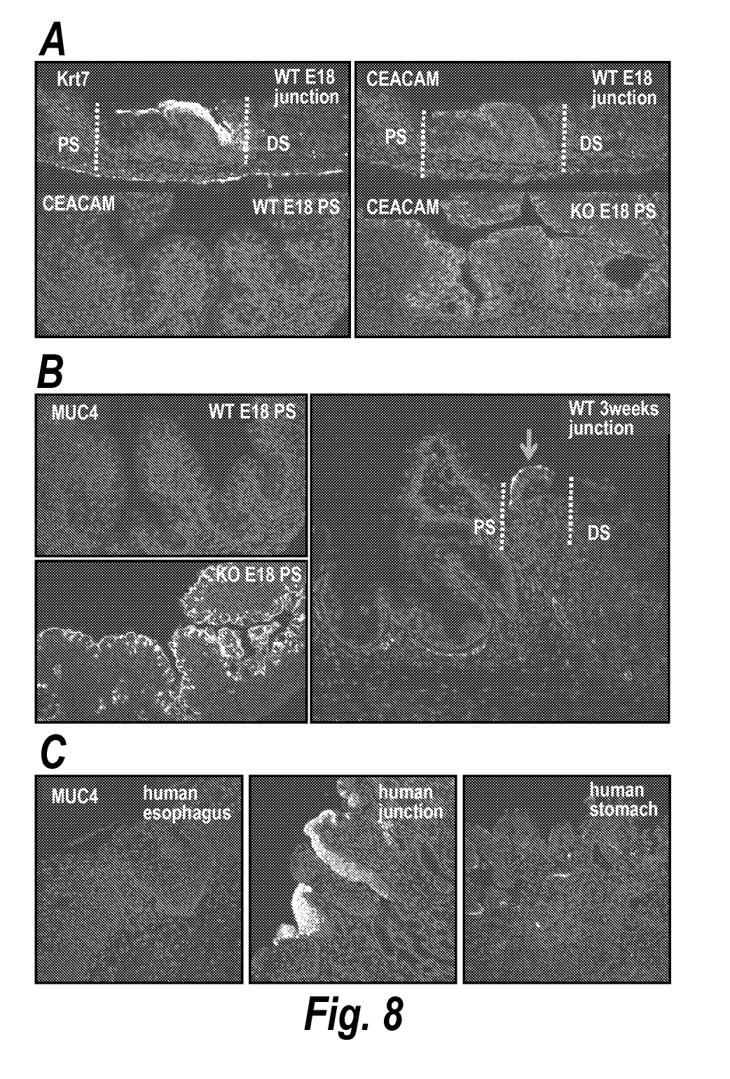

To determine the ultimate fate of the Car4/Cdn3-expressing cells undermined by the p63-positive cells at E14, their fate was followed from E14 through to adulthood in wild type mice. By E15, these cells cease expression of Car4 but retain Cdn3 expression and assume expression of keratin 7 (not shown). At E17, these cells maintain their apical position above the stratifying squamous epithelia in the proximal stomach (FIG. 4a), but at E18 undergo a wholesale detachment from the underlying epithelia in large sheets (FIG. 4b). By E19, the Krt7-expressing cells have exfoliated from the entire proximal stomach with the exception of a discrete population of cells (numbering approximately 30 cells in cross-section) remaining precisely at the squamocolumnar junction (FIG. 4c). A similar population of Krt7-positive cells was observed in mice at three weeks of age (FIG. 4d) and as late as one year (not shown). Transcriptome analysis of RNA derived by microdissection of the squamocolumnar junction and compared with adjacent squamous and columnar regions of the three-week-old mouse stomach revealed a distinct junctional signature marked by carcinoembryonic antigen (CEACAM1), Muc4, and Gabrp, all of which were significantly elevated (11-40×) in metaplasia from E18 p63 knockout embryos (FIG. 8). The similarity between the persistent embryonic cells at this junction in wild type mice and the embryonic metaplasia in the p63 null embryos are further links to their common origins in the Car-4-positive cells observed at E13. These data were directly supported by laser capture microdissection (LCM) of the junction Krt7-positive cells from three-week only mice compared with epithelial regions of the proximal and distal stomach (FIG. 4e). Lastly, it was determined if gastroesophageal junction tissues obtained from autopsies of humans without overt Barrett's also possessed cells similar to those described in mice. Antibodies to keratin 7, CEACAM1, and mucin 4 all revealed a discrete population of positive cells at the human gastroesophageal junction (FIG. 4e). These data demonstrate that the retention of embryonic epithelia at the squamocolumnar junction in the gastrointestinal tract is a common feature of adult mammals.

### Example 5

### Retained Embryonic Epithelia Nucleate Barrett's-Like Metaplasia

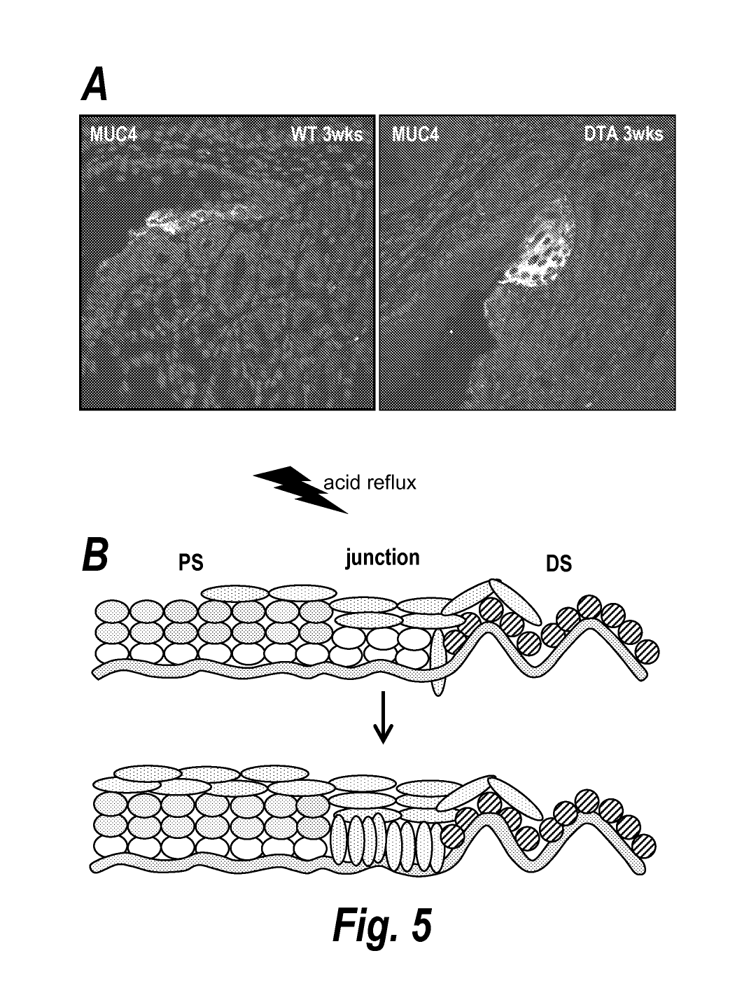

The persistence of a discrete population of cells having a lineage relation to an embryonic version of Barrett's metaplasia raised the possibility that they might spawn similar metaplasias in the adult. To test this hypothesis, mice were generated in which diptheria toxin A was conditionally expressed in basal cells of stratified epithelia by crossing the ROSA26-tm-DTA mouse (see Ivanova et al. 2005 supra) with one having a Tamoxifen-dependent Cre recombinase under the control of the Krt14 promoter Vasioukhin et al. (hereafter the DTA-Krt14Cre mouse). Treatment of three-week-old DTA-Krt14Cre mice with Tamoxifen resulted in a rapid expansion of the Krt7-expressing cells from their original site at the squamocolumnar junction to more anterior regions of the proximal stomach (FIG. 5a). Significantly, accompanying the expansion of these Krt7-expressing cells was their intimate association with the basement membrane that was presumably vacated by basal cells weakened or killed as a consequence of Cre-mediated diptheria toxin A expression (FIG. 5b). In accord with their rapid expansion, these cells also show high levels of Ki67 indicative of cell cycle progression (FIG. 5c). Overall, the progression of this Barrett's-like metaplasia in the DTA-Krt14Cre mouse underscores the need of these retained embryonic cells to access the basement membrane for expansion, in turn, made possible by damage to the resident squamous epithelia. Taken together these data demonstrate that retained embryonic epithelia nucleate Barrett's-like metaplasia.

### Example 6

### Gene Expression of Barrett's Esophagus Progenitor Cell Compared to Squamous and Gastric Cardia Progenitor Cells

Expression microarrays were used to compare the mRNA expression of an isolated clonal population of Barrett's esophagus progenitor cells and a clonal population of squamous progenitor cells. The results of this comparison are shown in Table ZZ, below.

TABLE ZZ

| | | | p-value | | Fold-Change |
|---|---|---|---|---|---|
| Gene | | p-value | (Barrett's vs. | Ratio (Barrett's | (Barrett's vs. |
| Symbol | RefSeq No. | (Attribute) | Squamous) | vs. Squamous) | Squamous) |
| FABP1 | NM_001443 | 2.34E−09 | 0.085908 | 1.47483 | 1.47483 |
| CPS1 | NM_001122633 | 2.60E−07 | 0.010034 | 0.434438 | −2.30182 |
| FABP2 | NM_000134 | 5.39E−08 | 0.386115 | 1.19002 | 1.19002 |
| PRSS2 | NM_002770 | 2.27E−05 | 0.566736 | 0.808094 | −1.23748 |
| KRT20 | NM_019010 | 5.33E−07 | 0.149126 | 1.44708 | 1.44708 |
| DMBT1 | NM_007329 | 7.79E−09 | 0.774347 | 1.03935 | 1.03935 |
| SI | NM_001041 | 8.23E−09 | 0.022277 | 1.43479 | 1.43479 |
| MTTP | NM_000253 | 2.97E−09 | 0.003091 | 1.54794 | 1.54794 |
| RBP2 | NM_004164 | 2.08E−07 | 0.080011 | 1.42961 | 1.42961 |
| MT1H | NM_005951 | 2.21E−06 | 0.833756 | 0.952728 | −1.04962 |
| CLCA1 | NM_001285 | 1.94E−07 | 0.284001 | 1.20283 | 1.20283 |
| KGFLP2 | NR_003670 | 7.43E−06 | 0.685646 | 0.906698 | −1.1029 |
| GUCY2C | NM_004963 | 3.89E−09 | 0.002247 | 1.56122 | 1.56122 |
| GSTA2 | NM_000846 | 0.000164 | 0.018224 | 4.07423 | 4.07423 |
| CDH17 | NM_004063 | 3.78E−09 | 5.02E−07 | 9.55456 | 9.55456 |
| C17orf78 | NM_173625 | 0.00023 | 0.780594 | 0.905833 | −1.10396 |
| GPR128 | NM_032787 | 3.01E−08 | 0.009887 | 1.4728 | 1.4728 |
| TM4SF4 | NM_004617 | 1.49E−08 | 2.71E−07 | 14.0435 | 14.0435 |
| GJA1 | NM_000165 | 0.000666 | 0.000192 | 0.04303 | −23.2396 |
| OTC | NM_000531 | 1.40E−07 | 0.000185 | 2.93928 | 2.93928 |
| BEX1 | NM_018476 | 3.43E−05 | 0.947974 | 0.983379 | −1.0169 |
| HIST1H1A | NM_005325 | 1.95E−07 | 0.00842 | 1.65384 | 1.65384 |
| OLFM4 | NM_006418 | 1.75E−10 | 4.36E−09 | 15.251 | 15.251 |
| LOC29034 | NR_002763 | 1.07E−07 | 0.167948 | 1.18132 | 1.18132 |
| BTNL3 | NM_197975 | 4.86E−06 | 0.027782 | 1.68628 | 1.68628 |
| DPY19L2P2 | NR_003561 | 0.000999 | 0.228149 | 1.64931 | 1.64931 |
| CPE | NM_001873 | 1.65E−06 | 0.001361 | 0.513199 | −1.94856 |
| RGS5 | NM_003617 | 1.02E−05 | 0.025869 | 1.76086 | 1.76086 |
| CPVL | NM_019029 | 1.05E−06 | 0.005975 | 0.643632 | −1.55368 |
| DSG3 | NM_001944 | 7.14E−10 | 2.84E−10 | 0.01217 | −82.1708 |
| TM4SF20 | NM_024795 | 3.07E−07 | 0.20159 | 1.17546 | 1.17546 |
| SLC38A11 | NM_173512 | 2.05E−06 | 0.431078 | 1.12989 | 1.12989 |
| ADH4 | NM_000670 | 2.50E−07 | 0.005823 | 1.58646 | 1.58646 |
| CEACAM6 | NM_002483 | 3.08E−05 | 0.001177 | 8.35385 | 8.35385 |
| SYNPR | NM_001130003 | 2.23E−05 | 0.348375 | 1.22134 | 1.22134 |
| ALDOB | NM_000035 | 2.00E−07 | 2.93E−05 | 3.90662 | 3.90662 |
| FAM13A | NM_001015045 | 2.05E−05 | 0.471827 | 1.15247 | 1.15247 |
| SLC17A4 | NM_005495 | 4.81E−06 | 0.237349 | 1.23116 | 1.23116 |
| CACNA2D1 | NM_000722 | 9.75E−08 | 0.00453 | 1.50218 | 1.50218 |
| ATF7IP2 | NM_024997 | 2.12E−05 | 0.759455 | 0.946229 | −1.05683 |
| MEP1A | NM_005588 | 1.31E−06 | 0.075815 | 1.32331 | 1.32331 |
| RBM46 | NM_144979 | 7.01E−05 | 0.19628 | 0.748349 | −1.33628 |
| ZG16 | NM_152338 | 8.01E−05 | 0.648568 | 1.11182 | 1.11182 |
| REG4 | NM_001159352 | 2.83E−08 | 2.50E−07 | 11.2549 | 11.2549 |
| MUC17 | NM_001040105 | 1.67E−06 | 8.78E−05 | 5.67432 | 5.67432 |
| LGR5 | NM_003667 | 3.55E−07 | 0.000603 | 2.01122 | 2.01122 |
| PRSS1 | NM_002769 | 8.44E−05 | 0.161547 | 1.42939 | 1.42939 |
| SLC2A2 | NM_000340 | 3.42E−06 | 0.079066 | 1.35541 | 1.35541 |
| PHYHIPL | NM_032439 | 1.38E−05 | 0.065554 | 1.46452 | 1.46452 |
| ACE2 | NM_021804 | 1.43E−07 | 0.000437 | 1.8846 | 1.8846 |
| CCND2 | NM_001759 | 5.19E−05 | 2.30E−05 | 0.091416 | −10.939 |
| SULT1E1 | NM_005420 | 3.45E−07 | 1.97E−07 | 0.063952 | −15.6367 |
| SLC5A1 | NM_000343 | 8.44E−06 | 0.001409 | 2.71989 | 2.71989 |
| SEMA6A | NM_020796 | 7.77E−07 | 0.00025 | 2.72895 | 2.72895 |
| MT1L | NR_001447 | 0.004952 | 0.000668 | 0.044037 | −22.7081 |
| HMGCS2 | NM_005518 | 3.07E−07 | 1.87E−05 | 3.67728 | 3.67728 |
| MGAT4A | NM_012214 | 6.97E−06 | 0.000308 | 3.50807 | 3.50807 |
| UGT2B17 | NM_001077 | 5.68E−06 | 0.001365 | 2.32882 | 2.32882 |
| C15orf48 | NM_032413 | 1.19E−08 | 5.90E−06 | 2.33056 | 2.33056 |
| CISD2 | NM_001008388 | 0.000711 | 0.031534 | 0.574783 | −1.73979 |
| SST | NM_001048 | 0.000975 | 0.565834 | 1.17549 | 1.17549 |
| SPC25 | NM_020675 | 0.033633 | 0.373577 | 0.690568 | −1.44808 |
| PLA2G12B | NM_032562 | 1.29E−05 | 0.415062 | 1.12817 | 1.12817 |
| LGALS2 | NM_006498 | 1.72E−08 | 3.07E−06 | 2.57988 | 2.57988 |
| NR1H4 | NM_005123 | 5.91E−06 | 0.0257 | 1.45199 | 1.45199 |
| UGT3A1 | NM_152404 | 1.50E−05 | 0.184394 | 1.22895 | 1.22895 |
| GIP | NM_004123 | 0.066104 | 0.751954 | 1.19265 | 1.19265 |
| LOC147727 | NR_024333 | 1.58E−05 | 0.188258 | 0.831438 | −1.20274 |
| ABCG2 | NM_004827 | 0.000813 | 0.013104 | 0.524561 | −1.90636 |
| OCR1 | AF314543 | 0.024574 | 0.808636 | 1.12556 | 1.12556 |
| LMBR1 | NM_022458 | 0.008559 | 0.00191 | 0.095088 | −10.5166 |
| A1CF | NM_138933 | 7.40E−07 | 4.35E−05 | 3.6505 | 3.6505 |
| IGF2BP1 | NM_006546 | 1.50E−07 | 0.001471 | 1.46936 | 1.46936 |
| TSPAN7 | NM_004615 | 0.000601 | 0.978363 | 1.00601 | 1.00601 |
| CEACAM7 | NM_006890 | 3.74E−06 | 0.064122 | 1.28262 | 1.28262 |
| MYB | NM_001130173 | 4.65E−06 | 0.000341 | 2.52787 | 2.52787 |
| CFI | NM_000204 | 8.87E−06 | 0.000331 | 3.21941 | 3.21941 |
| SLC10A2 | NM_000452 | 6.69E−05 | 0.141134 | 1.31867 | 1.31867 |
| UGT2A3 | NR_024010 | 1.48E−07 | 2.25E−06 | 7.27273 | 7.27273 |
| IFITM1 | NM_003641 | 6.18E−05 | 6.04E−05 | 0.139414 | −7.17288 |
| TMEM20 | NM_001134658 | 0.000102 | 0.498743 | 1.12714 | 1.12714 |
| TNFRSF11B | NM_002546 | 1.34E−05 | 0.000327 | 3.09331 | 3.09331 |
| SMOC2 | NM_022138 | 8.35E−05 | 0.047803 | 1.51645 | 1.51645 |
| TGFBI | NM_000358 | 0.000306 | 8.74E−05 | 0.107698 | −9.28521 |
| GPA33 | NM_005814 | 0.00014 | 0.144044 | 1.33829 | 1.33829 |
| NELL2 | NM_001145108 | 4.57E−05 | 0.158833 | 1.26394 | 1.26394 |
| ATP1B3 | NM_001679 | 7.11E−07 | 1.23E−07 | 0.100257 | −9.97437 |
| FGF9 | NM_002010 | 2.98E−05 | 0.295931 | 1.16051 | 1.16051 |
| FOLH1 | NM_004476 | 1.41E−05 | 0.014508 | 1.5141 | 1.5141 |
| RGS2 | NM_002923 | 7.55E−06 | 0.460428 | 0.886527 | −1.128 |
| NAT2 | NM_000015 | 4.42E−05 | 0.003952 | 2.07047 | 2.07047 |
| CCL25 | NM_005624 | 8.63E−05 | 0.142099 | 1.28954 | 1.28954 |
| SEMA6D | NM_153618 | 1.55E−05 | 0.147042 | 0.838921 | −1.19201 |
| ANXA13 | NM_001003954 | 2.27E−08 | 1.51E−07 | 15.5115 | 15.5115 |
| KLHL23 | ENST00000392647 | 8.54E−06 | 0.00107 | 2.12725 | 2.12725 |
| GSTA1 | NM_145740 | 1.25E−06 | 3.65E−06 | 13.4613 | 13.4613 |
| S100G | NM_004057 | 6.67E−05 | 0.082683 | 1.37934 | 1.37934 |
| LCT | NM_002299 | 1.06E−05 | 0.047808 | 1.31511 | 1.31511 |
| FAM5C | NM_199051 | 4.88E−06 | 0.405939 | 1.08729 | 1.08729 |
| ANPEP | NM_001150 | 3.32E−06 | 0.005603 | 1.68274 | 1.68274 |
| HIST1H2AE | NM_021052 | 0.001216 | 0.2424 | 1.31829 | 1.31829 |
| SLC11A2 | NM_000617 | 1.91E−06 | 0.002027 | 1.56795 | 1.56795 |
| LRRC19 | NM_022901 | 4.02E−06 | 0.001068 | 1.87406 | 1.87406 |
| SLC27A2 | NM_003645 | 1.60E−05 | 0.000193 | 3.33023 | 3.33023 |
| LDHC | NM_002301 | 5.51E−06 | 0.128855 | 1.17817 | 1.17817 |
| SCGN | NM_006998 | 0.000129 | 0.105465 | 1.31481 | 1.31481 |
| GPR160 | NM_014373 | 2.16E−05 | 0.000152 | 3.88993 | 3.88993 |
| SLC16A10 | NM_018593 | 0.000465 | 0.188271 | 1.30369 | 1.30369 |
| CLRN3 | NM_152311 | 4.69E−08 | 2.24E−07 | 11.4491 | 11.4491 |
| C12orf28 | BC143553 | 1.27E−05 | 0.269447 | 1.18243 | 1.18243 |
| SATB1 | NM_002971 | 0.000101 | 6.00E−05 | 0.132514 | −7.54637 |
| GOLT1A | NM_198447 | 4.68E−07 | 5.93E−05 | 1.92107 | 1.92107 |
| UFM1 | NM_016617 | 1.64E−05 | 0.140695 | 0.862813 | −1.159 |
| HIBCH | NM_014362 | 0.011898 | 0.480496 | 0.835616 | −1.19672 |
| L1TD1 | NM_019079 | 0.000304 | 0.000105 | 0.145075 | −6.89301 |
| HOXA9 | NM_152739 | 2.96E−05 | 1.09E−05 | 0.090174 | −11.0897 |
| TPH1 | NM_004179 | 0.000951 | 0.822371 | 1.04004 | 1.04004 |
| HEPH | NM_138737 | 7.75E−08 | 1.80E−06 | 3.68292 | 3.68292 |
| BMS1P5 | NR_003611 | 0.240068 | 0.968687 | 1.02442 | 1.02442 |
| ASAH2 | NM_019893 | 7.62E−05 | 0.145436 | 1.23563 | 1.23563 |
| KIAA1324 | NM_020775 | 3.22E−08 | 0.00884 | 1.48311 | 1.48311 |
| ALDOC | NM_005165 | 2.49E−06 | 0.330685 | 1.10185 | 1.10185 |
| KPNA2 | NM_002266 | 0.022754 | 0.772384 | 1.09878 | 1.09878 |
| NEUROD1 | NM_002500 | 0.06316 | 0.666654 | 1.17268 | 1.17268 |
| MS4A8B | NM_031457 | 5.65E−06 | 0.003764 | 1.48823 | 1.48823 |
| EPHB2 | NM_017449 | 0.001129 | 0.180313 | 0.805308 | −1.24176 |
| MSI1 | NM_002442 | 9.22E−06 | 0.012047 | 1.38697 | 1.38697 |
| IFNK | NM_020124 | 0.002165 | 0.001768 | 0.168645 | −5.92962 |
| FGFBP1 | NM_005130 | 1.79E−08 | 2.67E−09 | 0.032053 | −31.1981 |
| CDKN1B | NM_004064 | 3.54E−05 | 0.900479 | 0.985955 | −1.01425 |
| TFPI | NM_006287 | 1.26E−05 | 2.84E−05 | 7.05765 | 7.05765 |
| STAMBPL1 | NM_020799 | 4.70E−06 | 0.109009 | 0.878368 | −1.13848 |
| NLGN4Y | NM_014893 | 4.39E−05 | 2.77E−05 | 0.185298 | −5.39672 |
| PLD1 | NM_002662 | 0.000446 | 0.701913 | 0.934448 | −1.07015 |
| APOBEC3B | NM_004900 | 0.001419 | 0.321714 | 1.22865 | 1.22865 |
| MEP1B | NM_005925 | 5.41E−05 | 0.120667 | 1.21259 | 1.21259 |
| | — | 0.001183 | 0.000292 | 0.157535 | −6.34781 |
| EPHX2 | NM_001979 | 1.10E−06 | 0.122339 | 0.898451 | −1.11303 |
| XRCC4 | NM_022550 | 0.001579 | 0.024582 | 2.32654 | 2.32654 |
| GAS2 | NM_005256 | 3.49E−05 | 0.022857 | 1.37523 | 1.37523 |
| DPP10 | NM_020868 | 0.000864 | 0.293544 | 1.1938 | 1.1938 |
| TLR4 | NR_024168 | 9.63E−05 | 0.007119 | 1.66233 | 1.66233 |
| LSAMP | NM_002338 | 2.16E−05 | 0.002367 | 1.65097 | 1.65097 |
| SEPT7 | NM_001788 | 0.01691 | 0.003851 | 0.18092 | −5.52729 |
| CCNB2 | NM_004701 | 0.009939 | 0.814129 | 0.952239 | −1.05016 |
| MT1A | NM_005946 | 1.80E−05 | 0.000411 | 0.439688 | −2.27434 |
| C2orf43 | BC017473 | 0.002035 | 0.506445 | 1.12716 | 1.12716 |
| EML4 | NM_019063 | 0.003235 | 0.085036 | 1.55247 | 1.55247 |
| CKS2 | NM_001827 | 2.48E−05 | 0.183434 | 1.13856 | 1.13856 |
| CYP2B6 | NM_000767 | 0.000209 | 0.002722 | 2.38855 | 2.38855 |
| CCDC34 | NM_030771 | 4.73E−05 | 0.48388 | 0.934202 | −1.07043 |
| ADH6 | NM_001102470 | 2.18E−06 | 3.67E−05 | 2.77804 | 2.77804 |
| ATP8A1 | NM_006095 | 9.35E−06 | 2.53E−05 | 5.38379 | 5.38379 |
| FAR2 | NM_018099 | 3.78E−07 | 0.066621 | 1.16312 | 1.16312 |
| TF | NM_001063 | 7.43E−06 | 0.63424 | 1.03621 | 1.03621 |
| MYO1B | NM_001130158 | 1.47E−06 | 9.52E−07 | 0.114773 | −8.71287 |
| SLC35D1 | NM_015139 | 0.066551 | 0.909823 | 0.965605 | −1.03562 |
| CXorf52 | AY168775 | 0.026084 | 0.012721 | 0.219586 | −4.55402 |
| PCDH11Y | NM_032971 | 0.368856 | 0.665542 | 1.29083 | 1.29083 |
| SERPINE2 | NM_001136529 | 2.73E−07 | 9.54E−08 | 0.032271 | −30.9872 |
| ERP27 | NM_152321 | 0.002033 | 0.712918 | 1.07767 | 1.07767 |
| DNAJC2 | NM_014377 | 0.000601 | 0.000118 | 0.173456 | −5.76516 |
| PCDH20 | NM_022843 | 0.000951 | 0.136199 | 1.2937 | 1.2937 |
| HNF4G | NM_004133 | 3.36E−07 | 5.08E−07 | 11.2611 | 11.2611 |
| HIST1H3G | NM_003534 | 7.92E−05 | 0.010087 | 1.48882 | 1.48882 |
| HPDL | NM_032756 | 0.001394 | 0.024925 | 1.72923 | 1.72923 |
| SH3PXD2A | NM_014631 | 2.02E−05 | 3.22E−06 | 0.052282 | −19.1269 |
| COX18 | NM_173827 | 0.001081 | 0.111706 | 1.32598 | 1.32598 |
| HHLA2 | NM_007072 | 1.26E−05 | 2.55E−05 | 5.74993 | 5.74993 |
| ZNF770 | NM_014106 | 2.22E−05 | 5.56E−06 | 0.155206 | −6.44304 |
| LYPLA1 | NM_006330 | 5.75E−05 | 1.43E−05 | 0.112802 | −8.86512 |
| DHRS11 | NM_024308 | 0.000217 | 0.016866 | 1.61863 | 1.61863 |
| EPB41L2 | NM_001431 | 0.003371 | 0.07753 | 1.57053 | 1.57053 |
| EXOC3 | AK074086 | 1.49E−06 | 0.003021 | 1.31235 | 1.31235 |
| GHRL | NR_024138 | 0.027865 | 0.649993 | 1.11777 | 1.11777 |
| DACH1 | NM_080759 | 0.000217 | 0.135116 | 1.21114 | 1.21114 |
| SPARC | NM_003118 | 1.66E−06 | 6.35E−07 | 0.131619 | −7.5977 |
| SLCO4C1 | NM_180991 | 3.06E−05 | 0.010122 | 1.40569 | 1.40569 |
| KLHL23 | NM_144711 | 0.000249 | 0.00203 | 2.3695 | 2.3695 |
| KRT6B | NM_005555 | 9.83E−11 | 3.36E−11 | 0.019068 | −52.4439 |
| EPCAM | NM_002354 | 1.17E−07 | 1.65E−07 | 10.5781 | 10.5781 |
| IL20RB | NM_144717 | 7.88E−07 | 2.82E−07 | 0.024169 | −41.3761 |
| MEIS2 | NM_172316 | 5.41E−06 | 0.00904 | 1.34573 | 1.34573 |
| MMP12 | NM_002426 | 0.003373 | 0.379449 | 1.17563 | 1.17563 |
| ACPL2 | NM_152282 | 8.11E−06 | 0.006848 | 1.32703 | 1.32703 |
| TIMP3 | NM_000362 | 3.21E−07 | 9.08E−08 | 0.107177 | −9.33032 |
| CXCL14 | NM_004887 | 0.000211 | 0.000136 | 0.225227 | −4.43996 |
| METTL6 | NM_152396 | 0.001275 | 0.000276 | 0.240784 | −4.15311 |
| ZNF770 | NM_014106 | 1.21E−06 | 4.25E−07 | 0.259739 | −3.85002 |
| CLDND1 | NM_001040199 | 0.000346 | 6.39E−05 | 0.254923 | −3.92275 |
| RAET1L | NM_130900 | 5.71E−06 | 1.04E−06 | 0.040894 | −24.4532 |
| SDAD1 | NM_018115 | 0.022444 | 0.003522 | 0.263153 | −3.80007 |
| PLEKHF2 | NM_024613 | 0.005965 | 0.001481 | 0.20412 | −4.89908 |
| TMEM117 | NM_032256 | 0.000172 | 3.16E−05 | 0.205018 | −4.87762 |
| RASA1 | NM_002890 | 0.000185 | 5.26E−05 | 0.246229 | −4.06126 |
| S100A16 | NM_080388 | 2.27E−05 | 4.60E−06 | 0.177264 | −5.64132 |
| KCTD9 | NM_017634 | 0.000344 | 5.35E−05 | 0.225387 | −4.4368 |
| GRHL1 | NM_014552 | 2.68E−07 | 1.05E−07 | 0.12599 | −7.93715 |
| ARHGAP29 | NM_004815 | 8.76E−05 | 3.38E−05 | 0.137068 | −7.29567 |
| BNIP2 | NM_004330 | 4.25E−05 | 2.47E−05 | 0.19726 | −5.06945 |
| MARCH7 | NM_022826 | 0.017224 | 0.00286 | 0.282151 | −3.5442 |
| RAB23 | NM_016277 | 0.001104 | 0.000412 | 0.293115 | −3.41163 |
| STK17A | NM_004760 | 0.001954 | 0.000537 | 0.142798 | −7.00291 |
| REEP3 | ENST00000298249 | 0.000142 | 4.20E−05 | 0.194872 | −5.13157 |
| ATL2 | NM_022374 | 0.002578 | 0.000458 | 0.253567 | −3.94373 |
| MALT1 | NM_006785 | 3.81E−07 | 8.68E−08 | 0.216331 | −4.62254 |
| LOC554203 | NR_024582 | 0.005588 | 0.002116 | 0.264831 | −3.776 |
| DUSP11 | NM_003584 | 8.27E−05 | 2.00E−05 | 0.230212 | −4.34382 |
| IGF2BP2 | NM_006548 | 0.00154 | 0.001246 | 0.314178 | −3.18291 |
| SEPT10 | NM_144710 | 0.005078 | 0.000948 | 0.278222 | −3.59426 |
| REPS1 | NM_031922 | 0.001111 | 0.000185 | 0.275492 | −3.62987 |
| C3orf14 | AF236158 | 0.000139 | 0.000224 | 0.201203 | −4.9701 |
| ADK | NM_006721 | 6.58E−05 | 2.37E−05 | 0.293314 | −3.40932 |
| SSR3 | NM_007107 | 0.010975 | 0.001855 | 0.262664 | −3.80714 |
| PRRG4 | NM_024081 | 3.02E−05 | 5.11E−06 | 0.279794 | −3.57406 |
| PDPN | NM_006474 | 8.09E−07 | 3.12E−07 | 0.113888 | −8.78052 |
| KIAA1586 | NM_020931 | 1.65E−05 | 6.29E−06 | 0.20561 | −4.86357 |
| PEX3 | NM_003630 | 1.98E−05 | 7.68E−06 | 0.169278 | −5.90744 |
| | — | 0.000761 | 0.000453 | 0.290274 | −3.44502 |
| EIF2AK2 | NM_002759 | 0.012021 | 0.00241 | 0.265477 | −3.76681 |
| GTF2F2 | NM_004128 | 0.000579 | 0.000127 | 0.319385 | −3.13102 |
| SMYD2 | NM_020197 | 7.87E−05 | 1.92E−05 | 0.332855 | −3.00431 |
| CTSC | NM_001814 | 1.37E−07 | 4.24E−08 | 0.142682 | −7.0086 |
| MPP7 | NM_173496 | 1.95E−07 | 5.74E−07 | 0.291796 | −3.42705 |
| GDAP1 | NM_018972 | 1.52E−06 | 2.78E−06 | 0.317421 | −3.15039 |
| FN1 | NM_212482 | 0.000112 | 0.0001 | 0.165688 | −6.03545 |
| TROVE2 | NM_004600 | 0.004196 | 0.001181 | 0.324991 | −3.07701 |
| C1orf149 | NM_022756 | 0.000508 | 0.000104 | 0.338246 | −2.95643 |
| CLEC2B | NM_005127 | 0.003209 | 0.001006 | 0.197704 | −5.05808 |
| ALS2CR4 | NM_001044385 | 1.40E−06 | 7.40E−07 | 0.270404 | −3.69817 |
| PTPN12 | NM_002835 | 0.001268 | 0.000833 | 0.298352 | −3.35175 |
| BOD1L | NM_148894 | 0.007872 | 0.001731 | 0.31122 | −3.21316 |
| TNNT1 | NM_003283 | 3.38E−06 | 1.81E−06 | 0.202203 | −4.94552 |
| FABP7 | NM_001446 | 0.012746 | 0.00318 | 0.349501 | −2.86123 |
| HDGFRP3 | NM_016073 | 5.09E−07 | 4.68E−07 | 0.26362 | −3.79334 |
| SPRR2D | NM_006945 | 1.59E−06 | 8.94E−07 | 0.012125 | −82.4744 |
| FJX1 | NM_014344 | 4.64E−06 | 1.75E−06 | 0.175931 | −5.68403 |
| S100A14 | NM_020672 | 9.03E−05 | 2.19E−05 | 0.16304 | −6.13347 |
| MT1M | NM_176870 | 6.30E−07 | 0.004867 | 1.8299 | 1.8299 |
| LRRC37B2 | NR_015341 | 0.000454 | 0.000132 | 0.296469 | −3.37303 |
| IL18 | NM_001562 | 2.33E−06 | 1.26E−06 | 0.142113 | −7.03667 |
| GABRE | NM_004961 | 5.51E−05 | 2.97E−05 | 0.34087 | −2.93367 |
| GNPDA2 | NM_138335 | 5.96E−05 | 3.00E−05 | 0.334367 | −2.99073 |
| ELOVL4 | NM_022726 | 2.26E−07 | 9.97E−08 | 0.050865 | −19.6597 |
| WASF1 | NM_003931 | 4.90E−05 | 3.01E−05 | 0.218251 | −4.58189 |
| PIK3CA | NM_006218 | 0.000544 | 0.000205 | 0.296013 | −3.37823 |
| MBOAT2 | NM_138799 | 0.000192 | 4.28E−05 | 0.116684 | −8.57019 |
| PAR1 | AF019616 | 0.000965 | 0.000672 | 0.341623 | −2.92721 |
| IVNS1ABP | NM_006469 | 0.006048 | 0.001203 | 0.34905 | −2.86492 |
| CHIC2 | NM_012110 | 0.000122 | 2.47E−05 | 0.3046 | −3.283 |
| VSNL1 | NM_003385 | 3.02E−08 | 7.56E−08 | 0.103554 | −9.65682 |
| LRRC37A3 | NM_199340 | 0.00638 | 0.001372 | 0.365782 | −2.73387 |
| FYTTD1 | NM_001011537 | 0.004033 | 0.000927 | 0.34136 | −2.92946 |
| RNF217 | NM_152553 | 1.89E−10 | 1.33E−10 | 0.109281 | −9.1507 |
| PLA2G4A | NM_024420 | 0.006562 | 0.001299 | 0.326068 | −3.06685 |
| P2RY5 | NM_005767 | 2.90E−06 | 1.24E−06 | 0.185982 | −5.37686 |
| NT5E | NM_002526 | 2.00E−07 | 6.50E−08 | 0.078079 | −12.8076 |
| CTSL2 | NM_001333 | 1.96E−05 | 6.68E−06 | 0.138232 | −7.23424 |
| ZNF354A | NM_005649 | 0.006993 | 0.001288 | 0.36314 | −2.75376 |
| KIFAP3 | NM_014970 | 2.61E−06 | 1.91E−06 | 0.217485 | −4.59801 |
| RAB18 | NM_021252 | 4.91E−05 | 9.69E−06 | 0.26303 | −3.80185 |
| C1orf74 | BC039719 | 7.17E−05 | 2.99E−05 | 0.315305 | −3.17153 |
| RB1 | NM_000321 | 0.000478 | 8.36E−05 | 0.331091 | −3.02032 |
| CEP170 | NM_014812 | 3.10E−05 | 2.97E−05 | 0.172938 | −5.78243 |
| KIF13A | NM_022113 | 7.87E−06 | 2.98E−06 | 0.248976 | −4.01645 |
| PRKCQ | NM_006257 | 5.36E−06 | 1.90E−06 | 0.29012 | −3.44685 |
| C6orf105 | NM_001143948 | 9.93E−05 | 1.94E−05 | 0.187106 | −5.34455 |
| KRT23 | NM_015515 | 5.21E−08 | 5.19E−08 | 0.139057 | −7.19128 |
| C10orf55 | NM_001001791 | 0.004044 | 0.00097 | 0.269528 | −3.71019 |
| EFTUD1 | NM_024580 | 5.31E−05 | 1.74E−05 | 0.328301 | −3.04598 |
| EDNRA | NM_001957 | 0.00118 | 0.000393 | 0.308992 | −3.23633 |
| TMTC1 | NM_175861 | 8.69E−08 | 2.91E−08 | 0.146925 | −6.80621 |
| DUSP14 | NM_007026 | 3.77E−06 | 1.88E−06 | 0.261097 | −3.82999 |
| GPNMB | NM_001005340 | 1.01E−06 | 4.60E−07 | 0.072804 | −13.7356 |
| PRSS3 | NM_007343 | 0.001276 | 0.000294 | 0.339983 | −2.94132 |
| EMB | NM_198449 | 2.20E−07 | 1.33E−07 | 0.200409 | −4.9898 |
| SLC1A3 | NM_004172 | 3.98E−07 | 8.12E−08 | 0.19971 | −5.00727 |
| TCTEX1D2 | NM_152773 | 5.43E−08 | 2.37E−08 | 0.326036 | −3.06715 |
| NUDT11 | NM_018159 | 0.000877 | 0.000344 | 0.119 | −8.40337 |
| AIG1 | NM_016108 | 4.81E−05 | 4.03E−05 | 0.351938 | −2.84141 |
| NEDD4 | NM_006154 | 6.37E−05 | 1.52E−05 | 0.348084 | −2.87287 |
| MMP10 | NM_002425 | 0.005493 | 0.001812 | 0.110691 | −9.03418 |
| NDFIP2 | NM_019080 | 9.17E−05 | 4.06E−05 | 0.298297 | −3.35236 |
| D4S234E | NM_014392 | 2.06E−05 | 8.81E−06 | 0.140193 | −7.13301 |
| PCTK2 | NM_002595 | 8.48E−06 | 4.13E−06 | 0.310024 | −3.22556 |
| KIAA0922 | NM_001131007 | 5.88E−07 | 2.22E−07 | 0.19079 | −5.24136 |
| EFCAB2 | NR_026588 | 0.023377 | 0.006229 | 0.32682 | −3.05979 |
| RABGEF1 | NM_014504 | 0.002164 | 0.000498 | 0.369088 | −2.70938 |
| MCART1 | NR_024873 | 0.056152 | 0.013192 | 0.258038 | −3.8754 |
| IGFL3 | NM_207393 | 9.80E−08 | 2.73E−08 | 0.251981 | −3.96855 |
| ANTXR2 | NM_058172 | 5.41E−06 | 1.99E−06 | 0.242524 | −4.12331 |
| FBN2 | NM_001999 | 2.36E−07 | 1.18E−07 | 0.102509 | −9.75528 |
| SCFD1 | NM_016106 | 0.008561 | 0.00178 | 0.362447 | −2.75903 |
| C11orf60 | NM_020153 | 2.60E−06 | 6.09E−07 | 0.300207 | −3.33103 |
| UNQ1887 | NM_139015 | 6.66E−07 | 6.29E−07 | 0.394384 | −2.5356 |
| HOMER1 | NM_004272 | 0.001456 | 0.000418 | 0.265524 | −3.76614 |
| LPAR3 | NM_012152 | 1.27E−07 | 5.44E−08 | 0.116731 | −8.56672 |
| LRRC42 | NM_052940 | 0.000315 | 8.76E−05 | 0.385761 | −2.59228 |
| GOLGA8B | NR_027410 | 3.19E−05 | 0.000633 | 0.364663 | −2.74226 |
| CYB5R2 | NM_016229 | 0.000242 | 5.19E−05 | 0.251877 | −3.97019 |
| UBE2F | NM_080678 | 0.0049 | 0.001097 | 0.367286 | −2.72267 |
| TMTC3 | NM_181783 | 2.84E−05 | 9.59E−06 | 0.327532 | −3.05314 |
| ZCCHC11 | NM_001009881 | 0.000345 | 0.000248 | 0.29348 | −3.40739 |
| PPP3CC | NM_005605 | 0.000139 | 3.95E−05 | 0.301889 | −3.31247 |
| SESN3 | NM_144665 | 1.87E−05 | 6.52E−06 | 0.25186 | −3.97047 |
| C14orf149 | NM_144581 | 5.47E−05 | 1.51E−05 | 0.359233 | −2.78371 |
| PTPLA | NM_014241 | 9.50E−07 | 3.15E−07 | 0.308401 | −3.24253 |
| ODF2L | NM_020729 | 5.20E−05 | 0.000116 | 0.359137 | −2.78445 |
| FAM174A | NM_198507 | 0.001283 | 0.000276 | 0.267556 | −3.73754 |
| CBL | NM_005188 | 4.47E−06 | 1.54E−06 | 0.379841 | −2.63268 |
| PDCD1LG2 | NM_025239 | 0.000571 | 0.000181 | 0.25123 | −3.98041 |
| PMAIP1 | NM_021127 | 1.14E−05 | 3.36E−05 | 0.314938 | −3.17523 |
| SACS | NM_014363 | 9.98E−06 | 1.22E−05 | 0.223218 | −4.47992 |
| FKBP14 | NM_017946 | 0.000421 | 0.000142 | 0.318607 | −3.13866 |
| ROBO1 | NM_133631 | 5.83E−07 | 2.71E−07 | 0.096517 | −10.3609 |
| QPCT | NM_012413 | 0.000184 | 9.96E−05 | 0.282185 | −3.54378 |
| ZFP42 | NM_174900 | 0.048358 | 0.018558 | 0.330818 | −3.02281 |
| DSP | NM_004415 | 2.60E−05 | 9.56E−06 | 0.278281 | −3.59349 |
| SPRR1A | NM_005987 | 1.72E−08 | 5.55E−09 | 0.022361 | −44.7214 |
| IL1A | NM_000575 | 8.93E−10 | 3.70E−10 | 0.013557 | −73.7616 |
| LOC654433 | NR_015377 | 0.146494 | 0.039079 | 0.328085 | −3.04799 |
| EPS15 | NM_001981 | 0.013543 | 0.003282 | 0.416586 | −2.40046 |
| S100A11 | NM_005620 | 0.000261 | 6.90E−05 | 0.335729 | −2.97859 |
| SLC36A4 | NM_152313 | 3.46E−05 | 8.13E−06 | 0.21093 | −4.74091 |
| RRAGC | NM_022157 | 0.000313 | 8.26E−05 | 0.418533 | −2.3893 |
| DOCK11 | NM_144658 | 8.05E−07 | 2.49E−07 | 0.122978 | −8.1315 |
| KDSR | NM_002035 | 5.66E−08 | 3.70E−08 | 0.305384 | −3.27456 |
| ERGIC2 | NM_016570 | 0.000598 | 0.000175 | 0.383298 | −2.60894 |
| CSGALNACT2 | NM_018590 | 0.000202 | 0.000104 | 0.376549 | −2.65569 |
| LOC554202 | NR_027054 | 1.47E−07 | 9.82E−08 | 0.19967 | −5.00826 |
| WFDC5 | NM_145652 | 7.11E−06 | 2.73E−06 | 0.119263 | −8.38481 |
| PLXDC2 | NM_032812 | 1.91E−08 | 8.73E−09 | 0.085799 | −11.6551 |
| FBXW7 | NM_033632 | 0.001172 | 0.000341 | 0.377776 | −2.64707 |
| TMEM69 | NM_016486 | 0.000973 | 0.00066 | 0.413698 | −2.41722 |
| TMEM45A | NM_018004 | 1.87E−11 | 8.63E−12 | 0.012327 | −81.123 |
| BBS10 | NM_024685 | 0.001013 | 0.000372 | 0.392586 | −2.54721 |
| SOX2OT | NR_004053 | 0.003922 | 0.723792 | 0.901073 | −1.10979 |
| KDM5B | NM_006618 | 0.00124 | 0.000431 | 0.408709 | −2.44673 |
| CDA | NM_001785 | 7.22E−05 | 3.37E−05 | 0.17196 | −5.8153 |
| IFIT5 | NM_012420 | 0.001922 | 0.000722 | 0.353867 | −2.82592 |
| GTF2H1 | NM_001142307 | 0.000615 | 0.000174 | 0.396649 | −2.52112 |
| NEFM | NM_005382 | 0.000894 | 0.000569 | 0.197269 | −5.06923 |
| SGCE | NM_001099401 | 3.31E−05 | 0.00013 | 0.41879 | −2.38783 |
| DIRC2 | NM_032839 | 3.81E−05 | 9.74E−05 | 0.431249 | −2.31885 |
| ITGA1 | NM_181501 | 3.45E−05 | 2.15E−05 | 0.419243 | −2.38525 |
| RSAD2 | NM_080657 | 0.014966 | 0.007299 | 0.275054 | −3.63566 |
| SLFN5 | NM_144975 | 2.52E−05 | 0.000539 | 0.334263 | −2.99166 |
| SLC2A3 | NM_006931 | 1.19E−05 | 5.47E−06 | 0.107329 | −9.31717 |
| ADAMTS1 | NM_006988 | 8.15E−06 | 6.46E−06 | 0.30871 | −3.23929 |
| ZBTB1 | NM_001123329 | 0.00013 | 3.70E−05 | 0.433489 | −2.30686 |
| PIP5K1A | NM_001135638 | 0.026998 | 0.007491 | 0.363813 | −2.74867 |
| DFNA5 | NM_004403 | 2.70E−06 | 4.22E−06 | 0.205887 | −4.85704 |
| DMKN | NM_033317 | 4.50E−07 | 2.82E−07 | 0.236827 | −4.22248 |
| FLRT3 | NM_198391 | 7.17E−07 | 2.66E−06 | 0.175075 | −5.71182 |
| SPRR3 | NM_005416 | 1.85E−07 | 8.19E−08 | 0.066713 | −14.9897 |
| TTPAL | NM_024331 | 2.86E−06 | 1.31E−06 | 0.264339 | −3.78302 |
| RPS6KA5 | NM_004755 | 4.57E−05 | 1.78E−05 | 0.432782 | −2.31063 |
| CLN5 | NM_006493 | 0.000897 | 0.000251 | 0.442076 | −2.26206 |
| EFEMP1 | NM_004105 | 2.65E−08 | 1.20E−08 | 0.037632 | −26.5735 |
| SLC20A1 | NM_005415 | 6.24E−06 | 2.49E−06 | 0.1965 | −5.08907 |
| GNAI1 | NM_002069 | 0.000112 | 0.000636 | 0.315859 | −3.16597 |
| FERMT1 | NM_017671 | 0.000364 | 0.000113 | 0.412809 | −2.42243 |
| FN1 | NM_212482 | 9.48E−07 | 5.86E−07 | 0.040084 | −24.9475 |
| GJB6 | NM_001110219 | 1.86E−06 | 1.22E−06 | 0.038268 | −26.1318 |
| GPR1 | NM_005279 | 6.76E−05 | 2.62E−05 | 0.430078 | −2.32516 |
| GPR115 | NM_153838 | 1.43E−05 | 6.36E−06 | 0.170203 | −5.87533 |
| ZNF607 | NM_032689 | 4.95E−06 | 1.90E−06 | 0.403938 | −2.47563 |
| MTHFD2L | NM_001144978 | 2.12E−06 | 9.60E−07 | 0.349225 | −2.86348 |
| LRAT | NM_004744 | 1.74E−05 | 8.62E−06 | 0.210638 | −4.74749 |
| C3orf64 | NM_173654 | 0.000232 | 6.09E−05 | 0.290622 | −3.4409 |
| ALDH3B2 | NM_000695 | 0.000138 | 0.000102 | 0.417624 | −2.3945 |
| MT1X | NM_005952 | 0.002024 | 0.000961 | 0.250085 | −3.99863 |
| USP25 | NM_013396 | 0.000737 | 0.0002 | 0.422213 | −2.36847 |
| USP53 | NM_019050 | 0.007056 | 0.002821 | 0.441503 | −2.26499 |
| DUSP6 | NM_001946 | 0.00301 | 0.001557 | 0.414692 | −2.41143 |
| TLE4 | NM_007005 | 5.00E−05 | 0.000131 | 0.426477 | −2.34479 |
| INHBA | NM_002192 | 0.000639 | 0.000228 | 0.283392 | −3.52868 |
| COL12A1 | NM_004370 | 3.22E−07 | 1.88E−07 | 0.089012 | −11.2344 |
| SLIT2 | NM_004787 | 8.48E−06 | 3.43E−06 | 0.316361 | −3.16095 |
| KLF8 | NM_007250 | 1.93E−05 | 6.92E−06 | 0.309101 | −3.23519 |
| IQCA1 | NM_024726 | 3.76E−08 | 1.67E−08 | 0.105066 | −9.5178 |
| BNC1 | NM_001717 | 2.55E−08 | 1.17E−08 | 0.083375 | −11.9941 |
| TCFL5 | NM_006602 | 4.73E−05 | 4.71E−05 | 0.435426 | −2.2966 |
| S100A7 | NM_002963 | 8.46E−06 | 4.68E−06 | 0.015904 | −62.876 |
| EMP3 | NM_001425 | 0.000135 | 7.01E−05 | 0.145977 | −6.85038 |
| DEGS1 | NM_003676 | 0.000529 | 0.00038 | 0.161454 | −6.19372 |
| SPG20 | NM_001142295 | 0.000483 | 0.000236 | 0.383804 | −2.60549 |
| TPD52L1 | NM_001003395 | 1.12E−05 | 5.47E−06 | 0.32833 | −3.04572 |
| GPR137B | NM_003272 | 1.43E−05 | 6.28E−06 | 0.31312 | −3.19367 |
| NIACR2 | NM_006018 | 2.84E−05 | 1.54E−05 | 0.16667 | −5.99987 |
| RBMS3 | NM_001003793 | 0.000283 | 0.000104 | 0.430427 | −2.32327 |
| MUC15 | NM_001135091 | 2.23E−07 | 1.01E−07 | 0.062663 | −15.9583 |
| PPP4R1 | NM_001042388 | 6.81E−05 | 2.25E−05 | 0.376984 | −2.65263 |
| FCHO2 | NM_138782 | 0.041643 | 0.012552 | 0.361248 | −2.76818 |
| LEF1 | NM_016269 | 0.01014 | 0.003991 | 0.394152 | −2.53709 |
| CLASP1 | NM_015282 | 0.000646 | 0.000217 | 0.38302 | −2.61083 |
| TMEM154 | NM_152680 | 3.06E−07 | 1.60E−07 | 0.091846 | −10.8879 |
| IKIP | NM_153687 | 2.32E−08 | 9.14E−09 | 0.100386 | −9.96159 |
| HIVEP2 | NM_006734 | 4.23E−07 | 6.58E−07 | 0.200746 | −4.98141 |
| DSC3 | NM_024423 | 1.04E−10 | 5.14E−11 | 0.01042 | −95.9707 |
| CLDN1 | NM_021101 | 1.30E−06 | 1.03E−05 | 0.383847 | −2.60521 |
| GJB2 | NM_004004 | 3.20E−09 | 1.78E−09 | 0.088101 | −11.3506 |
| WDR47 | NM_001142550 | 5.48E−05 | 2.11E−05 | 0.218538 | −4.57587 |
| SPINK5 | NM_001127698 | 4.21E−06 | 1.42E−06 | 0.061908 | −16.1529 |
| S1PR1 | NM_001400 | 2.02E−06 | 8.79E−07 | 0.194019 | −5.15413 |
| IL1RAP | NM_002182 | 2.28E−07 | 1.32E−07 | 0.17259 | −5.79408 |
| VEGFC | NM_005429 | 2.82E−05 | 1.50E−05 | 0.274021 | −3.64935 |
| AHSA2 | NM_152392 | 0.007404 | 0.007981 | 0.405076 | −2.46867 |
| FBXO3 | NM_033406 | 8.51E−05 | 3.64E−05 | 0.400594 | −2.49629 |
| SRY | NM_003140 | 0.000306 | 0.00011 | 0.25687 | −3.89302 |
| RPSAP52 | NR_026825 | 5.72E−06 | 3.46E−06 | 0.283622 | −3.52582 |
| TAGLN3 | NM_013259 | 7.20E−06 | 4.26E−06 | 0.268918 | −3.71861 |
| BACH1 | NM_206866 | 3.25E−07 | 1.18E−07 | 0.421982 | −2.36977 |
| LY6G6C | NM_025261 | 0.000167 | 6.47E−05 | 0.356472 | −2.80527 |
| ARL17P1 | NM_001113738 | 0.065934 | 0.028718 | 0.39738 | −2.51648 |
| PTGS1 | NM_000962 | 0.000309 | 0.000142 | 0.327626 | −3.05226 |
| NRG1 | NM_013960 | 1.99E−06 | 9.51E−07 | 0.216476 | −4.61945 |
| TCF4 | NM_001083962 | 1.44E−05 | 8.70E−06 | 0.4601 | −2.17344 |
| ZFYVE9 | NM_004799 | 0.00013 | 6.39E−05 | 0.390789 | −2.55892 |
| FAM83A | NM_032899 | 3.35E−06 | 1.35E−06 | 0.291145 | −3.43472 |
| ITGA2 | NM_002203 | 0.000116 | 4.76E−05 | 0.337958 | −2.95894 |
| HERC6 | NM_017912 | 0.000901 | 0.000419 | 0.222522 | −4.49394 |
| FHL1 | NM_001159704 | 2.69E−05 | 1.99E−05 | 0.315264 | −3.17194 |
| USP9Y | NM_004654 | 2.78E−05 | 3.05E−05 | 0.348306 | −2.87103 |
| PLAU | NM_002658 | 0.000318 | 0.000278 | 0.360965 | −2.77035 |
| FGF11 | NM_004112 | 0.000923 | 0.000345 | 0.456957 | −2.18839 |
| CYP4F12 | NM_023944 | 2.48E−06 | 1.28E−05 | 3.4983 | 3.4983 |
| BCAT1 | NM_005504 | 2.88E−05 | 1.70E−05 | 0.225882 | −4.42708 |
| KLK8 | NM_144505 | 3.28E−08 | 1.77E−08 | 0.088545 | −11.2937 |
| BPIL2 | NM_174932 | 3.22E−07 | 1.47E−07 | 0.207224 | −4.8257 |
| GLI3 | NM_000168 | 1.10E−05 | 5.21E−06 | 0.247333 | −4.04314 |
| ZBED2 | NM_024508 | 1.18E−06 | 5.68E−07 | 0.063856 | −15.6602 |
| AADACL2 | NM_207365 | 8.51E−05 | 4.51E−05 | 0.24326 | −4.11083 |
| RHCG | NM_016321 | 0.001734 | 0.000868 | 0.202579 | −4.93635 |
| CCNA1 | NM_003914 | 3.86E−06 | 2.36E−06 | 0.11351 | −8.80982 |
| CA12 | NM_001218 | 8.75E−06 | 2.92E−06 | 0.270851 | −3.69207 |
| S100A12 | NM_005621 | 5.41E−05 | 3.26E−05 | 0.158186 | −6.32166 |
| TP53AIP1 | NM_022112 | 2.01E−06 | 9.95E−07 | 0.270922 | −3.6911 |
| IFNA1 | NM_024013 | 0.00099 | 0.00061 | 0.395885 | −2.52599 |
| DENND2C | NM_198459 | 1.87E−08 | 8.23E−09 | 0.266934 | −3.74625 |
| DSE | NM_013352 | 2.20E−07 | 1.72E−07 | 0.078935 | −12.6686 |
| SLC26A2 | NM_000112 | 0.000313 | 0.000135 | 0.405488 | −2.46616 |
| RECQL | NM_002907 | 0.022406 | 0.009808 | 0.479229 | −2.08668 |
| SERPINB4 | NM_002974 | 1.08E−05 | 5.21E−06 | 0.053201 | −18.7968 |
| UPP1 | NM_003364 | 5.20E−06 | 4.01E−06 | 0.427104 | −2.34135 |
| PTER | NM_030664 | 2.00E−06 | 0.000123 | 2.40143 | 2.40143 |
| IVL | NM_005547 | 1.03E−07 | 4.73E−08 | 0.027985 | −35.7334 |
| GJC1 | NM_005497 | 5.99E−05 | 0.002915 | 0.470474 | −2.12552 |
| SLC2A1 | NM_006516 | 1.53E−07 | 6.53E−08 | 0.257368 | −3.88548 |
| SLC10A6 | NM_197965 | 2.31E−06 | 1.37E−06 | 0.192929 | −5.18324 |
| CLIP1 | NM_002956 | 1.41E−05 | 5.67E−06 | 0.331249 | −3.01888 |
| TPM2 | NM_003289 | 3.73E−05 | 5.11E−05 | 0.264382 | −3.78241 |
| CNTN1 | NM_001843 | 2.39E−09 | 1.41E−09 | 0.043076 | −23.2147 |
| SLC7A5 | NM_003486 | 1.38E−05 | 9.46E−05 | 0.434743 | −2.30021 |
| PAQR7 | NM_178422 | 0.000521 | 0.000873 | 0.4232 | −2.36295 |
| FBLN1 | NM_006486 | 0.000326 | 0.00016 | 0.467469 | −2.13918 |
| SEMA3D | NM_152754 | 0.002343 | 0.000797 | 0.20209 | −4.94828 |
| CCDC3 | NM_031455 | 0.000571 | 0.000331 | 0.264646 | −3.77864 |
| TRAF3IP3 | NM_025228 | 0.000398 | 0.000202 | 0.329368 | −3.03612 |
| NETO1 | NM_138966 | 0.10033 | 0.042964 | 0.478779 | −2.08865 |
| BCO2 | NM_031938 | 6.33E−06 | 3.89E−06 | 0.224959 | −4.44525 |
| AMIGO2 | NM_001143668 | 4.45E−08 | 3.42E−08 | 0.090723 | −11.0225 |
| KRT4 | NM_002272 | 6.63E−07 | 3.56E−07 | 0.078699 | −12.7067 |
| AKTIP | NM_001012398 | 0.006513 | 0.005756 | 0.413823 | −2.41649 |
| SP100 | NM_001080391 | 0.000455 | 0.000268 | 0.261235 | −3.82797 |
| THSD1P | NR_002816 | 0.002072 | 0.001556 | 0.46573 | −2.14717 |
| TMEM136 | NM_174926 | 8.25E−06 | 3.86E−06 | 0.31672 | −3.15736 |
| TTLL7 | NM_024686 | 0.000107 | 4.68E−05 | 0.299448 | −3.33948 |
| RND3 | NM_005168 | 7.33E−05 | 4.65E−05 | 0.434314 | −2.30248 |
| TACSTD2 | NM_002353 | 1.62E−06 | 8.66E−07 | 0.446874 | −2.23777 |
| RBP7 | NM_052960 | 2.56E−05 | 1.85E−05 | 0.148066 | −6.75377 |
| OR10A3 | NM_001003745 | 3.64E−05 | 1.46E−05 | 0.227959 | −4.38675 |
| PLA2R1 | NM_007366 | 2.45E−07 | 2.75E−07 | 0.16495 | −6.06243 |
| KRTDAP | NM_207392 | 1.02E−08 | 6.43E−09 | 0.010491 | −95.3198 |
| PRNP | NM_000311 | 2.18E−06 | 1.34E−06 | 0.274292 | −3.64575 |
| SLC9A9 | NM_173653 | 0.000842 | 0.000526 | 0.428703 | −2.33262 |
| CDC42SE1 | NM_001038707 | 1.84E−05 | 1.06E−05 | 0.366331 | −2.72977 |
| KLK5 | NM_012427 | 9.59E−07 | 5.02E−07 | 0.06538 | −15.2951 |
| KTN1 | NM_182926 | 0.001011 | 0.00053 | 0.462558 | −2.16189 |
| KRT1 | NM_006121 | 6.75E−07 | 3.49E−07 | 0.054981 | −18.1883 |
| RGS20 | NM_170587 | 6.81E−05 | 7.00E−05 | 0.403782 | −2.47659 |
| LHFP | NM_005780 | 9.78E−05 | 5.16E−05 | 0.343621 | −2.91019 |
| C21orf91 | NM_001100420 | 3.26E−05 | 1.17E−05 | 0.193793 | −5.16014 |
| ST3GAL5 | NM_003896 | 1.62E−05 | 7.91E−06 | 0.222702 | −4.49031 |
| KRT24 | NM_019016 | 8.72E−06 | 4.87E−06 | 0.252014 | −3.96804 |
| DSG1 | NM_001942 | 1.97E−11 | 1.13E−11 | 0.006685 | −149.599 |
| PLAT | NM_000930 | 0.001276 | 0.000698 | 0.482639 | −2.07194 |
| THBS2 | NM_003247 | 4.78E−07 | 2.87E−07 | 0.153925 | −6.49666 |
| NIACR1 | NM_177551 | 1.23E−05 | 7.06E−06 | 0.164225 | −6.08919 |
| DSC1 | NM_004948 | 1.38E−08 | 7.85E−09 | 0.031029 | −32.2285 |
| AQP9 | NM_020980 | 0.001541 | 0.000661 | 0.33178 | −3.01404 |
| BNIPL | NM_001159642 | 5.78E−06 | 3.38E−06 | 0.299299 | −3.34114 |
| TNFAIP3 | NM_006290 | 9.56E−05 | 6.56E−05 | 0.495565 | −2.0179 |
| LASS3 | NM_178842 | 3.98E−09 | 2.52E−09 | 0.061733 | −16.1989 |
| RUFY2 | NM_017987 | 3.06E−05 | 2.21E−05 | 0.377722 | −2.64745 |
| SLC26A9 | NM_052934 | 5.61E−07 | 0.000865 | 1.76058 | 1.76058 |
| RORA | NM_134260 | 0.0006 | 0.000263 | 0.452926 | −2.20787 |
| AMOTL1 | NM_130847 | 2.34E−07 | 2.26E−07 | 0.222749 | −4.48937 |
| CARD18 | NM_021571 | 1.65E−06 | 9.58E−07 | 0.101109 | −9.89028 |
| C20orf197 | NM_173644 | 0.012273 | 0.006479 | 0.450831 | −2.21813 |
| CAPN6 | NM_014289 | 1.50E−06 | 0.001521 | 1.97105 | 1.97105 |
| TUBB6 | NM_032525 | 2.26E−06 | 6.75E−06 | 0.365262 | −2.73776 |
| CCDC80 | NM_199511 | 5.69E−06 | 3.25E−06 | 0.178956 | −5.58798 |
| TEX2 | NM_018469 | 1.14E−06 | 5.04E−07 | 0.401033 | −2.49356 |
| EEA1 | NM_003566 | 0.000621 | 0.000249 | 0.391432 | −2.55472 |
| RAET1G | NM_001001788 | 7.86E−06 | 4.61E−06 | 0.111441 | −8.97337 |
| NR3C1 | NM_000176 | 3.04E−05 | 1.74E−05 | 0.432923 | −2.30988 |
| NCF2 | NM_000433 | 4.35E−06 | 2.50E−06 | 0.294316 | −3.39771 |
| TRIML2 | NM_173553 | 0.035133 | 0.018707 | 0.417187 | −2.39701 |
| SLC31A2 | NM_001860 | 7.56E−07 | 3.48E−07 | 0.206476 | −4.84317 |
| ANO4 | NM_178826 | 0.137128 | 0.060183 | 0.449205 | −2.22616 |
| SBSN | NM_198538 | 1.23E−09 | 7.37E−10 | 0.020168 | −49.5844 |
| ELAVL2 | NM_004432 | 4.22E−06 | 2.42E−06 | 0.329714 | −3.03293 |
| BIVM | NM_017693 | 0.000217 | 0.000141 | 0.452207 | −2.21138 |
| LAMC2 | NM_005562 | 1.16E−06 | 6.74E−07 | 0.180854 | −5.52931 |
| PHLDB2 | NM_001134438 | 9.26E−06 | 5.98E−06 | 0.220681 | −4.53142 |
| SFRS12IP1 | NM_173829 | 0.001825 | 0.001164 | 0.429242 | −2.32969 |
| SYT14 | NM_001146261 | 1.09E−07 | 7.04E−08 | 0.084672 | −11.8103 |
| DGKH | NM_178009 | 6.27E−06 | 4.49E−06 | 0.427725 | −2.33795 |
| KRT10 | NM_000421 | 1.21E−09 | 6.90E−10 | 0.02733 | −36.5893 |
| ULK2 | NM_014683 | 3.67E−07 | 3.23E−07 | 0.481301 | −2.0777 |
| DOCK4 | NM_014705 | 1.38E−09 | 8.71E−10 | 0.161512 | −6.1915 |
| CSRNP2 | NM_030809 | 0.00012 | 0.000104 | 0.498398 | −2.00643 |
| LOC284033 | AK095052 | 0.00016 | 7.84E−05 | 0.306697 | −3.26055 |
| DAAM1 | NM_014992 | 6.72E−05 | 3.06E−05 | 0.333974 | −2.99425 |
| HERC5 | NM_016323 | 8.63E−05 | 5.19E−05 | 0.154135 | −6.48784 |
| FGD6 | NM_018351 | 5.97E−06 | 1.78E−05 | 0.33315 | −3.00165 |
| C17orf39 | NM_024052 | 5.60E−05 | 5.97E−05 | 0.463506 | −2.15747 |
| TIPARP | NM_015508 | 7.04E−06 | 7.05E−06 | 0.331273 | −3.01865 |
| ADARB1 | NM_001033049 | 0.000101 | 6.75E−05 | 0.434436 | −2.30183 |
| TLL1 | NM_012464 | 0.000226 | 0.000137 | 0.339309 | −2.94717 |
| EFCAB1 | NM_024593 | 1.04E−07 | 6.34E−08 | 0.23938 | −4.17746 |
| CAMSAP1L1 | NM_203459 | 6.46E−06 | 3.14E−06 | 0.187895 | −5.32212 |
| BMPR2 | NM_001204 | 0.000242 | 0.000155 | 0.359176 | −2.78415 |
| CPA4 | NM_016352 | 9.48E−07 | 5.95E−07 | 0.09492 | −10.5351 |
| UBE2Q2 | NM_173469 | 0.001354 | 0.000658 | 0.439636 | −2.27461 |
| CAB39L | NM_030925 | 1.27E−06 | 7.97E−07 | 0.340316 | −2.93844 |
| TUBA1A | NM_006009 | 0.07469 | 0.032673 | 0.437043 | −2.2881 |
| ORM2 | NM_000608 | 6.33E−06 | 0.073705 | 1.39226 | 1.39226 |
| CLCA2 | NM_006536 | 8.01E−11 | 4.51E−11 | 0.013943 | −71.7201 |
| NIN | NM_020921 | 4.70E−08 | 3.06E−08 | 0.144863 | −6.90308 |
| EML1 | NM_001008707 | 2.17E−06 | 9.65E−07 | 0.330429 | −3.02637 |
| MYO3B | NM_138995 | 0.000197 | 0.000141 | 0.45953 | −2.17613 |
| BBOX1 | NM_003986 | 3.51E−10 | 2.00E−10 | 0.043911 | −22.7735 |
| ZFP36L1 | NM_004926 | 1.83E−05 | 1.81E−05 | 0.456181 | −2.19211 |
| KRT17 | NM_000422 | 5.11E−07 | 2.90E−07 | 0.011104 | −90.0602 |
| EPHA4 | NM_004438 | 6.81E−06 | 2.90E−06 | 0.259308 | −3.85641 |
| ASAP1 | NM_018482 | 5.27E−05 | 3.37E−05 | 0.361779 | −2.76412 |
| PARD6G | NM_032510 | 3.74E−05 | 1.95E−05 | 0.346776 | −2.88371 |
| TUBA4A | NM_006000 | 3.89E−08 | 1.67E−08 | 0.273785 | −3.6525 |
| LOC84740 | NR_026892 | 6.94E−08 | 0.740089 | 1.04468 | 1.04468 |
| TMEM40 | NM_018306 | 9.61E−06 | 6.85E−06 | 0.154403 | −6.47655 |
| ARL14 | NM_025047 | 3.77E−07 | 6.60E−07 | 9.80972 | 9.80972 |
| BTBD11 | NM_001018072 | 2.57E−06 | 1.75E−06 | 0.230868 | −4.33147 |
| SPRR1B | NM_003125 | 6.11E−09 | 2.83E−09 | 0.007571 | −132.083 |
| HIPK3 | NM_005734 | 0.000546 | 0.000271 | 0.414886 | −2.4103 |
| PLS3 | NM_005032 | 1.43E−05 | 7.64E−06 | 0.353785 | −2.82658 |
| SULF2 | NM_018837 | 8.04E−05 | 5.16E−05 | 0.402102 | −2.48693 |
| IGFL2 | NM_001002915 | 1.96E−08 | 1.39E−08 | 0.057 | −17.544 |
| SNAPC1 | NM_003082 | 0.000251 | 0.000323 | 0.441525 | −2.26488 |
| MYO9A | NM_006901 | 0.000169 | 9.69E−05 | 0.466126 | −2.14534 |
| CASP14 | NM_012114 | 6.22E−07 | 3.53E−07 | 0.096227 | −10.3921 |
| LOC100131726 | NR_024479 | 7.07E−07 | 3.36E−07 | 0.142613 | −7.012 |
| TSHZ3 | NM_020856 | 7.41E−06 | 3.91E−06 | 0.096776 | −10.3332 |
| FBXO27 | NM_178820 | 5.13E−05 | 4.66E−05 | 0.424143 | −2.35769 |
| DDX26B | NM_182540 | 2.10E−07 | 2.14E−07 | 0.435689 | −2.29521 |
| IL1F9 | NM_019618 | 0.005257 | 0.003634 | 0.347607 | −2.87682 |
| CSDA | NM_003651 | 6.66E−05 | 0.00011 | 0.473359 | −2.11256 |
| SLC30A4 | NM_013309 | 7.96E−06 | 6.10E−06 | 0.43967 | −2.27443 |
| RAB9A | NM_004251 | 0.000183 | 0.00012 | 0.468324 | −2.13527 |
| DSG4 | NM_001134453 | 2.29E−05 | 1.53E−05 | 0.35497 | −2.81714 |
| MYCBP2 | NM_015057 | 0.000554 | 0.000367 | 0.493869 | −2.02483 |
| STK3 | NM_006281 | 1.09E−05 | 1.22E−05 | 0.341489 | −2.92835 |
| GABRP | NM_014211 | 0.013059 | 0.600955 | 1.24205 | 1.24205 |
| SLC6A11 | NM_014229 | 1.84E−05 | 1.33E−05 | 0.34986 | −2.85829 |
| KRT5 | NM_000424 | 3.19E−09 | 2.07E−09 | 0.021941 | −45.5779 |
| CCL27 | NM_006664 | 0.001975 | 0.001156 | 0.457267 | −2.1869 |
| PTPN14 | NM_005401 | 1.54E−05 | 1.60E−05 | 0.356726 | −2.80327 |
| C3orf34 | NM_032898 | 1.69E−08 | 1.77E−08 | 0.212946 | −4.69603 |
| LAYN | NM_178834 | 1.42E−06 | 1.38E−06 | 0.233102 | −4.28997 |
| NEK1 | NM_012224 | 0.002354 | 0.001458 | 0.381839 | −2.6189 |
| LY6K | NM_017527 | 4.86E−05 | 3.34E−05 | 0.220512 | −4.53491 |
| ULBP1 | NM_025218 | 0.004888 | 0.002621 | 0.240299 | −4.16148 |
| TMPRSS11F | NM_207407 | 4.77E−06 | 2.85E−06 | 0.16775 | −5.96125 |
| GADD45A | NM_001924 | 0.00014 | 0.000159 | 0.413059 | −2.42096 |
| PPP1R14C | NM_030949 | 2.91E−05 | 2.49E−05 | 0.262596 | −3.80814 |
| NAV3 | NM_014903 | 8.29E−06 | 5.74E−06 | 0.326906 | −3.05898 |
| TFPI2 | NM_006528 | 2.13E−11 | 1.56E−11 | 0.027611 | −36.217 |
| SPRR2A | NM_005988 | 3.49E−08 | 1.85E−08 | 0.11895 | −8.40688 |
| CYYR1 | NM_052954 | 0.000156 | 0.000109 | 0.33116 | −3.01969 |
| AQP3 | NM_004925 | 1.81E−08 | 1.03E−08 | 0.19219 | −5.20318 |
| SNCA | NM_000345 | 1.06E−07 | 8.88E−08 | 0.204606 | −4.88745 |
| MORC3 | NM_015358 | 9.63E−06 | 6.22E−06 | 0.417415 | −2.3957 |
| FAT2 | NM_001447 | 2.37E−07 | 1.62E−07 | 0.153144 | −6.5298 |
| PKP1 | NM_000299 | 2.05E−07 | 1.40E−07 | 0.131756 | −7.58976 |
| FEZ1 | NM_005103 | 8.18E−08 | 4.85E−08 | 0.10453 | −9.56661 |
| SFRP1 | NM_003012 | 3.17E−05 | 2.21E−05 | 0.286746 | −3.48741 |
| TGM1 | NM_000359 | 3.34E−07 | 2.70E−07 | 0.157519 | −6.34845 |
| LYST | NM_000081 | 0.006956 | 0.003186 | 0.383653 | −2.60652 |
| HOXC9 | NM_006897 | 8.36E−05 | 6.27E−05 | 0.327183 | −3.05639 |
| SHC1 | NM_183001 | 3.06E−05 | 2.84E−05 | 0.488294 | −2.04795 |
| S100A8 | NM_002964 | 5.73E−10 | 3.66E−10 | 0.004813 | −207.77 |
| GSDMC | NM_031415 | 3.72E−09 | 2.21E−09 | 0.07223 | −13.8447 |
| RAB38 | NM_022337 | 5.10E−09 | 3.71E−09 | 0.165795 | −6.03155 |
| SAA1 | NM_000331 | 0.003802 | 0.003989 | 0.213549 | −4.68277 |
| HERC3 | NM_014606 | 0.000663 | 0.000421 | 0.362275 | −2.76033 |
| FAM127A | NM_001078171 | 1.05E−05 | 6.75E−06 | 0.117546 | −8.50732 |
| FLRT2 | NM_013231 | 7.17E−08 | 5.06E−08 | 0.072015 | −13.8859 |
| PPP4R4 | NM_058237 | 0.000209 | 0.000185 | 0.497693 | −2.00927 |
| INTS6 | NM_012141 | 0.0002 | 0.000152 | 0.423852 | −2.35931 |
| CRCT1 | NM_019060 | 0.000408 | 0.000238 | 0.322439 | −3.10136 |
| DNAJB4 | NM_007034 | 0.000509 | 0.000531 | 0.316077 | −3.16378 |
| ZNF750 | NM_024702 | 3.22E−08 | 2.04E−08 | 0.09617 | −10.3982 |
| HTR7 | NM_019859 | 0.004453 | 0.004553 | 0.497268 | −2.01099 |
| FABP4 | NM_001442 | 0.097674 | 0.056799 | 0.482036 | −2.07453 |
| TNNT2 | NM_000364 | 4.41E−05 | 2.85E−05 | 0.193259 | −5.17441 |
| FER | NM_005246 | 0.000641 | 0.000443 | 0.323276 | −3.09333 |
| GJB4 | NM_153212 | 0.001835 | 0.002072 | 0.499175 | −2.0033 |
| STARD5 | NM_181900 | 4.65E−06 | 3.04E−06 | 0.302556 | −3.30518 |
| DUOXA1 | NM_144565 | 0.000575 | 0.000451 | 0.418438 | −2.38984 |
| SERPINB3 | NM_006919 | 2.94E−08 | 2.00E−08 | 0.012711 | −78.6698 |
| HIAT1 | NM_033055 | 0.01459 | 0.007654 | 0.422788 | −2.36525 |
| MAL | NM_002371 | 7.23E−05 | 0.39336 | 0.893702 | −1.11894 |
| MMP9 | NM_004994 | 0.000388 | 0.000361 | 0.364991 | −2.73979 |
| CD86 | NM_175862 | 0.006281 | 0.006303 | 0.488157 | −2.04852 |
| GM2A | NM_000405 | 8.00E−07 | 6.52E−07 | 0.230115 | −4.34565 |
| NFAT5 | NM_138714 | 3.81E−06 | 5.42E−06 | 0.454936 | −2.19811 |
| AJAP1 | NM_018836 | 1.05E−05 | 9.40E−06 | 0.401521 | −2.49053 |
| CNGA1 | NM_001142564 | 0.045943 | 0.065021 | 0.452679 | −2.20907 |
| OSBPL6 | NM_032523 | 6.84E−08 | 5.06E−08 | 0.167078 | −5.98522 |
| MTSS1 | NM_014751 | 1.35E−08 | 8.38E−09 | 0.207966 | −4.80847 |
| TRIM23 | NM_001656 | 6.72E−06 | 5.22E−06 | 0.393904 | −2.53869 |
| COPZ2 | NM_016429 | 0.000202 | 0.000108 | 0.347427 | −2.87831 |
| C20orf114 | NM_033197 | 1.48E−08 | 0.025151 | 1.28715 | 1.28715 |
| SGTB | NM_019072 | 8.79E−05 | 6.25E−05 | 0.448303 | −2.23063 |
| LYPD3 | NM_014400 | 1.84E−07 | 1.38E−07 | 0.121182 | −8.25202 |
| ALOX15B | NM_001141 | 3.55E−07 | 2.58E−07 | 0.235379 | −4.24847 |
| SLC6A15 | NM_182767 | 2.71E−08 | 1.70E−08 | 0.023514 | −42.5281 |
| MARK3 | NM_001128918 | 1.02E−05 | 1.05E−05 | 0.483147 | −2.06976 |
| BICD2 | NM_001003800 | 6.95E−05 | 5.18E−05 | 0.333536 | −2.99818 |
| PTHLH | NM_198965 | 5.00E−08 | 3.20E−08 | 0.063055 | −15.8592 |
| TPRG1 | NM_198485 | 1.20E−06 | 9.21E−07 | 0.197023 | −5.07554 |
| CYP4F11 | NM_021187 | 5.46E−05 | 4.85E−05 | 0.243507 | −4.10666 |
| PARP9 | NM_001146106 | 0.011648 | 0.007957 | 0.337264 | −2.96504 |
| ITGA5 | NM_002205 | 0.000201 | 0.000206 | 0.452953 | −2.20774 |
| CTSL1 | NM_001912 | 1.13E−05 | 8.72E−06 | 0.289579 | −3.45329 |
| SFN | NM_006142 | 7.85E−09 | 5.23E−09 | 0.273003 | −3.66297 |
| ETNK2 | NM_018208 | 0.000167 | 0.000131 | 0.336163 | −2.97475 |
| SPINK6 | NM_205841 | 1.67E−10 | 9.48E−11 | 0.007065 | −141.545 |
| TFAP2A | NM_003220 | 1.99E−07 | 1.01E−07 | 0.203498 | −4.91405 |
| EMR2 | NM_013447 | 0.00025 | 0.000274 | 0.490212 | −2.03993 |
| CLCA4 | NM_012128 | 1.90E−07 | 1.20E−07 | 0.041414 | −24.1466 |
| S100A9 | NM_002965 | 4.79E−07 | 2.96E−07 | 0.032138 | −31.1161 |
| EPGN | NM_001013442 | 2.48E−08 | 1.59E−08 | 0.024427 | −40.9378 |
| GJB5 | NM_005268 | 1.40E−06 | 6.39E−07 | 0.113731 | −8.79268 |
| MPZL2 | NM_144765 | 8.50E−07 | 5.95E−07 | 0.365191 | −2.7383 |
| NOTCH2 | NM_024408 | 2.38E−06 | 5.20E−06 | 0.456723 | −2.18951 |
| PTPRZ1 | NM_002851 | 1.89E−09 | 1.20E−09 | 0.041355 | −24.1807 |
| KRT14 | NM_000526 | 7.05E−10 | 4.13E−10 | 0.011096 | −90.1236 |
| FAP | NM_004460 | 0.000184 | 0.00014 | 0.249611 | −4.00624 |
| SLC39A2 | NM_014579 | 1.02E−06 | 7.37E−07 | 0.227705 | −4.39164 |
| TMPRSS11E | NM_014058 | 2.32E−05 | 1.49E−05 | 0.068107 | −14.6828 |
| KCNQ5 | NM_019842 | 0.002317 | 0.001959 | 0.467845 | −2.13746 |
| ARL4D | NM_001661 | 1.68E−05 | 1.48E−05 | 0.206539 | −4.84169 |
| PTGS2 | NM_000963 | 0.00066 | 0.000381 | 0.241888 | −4.13415 |
| SIM2 | NM_009586 | 1.67E−06 | 0.10316 | 1.16053 | 1.16053 |
| CDH13 | NM_001257 | 6.12E−08 | 4.66E−08 | 0.073861 | −13.5389 |
| RAB37 | NM_175738 | 4.76E−06 | 0.002977 | 1.43301 | 1.43301 |
| NUAK1 | NM_014840 | 0.001449 | 0.00114 | 0.474631 | −2.1069 |
| ST6GALNAC2 | NM_006456 | 9.39E−08 | 5.37E−08 | 0.172351 | −5.80212 |
| NTM | NM_001144058 | 9.37E−05 | 0.000142 | 0.386502 | −2.58731 |
| PTPRE | NM_006504 | 5.05E−07 | 6.26E−07 | 0.301006 | −3.3222 |
| EMP1 | NM_001423 | 8.78E−06 | 9.68E−06 | 0.264861 | −3.77557 |
| PLD5 | NM_152666 | 1.99E−05 | 1.77E−05 | 0.168198 | −5.94536 |
| GBP6 | NM_198460 | 5.06E−05 | 3.77E−05 | 0.304061 | −3.28882 |
| LAMP2 | NM_002294 | 0.000116 | 0.000115 | 0.340154 | −2.93984 |
| F2R | NM_001992 | 0.000105 | 0.000565 | 0.40176 | −2.48905 |
| PYGL | NM_002863 | 1.53E−08 | 3.26E−08 | 0.167494 | −5.97036 |
| PGLYRP3 | NM_052891 | 0.001406 | 0.001542 | 0.452129 | −2.21176 |
| ORM1 | NM_000607 | 0.000765 | 0.437758 | 1.16127 | 1.16127 |
| LPCAT2 | NM_017839 | 0.00011 | 7.07E−05 | 0.312527 | −3.19972 |
| HOXC10 | NM_017409 | 7.80E−05 | 6.97E−05 | 0.31526 | −3.17198 |
| PLA2G4E | NM_001080490 | 4.00E−08 | 3.36E−08 | 0.177956 | −5.61936 |
| NEBL | NM_006393 | 9.72E−05 | 5.95E−05 | 0.282602 | −3.53855 |
| PCDH21 | NM_033100 | 5.93E−05 | 6.57E−05 | 0.434091 | −2.30367 |
| CALB2 | NM_001740 | 0.000121 | 8.20E−05 | 0.183533 | −5.44861 |
| FSCN1 | NM_003088 | 0.000138 | 0.000192 | 0.465068 | −2.15022 |
| SWAP70 | NM_015055 | 2.00E−07 | 2.12E−07 | 0.371359 | −2.69281 |
| MARK1 | NM_018650 | 1.32E−07 | 1.18E−07 | 0.265896 | −3.76087 |
| IGFL1 | NM_198541 | 4.72E−06 | 3.02E−06 | 0.12375 | −8.0808 |
| KRT77 | NM_175078 | 1.52E−05 | 1.36E−05 | 0.274278 | −3.64593 |
| ERC1 | NM_178037 | 6.44E−06 | 9.84E−06 | 0.49748 | −2.01013 |
| GNAL | NM_182978 | 7.49E−05 | 6.78E−05 | 0.44032 | −2.27108 |
| SERPING1 | NM_000062 | 2.86E−05 | 3.83E−05 | 0.253286 | −3.94811 |
| ATP12A | NM_001676 | 0.000248 | 0.00019 | 0.306484 | −3.26281 |
| LAMP3 | NM_014398 | 0.028786 | 0.019166 | 0.470295 | −2.12632 |
| FST | NM_006350 | 5.16E−07 | 3.36E−07 | 0.124071 | −8.05989 |
| DUOX1 | NM_017434 | 5.36E−05 | 5.29E−05 | 0.396325 | −2.52318 |
| CYP1B1 | NM_000104 | 0.001671 | 0.001644 | 0.398847 | −2.50723 |
| ERCC6 | NM_000124 | 1.12E−08 | 9.06E−09 | 0.241083 | −4.14795 |
| ABCA12 | NM_173076 | 4.61E−09 | 2.31E−09 | 0.019165 | −52.1794 |
| ERCC1 | NM_202001 | 4.26E−05 | 4.26E−05 | 0.362622 | −2.75769 |
| CCDC109B | NM_017918 | 0.002651 | 0.001655 | 0.34527 | −2.89628 |
| TMEM86A | NM_153347 | 5.02E−05 | 6.66E−05 | 0.423458 | −2.36151 |
| KCTD1 | NM_001142730 | 2.89E−07 | 2.43E−07 | 0.354433 | −2.82141 |
| FLJ21511 | NM_025087 | 2.01E−08 | 1.40E−08 | 0.024546 | −40.7406 |
| MSRB3 | NM_001031679 | 0.000156 | 0.00024 | 0.457294 | −2.18678 |
| GATA3 | NM_001002295 | 1.57E−06 | 1.52E−06 | 0.307929 | −3.2475 |
| ETS1 | NM_001143820 | 3.08E−08 | 4.67E−08 | 0.340598 | −2.93602 |
| JUP | NM_002230 | 2.79E−06 | 2.68E−06 | 0.366333 | −2.72976 |
| TAGLN | NM_001001522 | 0.002535 | 0.002146 | 0.44908 | −2.22677 |
| SLC7A1 | NM_003045 | 2.99E−05 | 3.58E−05 | 0.462538 | −2.16198 |
| QKI | NM_206855 | 0.000221 | 0.000276 | 0.446033 | −2.24199 |
| XG | NM_001141919 | 5.23E−06 | 2.82E−06 | 0.147072 | −6.79939 |
| FERMT2 | NM_006832 | 7.03E−07 | 1.34E−06 | 0.243822 | −4.10136 |
| MACF1 | NM_012090 | 3.54E−05 | 3.46E−05 | 0.333934 | −2.9946 |
| OSMR | NM_003999 | 0.000719 | 0.000927 | 0.448172 | −2.23129 |
| GNA15 | NM_002068 | 2.27E−06 | 1.29E−06 | 0.160651 | −6.22468 |
| IFNE | NM_176891 | 1.48E−08 | 9.98E−09 | 0.088706 | −11.2732 |
| AMZ2 | NM_016627 | 5.92E−05 | 9.02E−05 | 0.459625 | −2.17569 |
| TBC1D19 | NM_018317 | 1.69E−05 | 1.96E−05 | 0.431646 | −2.31671 |
| CRIM1 | NM_016441 | 4.50E−07 | 4.71E−07 | 0.312858 | −3.19634 |
| CALML5 | NM_017422 | 2.24E−05 | 2.14E−05 | 0.269774 | −3.7068 |
| GPR64 | NM_001079858 | 3.59E−05 | 0.061676 | 1.38514 | 1.38514 |
| SNX24 | NM_014035 | 0.00317 | 0.002572 | 0.404827 | −2.47019 |
| SERPINB13 | NM_012397 | 2.87E−11 | 1.85E−11 | 0.010222 | −97.826 |
| KRT15 | NM_002275 | 1.07E−09 | 6.99E−10 | 0.035418 | −28.2344 |
| MCC | NM_001085377 | 5.92E−06 | 7.10E−06 | 0.337538 | −2.96263 |
| TP63 | NM_003722 | 1.98E−09 | 1.32E−09 | 0.060277 | −16.59 |
| CYB5R1 | NM_016243 | 8.18E−08 | 5.36E−08 | 0.196948 | −5.07747 |
| SERPINB2 | NM_001143818 | 0.000522 | 0.000316 | 0.130409 | −7.66815 |
| MARVELD1 | NR_026753 | 0.000246 | 0.001632 | 0.499073 | −2.00371 |
| ERRFI1 | NM_018948 | 4.24E−05 | 0.00015 | 0.461638 | −2.1662 |
| SLCO3A1 | NM_013272 | 3.37E−06 | 8.70E−06 | 0.475199 | −2.10438 |
| TIMP1 | NM_003254 | 7.98E−06 | 5.22E−06 | 0.177266 | −5.64125 |
| CAPRIN2 | NM_001002259 | 0.000102 | 0.000198 | 0.43531 | −2.29721 |
| PLTP | NM_006227 | 0.000998 | 0.001704 | 0.473561 | −2.11166 |
| CALCRL | NM_005795 | 7.23E−07 | 2.74E−06 | 0.465558 | −2.14796 |
| IFIH1 | NM_022168 | 0.015725 | 0.0111 | 0.37925 | −2.63678 |
| CLIC4 | NM_013943 | 0.001914 | 0.002345 | 0.482499 | −2.07254 |
| IRF6 | NM_006147 | 2.28E−07 | 2.73E−07 | 0.274871 | −3.63807 |
| A2ML1 | NM_144670 | 7.47E−08 | 4.30E−08 | 0.012286 | −81.3962 |
| FCHSD2 | NM_014824 | 3.04E−05 | 2.74E−05 | 0.342655 | −2.91839 |
| DNAJB5 | NM_001135005 | 0.0014 | 0.002946 | 0.450186 | −2.2213 |
| TIAM1 | NM_003253 | 1.18E−06 | 1.01E−06 | 0.280659 | −3.56304 |
| CAPNS2 | NM_032330 | 1.59E−07 | 1.38E−07 | 0.028316 | −35.3156 |
| KATNAL1 | NM_001014380 | 1.94E−06 | 2.23E−06 | 0.220371 | −4.53781 |
| GRHL3 | NM_198173 | 3.64E−09 | 3.70E−09 | 0.247268 | −4.04419 |
| MAP2 | NM_002374 | 1.28E−07 | 1.26E−07 | 0.251667 | −3.97351 |
| SMARCA1 | NM_003069 | 3.35E−05 | 0.00021 | 0.459756 | −2.17507 |
| C9orf95 | NR_023352 | 0.00091 | 0.001364 | 0.477435 | −2.09453 |
| LUM | NM_002345 | 0.00038 | 0.0381 | 1.75631 | 1.75631 |
| MLF1 | NM_001130157 | 0.000152 | 0.000314 | 0.434315 | −2.30248 |
| RPE65 | NM_000329 | 0.004304 | 0.009863 | 0.482605 | −2.07209 |
| KLF7 | NM_003709 | 3.34E−07 | 3.44E−07 | 0.281847 | −3.54802 |
| STEAP4 | NM_024636 | 4.23E−09 | 3.62E−09 | 0.067452 | −14.8253 |
| ARSJ | NM_024590 | 3.70E−05 | 6.21E−05 | 0.408595 | −2.44741 |
| FGF5 | NM_004464 | 0.000358 | 0.000318 | 0.281323 | −3.55463 |
| IFI44L | NM_006820 | 0.001777 | 0.001409 | 0.093098 | −10.7414 |
| TNC | NM_002160 | 3.71E−06 | 3.69E−06 | 0.229225 | −4.36253 |
| LY6D | NM_003695 | 0.00028 | 0.00047 | 0.391998 | −2.55103 |
| SLITRK6 | NM_032229 | 0.00074 | 0.000631 | 0.266593 | −3.75104 |
| RAET1E | NM_139165 | 3.95E−06 | 4.49E−06 | 0.217168 | −4.60473 |
| SEC14L2 | NM_012429 | 2.11E−06 | 3.38E−06 | 0.399713 | −2.5018 |
| DUSP7 | NM_001947 | 3.65E−06 | 8.31E−06 | 0.46662 | −2.14307 |
| ELK3 | NM_005230 | 1.44E−06 | 2.42E−06 | 0.300748 | −3.32504 |
| SMURF2 | NM_022739 | 8.79E−06 | 2.18E−05 | 0.451238 | −2.21612 |
| TRIM29 | NM_012101 | 1.30E−08 | 9.48E−09 | 0.137993 | −7.24674 |
| UGT1A9 | NM_021027 | 3.69E−06 | 0.008457 | 0.673124 | −1.48561 |
| | — | 0.017796 | 0.034272 | 0.41183 | −2.42819 |
| SERPINE1 | NM_000602 | 0.000464 | 0.000338 | 0.187171 | −5.3427 |
| MYO5A | NM_000259 | 6.81E−10 | 6.71E−10 | 0.098157 | −10.1878 |
| | — | 1.57E−06 | 1.47E−06 | 0.184668 | −5.41512 |
| EGFR | NM_005228 | 7.76E−08 | 1.17E−07 | 0.289142 | −3.45851 |
| SLC38A2 | NM_018976 | 7.02E−08 | 7.65E−08 | 0.288013 | −3.47207 |
| HAS2 | NM_005328 | 0.004297 | 0.007023 | 0.488528 | −2.04697 |
| LRRC8C | NM_032270 | 1.86E−05 | 4.09E−05 | 0.253481 | −3.94506 |
| MPDZ | NM_003829 | 0.001944 | 0.006307 | 0.455314 | −2.19629 |
| DDX60 | NM_017631 | 0.006426 | 0.009508 | 0.325216 | −3.07488 |
| PCDHB2 | NM_018936 | 0.000695 | 0.175936 | 1.2872 | 1.2872 |
| IL1B | NM_000576 | 5.25E−07 | 3.89E−07 | 0.10028 | −9.97204 |
| BBS9 | NM_198428 | 0.003029 | 0.00491 | 0.471346 | −2.12158 |
| STEAP1 | NM_012449 | 0.135915 | 0.156455 | 0.419955 | −2.38121 |
| CD274 | NM_014143 | 5.19E−05 | 7.83E−05 | 0.361298 | −2.7678 |
| SLC39A6 | NM_012319 | 3.85E−07 | 4.72E−07 | 0.255285 | −3.91719 |
| MGAM | NM_004668 | 1.99E−07 | 0.000115 | 1.54142 | 1.54142 |
| SEMA3C | NM_006379 | 0.000153 | 0.000259 | 0.394099 | −2.53744 |
| WDFY2 | NM_052950 | 2.45E−08 | 6.49E−08 | 0.38159 | −2.62061 |
| LDOC1 | NM_012317 | 1.18E−05 | 2.88E−05 | 0.374132 | −2.67285 |
| GLTP | NM_016433 | 0.000199 | 0.000481 | 0.429942 | −2.3259 |
| CAPN13 | NM_144575 | 1.06E−07 | 7.76E−06 | 1.96105 | 1.96105 |
| IKZF2 | NM_001079526 | 1.78E−06 | 2.80E−06 | 0.309067 | −3.23554 |
| RBP1 | NM_001130992 | 1.32E−06 | 0.001574 | 0.470568 | −2.12509 |
| SCGB2A1 | NM_002407 | 4.48E−06 | 0.059586 | 1.31277 | 1.31277 |
| IGFBP6 | NM_002178 | 6.93E−06 | 1.33E−05 | 0.219911 | −4.54729 |
| C7orf10 | NM_024728 | 1.87E−07 | 8.84E−07 | 0.437612 | −2.28513 |
| SLPI | NM_003064 | 1.62E−06 | 1.44E−06 | 0.127431 | −7.84737 |
| CD109 | NM_133493 | 9.85E−09 | 7.72E−09 | 0.072182 | −13.8539 |
| SP110 | NM_080424 | 0.002794 | 0.005834 | 0.475861 | −2.10145 |
| VGLL1 | NM_016267 | 0.000107 | 0.00025 | 0.261018 | −3.83115 |
| LRP12 | NM_013437 | 1.05E−06 | 2.43E−06 | 0.334472 | −2.98979 |
| PRB4 | NM_002723 | 0.023507 | 0.028814 | 0.366051 | −2.73186 |
| OPTN | NM_001008211 | 1.79E−05 | 6.64E−05 | 0.471762 | −2.11971 |
| YPEL5 | NM_001127401 | 0.000254 | 0.000762 | 0.476714 | −2.09769 |
| SULT2B1 | NM_004605 | 7.72E−05 | 0.000455 | 0.474461 | −2.10766 |
| CDH3 | NM_001793 | 7.22E−06 | 3.22E−05 | 0.412099 | −2.4266 |
| MLLT11 | NM_006818 | 7.84E−05 | 0.00014 | 0.167583 | −5.9672 |
| DRAP1 | NM_006442 | 0.000223 | 0.000666 | 0.465382 | −2.14877 |
| CASP1 | NM_033292 | 1.68E−06 | 6.26E−06 | 0.197904 | −5.05296 |
| TFAP2C | NM_003222 | 7.94E−06 | 2.51E−05 | 0.434692 | −2.30048 |
| EREG | NM_001432 | 0.000459 | 0.00082 | 0.215688 | −4.63633 |
| CAV1 | NM_001753 | 3.96E−08 | 5.88E−08 | 0.095451 | −10.4766 |
| OGFRL1 | NM_024576 | 8.46E−06 | 1.81E−05 | 0.240403 | −4.15968 |
| DEFB1 | NM_005218 | 1.17E−05 | 1.07E−05 | 0.125584 | −7.96278 |
| MRAP2 | NM_138409 | 1.35E−07 | 5.55E−06 | 2.6499 | 2.6499 |
| KRT6A | NM_005554 | 9.88E−08 | 6.23E−08 | 0.019287 | −51.8491 |
| FDXACB1 | NM_138378 | 5.58E−06 | 4.87E−06 | 0.058653 | −17.0494 |
| PI3 | NM_002638 | 2.91E−05 | 0.000337 | 0.324725 | −3.07953 |
| FZD6 | NM_003506 | 0.00022 | 0.001103 | 0.488675 | −2.04635 |
| SPTLC3 | NM_018327 | 1.08E−05 | 5.58E−05 | 0.396875 | −2.51968 |
| CLIP4 | NM_024692 | 1.46E−05 | 5.76E−05 | 0.307238 | −3.2548 |
| RAB31 | NM_006868 | 1.73E−06 | 4.05E−06 | 0.201838 | −4.95448 |
| KLK13 | NM_015596 | 2.92E−05 | 7.82E−05 | 0.365322 | −2.73731 |
| CD44 | NM_000610 | 6.59E−06 | 0.000944 | 0.464831 | −2.15132 |
| DZIP1 | NM_198968 | 3.02E−06 | 4.18E−05 | 0.436947 | −2.28861 |
| | — | 0.010603 | 0.021757 | 0.467854 | −2.13742 |
| CALD1 | NM_033138 | 1.59E−05 | 6.56E−05 | 0.290067 | −3.44748 |
| TUBG2 | NM_016437 | 7.33E−06 | 6.60E−05 | 0.471308 | −2.12176 |
| PRKCH | NM_006255 | 2.73E−05 | 0.000208 | 0.477771 | −2.09305 |
| KRT16 | NM_005557 | 3.75E−08 | 2.92E−08 | 0.016898 | −59.18 |
| FAM63B | NM_001040450 | 1.96E−05 | 6.06E−05 | 0.274221 | −3.64669 |
| C3orf67 | BC132815 | 3.15E−07 | 2.16E−06 | 0.426573 | −2.34426 |
| RIMKLB | NM_020734 | 1.58E−05 | 3.10E−05 | 0.275478 | −3.63005 |
| ATP10D | NM_020453 | 1.04E−06 | 1.38E−06 | 0.156271 | −6.39915 |
| ARL4C | NM_005737 | 8.07E−07 | 1.61E−06 | 0.264687 | −3.77805 |
| FRMD6 | NM_001042481 | 5.92E−07 | 8.16E−07 | 0.15212 | −6.57374 |
| KRT13 | NM_153490 | 2.54E−07 | 2.72E−07 | 0.039864 | −25.0852 |
| KIF3A | NM_007054 | 0.006094 | 0.01185 | 0.360572 | −2.77337 |
| FBP2 | NM_003837 | 6.19E−06 | 0.000707 | 2.038 | 2.038 |
| PHLDB2 | NM_001134438 | 2.38E−06 | 4.18E−06 | 0.181124 | −5.52107 |
| SNAI2 | NM_003068 | 4.56E−08 | 7.90E−08 | 0.039535 | −25.2942 |
| IFIT1 | NM_001548 | 0.000118 | 0.000184 | 0.078991 | −12.6596 |
| SCEL | NM_144777 | 7.26E−07 | 1.49E−06 | 0.135947 | −7.3558 |
| PITPNC1 | NM_181671 | 4.67E−08 | 2.43E−07 | 0.337134 | −2.96618 |
| DDX58 | NM_014314 | 1.91E−05 | 5.21E−05 | 0.265773 | −3.76262 |
| ITGBL1 | NM_004791 | 1.75E−05 | 0.003058 | 2.23812 | 2.23812 |
| PYGB | NM_002862 | 7.79E−06 | 9.30E−05 | 0.48814 | −2.04859 |
| CAV2 | NM_001233 | 2.37E−05 | 0.000191 | 0.353143 | −2.83172 |
| DCBLD2 | NM_080927 | 1.79E−07 | 5.13E−07 | 0.261835 | −3.8192 |
| PALMD | NM_017734 | 8.09E−09 | 2.20E−08 | 0.191061 | −5.23394 |
| EPHX3 | NM_024794 | 0.007575 | 0.044958 | 0.495538 | −2.01801 |
| UGT2B15 | NM_001076 | 8.86E−05 | 0.001024 | 4.48116 | 4.48116 |
| CYBRD1 | NM_024843 | 7.67E−07 | 1.17E−06 | 0.143509 | −6.96818 |
| STXBP1 | NM_003165 | 1.67E−06 | 2.62E−05 | 0.408365 | −2.44879 |
| IFIT3 | NM_001031683 | 0.012789 | 0.047085 | 0.401431 | −2.49109 |
| PLK2 | NM_006622 | 4.19E−06 | 3.34E−05 | 0.314953 | −3.17508 |
| ATP2B4 | NM_001001396 | 2.62E−06 | 1.20E−05 | 0.316108 | −3.16347 |
| MID2 | NM_012216 | 1.44E−07 | 1.55E−06 | 0.396467 | −2.52228 |
| CCL28 | NM_148672 | 9.94E−05 | 1.19E−05 | 4.73086 | 4.73086 |
| ZNF185 | NM_007150 | 9.88E−08 | 8.44E−07 | 0.370874 | −2.69634 |
| USP44 | NM_032147 | 3.67E−05 | 1.13E−05 | 2.46786 | 2.46786 |
| STC2 | NM_003714 | 0.007593 | 0.001551 | 2.71205 | 2.71205 |
| ANXA1 | NM_000700 | 1.81E−05 | 0.000396 | 0.496828 | −2.01277 |
| DAPP1 | NM_014395 | 6.66E−07 | 4.14E−06 | 0.334232 | −2.99194 |
| TCP11L1 | NM_018393 | 1.16E−07 | 1.36E−06 | 0.398607 | −2.50873 |
| PIK3C2G | NM_004570 | 1.19E−05 | 0.005384 | 1.97156 | 1.97156 |
| ITGB6 | NM_000888 | 1.44E−05 | 0.000105 | 0.35966 | −2.7804 |
| IFI6 | NM_002038 | 0.000558 | 0.002824 | 0.370478 | −2.69922 |
| AREG | NM_001657 | 9.80E−08 | 2.46E−07 | 0.147063 | −6.79982 |
| TCEA3 | NM_003196 | 6.03E−05 | 0.004343 | 1.89648 | 1.89648 |
| NKX6-3 | NM_152568 | 0.000222 | 4.36E−05 | 2.62399 | 2.62399 |
| CRABP2 | NM_001878 | 1.24E−09 | 1.60E−09 | 0.070954 | −14.0936 |
| NEXN | NM_144573 | 0.000501 | 0.010025 | 0.433806 | −2.30518 |
| HSPC159 | NM_014181 | 7.31E−08 | 5.29E−07 | 0.320174 | −3.1233 |
| SAMD9L | NM_152703 | 0.002066 | 0.020527 | 0.481049 | −2.07879 |
| TNS4 | NM_032865 | 1.33E−06 | 1.11E−05 | 0.309366 | −3.23242 |
| PTPN13 | NM_080683 | 2.15E−06 | 5.85E−06 | 0.143409 | −6.97308 |
| SERPINB7 | NM_003784 | 5.70E−08 | 7.81E−08 | 0.027398 | −36.4991 |
| PSCA | NM_005672 | 6.84E−07 | 0.000149 | 2.73319 | 2.73319 |
| NPSR1 | NM_207172 | 2.73E−06 | 3.99E−05 | 3.10457 | 3.10457 |
| CTH | NM_001902 | 0.000612 | 8.41E−05 | 3.8776 | 3.8776 |
| MX1 | NM_001144925 | 0.000642 | 0.004132 | 0.296965 | −3.3674 |
| LRRC6 | NM_012472 | 0.002159 | 0.000568 | 3.03333 | 3.03333 |
| TNFRSF10C | NM_003841 | 7.46E−05 | 1.74E−05 | 4.07284 | 4.07284 |
| CYR61 | NM_001554 | 4.65E−05 | 0.002536 | 0.486456 | −2.05568 |
| CXCL17 | NM_198477 | 1.48E−06 | 0.686453 | 1.10161 | 1.10161 |
| ANKRD50 | NM_020337 | 1.33E−05 | 0.000643 | 0.479385 | −2.08601 |
| GSTM4 | NM_000850 | 1.62E−06 | 2.29E−07 | 15.5494 | 15.5494 |
| GSTM2 | NM_000848 | 0.000898 | 0.000207 | 3.39662 | 3.39662 |
| HRASLS2 | NM_017878 | 0.000251 | 0.009109 | 2.71842 | 2.71842 |
| C11orf92 | NM_207429 | 9.79E−08 | 8.14E−07 | 4.28703 | 4.28703 |
| ODAM | NM_017855 | 6.17E−06 | 9.19E−07 | 21.2503 | 21.2503 |
| AHNAK2 | NM_138420 | 8.07E−08 | 6.68E−07 | 0.20488 | −4.88091 |
| DDX43 | NM_018665 | 0.000328 | 6.84E−05 | 5.61594 | 5.61594 |
| IFI16 | NM_005531 | 1.79E−06 | 5.60E−06 | 0.106816 | −9.36187 |
| SLC16A4 | NM_004696 | 0.000184 | 0.00011 | 10.6066 | 10.6066 |
| AK5 | NM_174858 | 0.000101 | 1.64E−05 | 5.2323 | 5.2323 |
| FKBP5 | NM_001145775 | 8.41E−05 | 0.001373 | 0.314812 | −3.1765 |
| THBS1 | NM_003246 | 6.21E−05 | 0.000356 | 0.188047 | −5.31782 |
| KCNJ15 | NM_002243 | 5.99E−07 | 0.000208 | 0.498867 | −2.00454 |
| LCN2 | NM_005564 | 4.89E−05 | 0.000961 | 0.309656 | −3.22939 |
| HS3ST5 | NM_153612 | 7.36E−05 | 1.83E−05 | 3.97874 | 3.97874 |
| CAPN9 | NM_006615 | 1.80E−09 | 9.82E−08 | 4.95981 | 4.95981 |
| CLDN10 | NM_182848 | 1.13E−06 | 4.61E−07 | 3.6212 | 3.6212 |
| KLK10 | NM_002776 | 2.17E−06 | 0.000336 | 0.448689 | −2.22871 |
| SAMD9 | NM_017654 | 8.12E−06 | 4.59E−05 | 0.138963 | −7.19618 |
| HLA-DMB | NM_002118 | 0.000348 | 8.26E−05 | 4.70494 | 4.70494 |
| KLK7 | NM_139277 | 5.21E−07 | 8.75E−06 | 0.190983 | −5.23607 |
| NTS | NM_006183 | 0.018973 | 0.0031 | 9.28925 | 9.28925 |
| TGFB2 | NM_001135599 | 0.001966 | 0.000761 | 3.65589 | 3.65589 |
| CYP2E1 | NM_000773 | 3.35E−05 | 1.79E−05 | 3.40286 | 3.40286 |
| ALDH3A1 | NM_000691 | 1.89E−08 | 1.95E−07 | 5.15825 | 5.15825 |
| CCBE1 | NM_133459 | 4.81E−06 | 1.96E−05 | 2.82626 | 2.82626 |
| MATN2 | NM_002380 | 6.77E−06 | 3.60E−05 | 2.61679 | 2.61679 |
| MFAP5 | NM_003480 | 3.58E−05 | 0.000104 | 0.059705 | −16.7491 |
| BAAT | NM_001701 | 5.52E−08 | 9.52E−09 | 7.07911 | 7.07911 |
| SLC15A1 | NM_005073 | 4.59E−06 | 3.94E−06 | 3.51845 | 3.51845 |
| MXRA5 | NM_015419 | 0.000382 | 0.0001 | 5.60412 | 5.60412 |
| FGF2 | NM_002006 | 4.92E−06 | 1.56E−06 | 5.5274 | 5.5274 |
| IFI44 | NM_006417 | 0.000107 | 0.000873 | 0.128059 | −7.80893 |
| CSTA | NM_005213 | 2.09E−07 | 7.47E−07 | 0.024791 | −40.3374 |
| SERPINB5 | NM_002639 | 1.66E−09 | 7.33E−08 | 0.136954 | −7.30173 |
| GPR87 | NM_023915 | 1.16E−07 | 4.53E−06 | 0.135101 | −7.40189 |
| BICC1 | NM_001080512 | 2.71E−06 | 6.08E−07 | 14.6863 | 14.6863 |
| MSN | NM_002444 | 2.62E−07 | 0.000796 | 0.429086 | −2.33053 |
| GKN1 | NM_019617 | 1.12E−07 | 6.54E−07 | 37.4703 | 37.4703 |
| GKN2 | NM_182536 | 1.22E−08 | 4.25E−08 | 53.4059 | 53.4059 |

Expression microarrays were used to compare the mRNA expression of an isolated clonal population of Barrett's esophagus progenitor cells and a clonal population of gastric cardia progenitor cells. The results of this comparison are shown in Table YY, below.

TABLE YY

| | | | p-value | Ratio | Fold-Change |
|---|---|---|---|---|---|
| | | p-value | (Barrett's vs. | (Barrett's vs. | (Barrett's vs. |
| Gene Symbol | RefSeq | (Attribute) | Cardia) | Cardia) | Cardia) |
| FABP1 | NM_001443 | 2.34E−09 | 0.443141 | 1.1736 | 1.1736 |
| CPS1 | NM_001122633 | 2.60E−07 | 0.721073 | 1.09633 | 1.09633 |
| FABP2 | NM_000134 | 5.39E−08 | 0.915975 | 0.979547 | −1.02088 |
| PRSS2 | NM_002770 | 2.27E−05 | 0.122173 | 0.53986 | −1.85233 |
| KRT20 | NM_019010 | 5.33E−07 | 0.135603 | 0.680989 | −1.46845 |
| DMBT1 | NM_007329 | 7.79E−09 | 0.927446 | 0.987845 | −1.0123 |
| SI | NM_001041 | 8.23E−09 | 0.863031 | 1.02302 | 1.02302 |
| MTTP | NM_000253 | 2.97E−09 | 0.39728 | 1.09805 | 1.09805 |
| RBP2 | NM_004164 | 2.08E−07 | 0.364822 | 1.18689 | 1.18689 |
| MT1H | NM_005951 | 2.21E−06 | 1.31E−05 | 0.121771 | −8.21216 |
| CLCA1 | NM_001285 | 1.94E−07 | 0.674392 | 0.932292 | −1.07263 |
| KGFLP2 | NR_003670 | 7.43E−06 | 0.122882 | 0.668756 | −1.49531 |
| GUCY2C | NM_004963 | 3.89E−09 | 0.289294 | 1.12138 | 1.12138 |
| GSTA2 | NM_000846 | 0.000164 | 0.651187 | 1.24989 | 1.24989 |
| CDH17 | NM_004063 | 3.78E−09 | 1.82E−06 | 6.74546 | 6.74546 |
| C17orf78 | NM_173625 | 0.00023 | 0.348551 | 0.710482 | −1.40749 |
| GPR128 | NM_032787 | 3.01E−08 | 0.20003 | 1.17444 | 1.17444 |
| TM4SF4 | NM_004617 | 1.49E−08 | 0.005923 | 1.86836 | 1.86836 |
| GJA1 | NM_000165 | 0.000666 | 0.195597 | 0.503858 | −1.98469 |
| OTC | NM_000531 | 1.40E−07 | 0.001647 | 2.15821 | 2.15821 |
| BEX1 | NM_018476 | 3.43E−05 | 0.912943 | 0.972302 | −1.02849 |
| HIST1H1A | NM_005325 | 1.95E−07 | 0.041247 | 1.42195 | 1.42195 |
| OLFM4 | NM_006418 | 1.75E−10 | 2.00E−08 | 9.46554 | 9.46554 |
| LOC29034 | NR_002763 | 1.07E−07 | 0.973418 | 1.00379 | 1.00379 |
| BTNL3 | NM_197975 | 4.86E−06 | 0.389088 | 1.19403 | 1.19403 |
| DPY19L2P2 | NR_003561 | 0.000999 | 0.326134 | 0.669634 | −1.49335 |
| CPE | NM_001873 | 1.65E−06 | 0.936389 | 0.988612 | −1.01152 |
| RGS5 | NM_003617 | 1.02E−05 | 0.010102 | 0.499497 | −2.00202 |
| CPVL | NM_019029 | 1.05E−06 | 0.006054 | 0.644326 | −1.55201 |
| DSG3 | NM_001944 | 7.14E−10 | 0.468093 | 1.09383 | 1.09383 |
| TM4SF20 | NM_024795 | 3.07E−07 | 0.964612 | 1.00533 | 1.00533 |
| SLC38A11 | NM_173512 | 2.05E−06 | 0.498316 | 1.11012 | 1.11012 |
| ADH4 | NM_000670 | 2.50E−07 | 0.054095 | 1.32233 | 1.32233 |
| CEACAM6 | NM_002483 | 3.08E−05 | 0.000582 | 10.7156 | 10.7156 |
| SYNPR | NM_001130003 | 2.23E−05 | 0.877069 | 1.03258 | 1.03258 |
| ALDOB | NM_000035 | 2.00E−07 | 0.000338 | 2.61325 | 2.61325 |
| FAM13A | NM_001015045 | 2.05E−05 | 0.250942 | 0.792478 | −1.26186 |
| SLC17A4 | NM_005495 | 4.81E−06 | 0.740182 | 1.0575 | 1.0575 |
| CACNA2D1 | NM_000722 | 9.75E−08 | 0.203122 | 1.15551 | 1.15551 |
| ATF7IP2 | NM_024997 | 2.12E−05 | 0.002184 | 0.461398 | −2.16733 |
| MEP1A | NM_005588 | 1.31E−06 | 0.680298 | 1.0605 | 1.0605 |
| RBM46 | NM_144979 | 7.01E−05 | 0.895065 | 0.972392 | −1.02839 |
| ZG16 | NM_152338 | 8.01E−05 | 0.802539 | 1.05959 | 1.05959 |
| REG4 | NM_001159352 | 2.83E−08 | 0.15174 | 0.785147 | −1.27365 |
| MUC17 | NM_001040105 | 1.67E−06 | 0.000237 | 4.49768 | 4.49768 |
| LGR5 | NM_003667 | 3.55E−07 | 0.413975 | 1.11663 | 1.11663 |
| PRSS1 | NM_002769 | 8.44E−05 | 0.006798 | 0.43254 | −2.31192 |
| SLC2A2 | NM_000340 | 3.42E−06 | 0.644334 | 1.07519 | 1.07519 |
| PHYHIPL | NM_032439 | 1.38E−05 | 0.779037 | 0.949399 | −1.0533 |
| ACE2 | NM_021804 | 1.43E−07 | 0.026458 | 1.34995 | 1.34995 |
| CCND2 | NM_001759 | 5.19E−05 | 0.627003 | 0.870802 | −1.14837 |
| SULT1E1 | NM_005420 | 3.45E−07 | 0.484764 | 1.13106 | 1.13106 |
| SLC5A1 | NM_000343 | 8.44E−06 | 0.063502 | 1.57083 | 1.57083 |
| SEMA6A | NM_020796 | 7.77E−07 | 0.001034 | 2.24175 | 2.24175 |
| MT1L | NR_001447 | 0.004952 | 0.030826 | 0.218511 | −4.57642 |
| HMGCS2 | NM_005518 | 3.07E−07 | 0.004662 | 0.569986 | −1.75443 |
| MGAT4A | NM_012214 | 6.97E−06 | 0.241395 | 0.76894 | −1.30049 |
| UGT2B17 | NM_001077 | 5.68E−06 | 0.509228 | 1.12953 | 1.12953 |
| C15orf48 | NM_032413 | 1.19E−08 | 0.048232 | 1.20649 | 1.20649 |
| CISD2 | NM_001008388 | 0.000711 | 0.120867 | 0.691153 | −1.44686 |
| SST | NM_001048 | 0.000975 | 0.862 | 1.04966 | 1.04966 |
| SPC25 | NM_020675 | 0.033633 | 0.163262 | 0.547071 | −1.82792 |
| PLA2G12B | NM_032562 | 1.29E−05 | 0.818515 | 0.967277 | −1.03383 |
| LGALS2 | NM_006498 | 1.72E−08 | 0.002572 | 1.42912 | 1.42912 |
| NR1H4 | NM_005123 | 5.91E−06 | 0.994737 | 1.00093 | 1.00093 |
| UGT3A1 | NM_152404 | 1.50E−05 | 0.373038 | 0.874674 | −1.14328 |
| GIP | NM_004123 | 0.066104 | 0.974579 | 1.01786 | 1.01786 |
| LOC147727 | NR_024333 | 1.58E−05 | 0.819467 | 0.970185 | −1.03073 |
| ABCG2 | NM_004827 | 0.000813 | 0.308928 | 0.801872 | −1.24708 |
| OCR1 | AF314543 | 0.024574 | 0.35436 | 1.59119 | 1.59119 |
| LMBR1 | NM_022458 | 0.008559 | 0.417077 | 0.641549 | −1.55873 |
| A1CF | NM_138933 | 7.40E−07 | 0.000107 | 3.13037 | 3.13037 |
| IGF2BP1 | NM_006546 | 1.50E−07 | 0.643519 | 1.03984 | 1.03984 |
| TSPAN7 | NM_004615 | 0.000601 | 0.992739 | 1.00201 | 1.00201 |
| CEACAM7 | NM_006890 | 3.74E−06 | 0.853763 | 1.02232 | 1.02232 |
| MYB | NM_001130173 | 4.65E−06 | 0.040136 | 0.683024 | −1.46408 |
| CFI | NM_000204 | 8.87E−06 | 0.01031 | 1.91938 | 1.91938 |
| SLC10A2 | NM_000452 | 6.69E−05 | 0.836931 | 1.03668 | 1.03668 |
| UGT2A3 | NR_024010 | 1.48E−07 | 6.93E−06 | 5.52838 | 5.52838 |
| IFITM1 | NM_003641 | 6.18E−05 | 0.237116 | 1.39004 | 1.39004 |
| TMEM20 | NM_001134658 | 0.000102 | 0.600376 | 1.0965 | 1.0965 |
| TNFRSF11B | NM_002546 | 1.34E−05 | 0.396002 | 1.18421 | 1.18421 |
| SMOC2 | NM_022138 | 8.35E−05 | 0.680888 | 1.07905 | 1.07905 |
| TGFBI | NM_000358 | 0.000306 | 0.525043 | 0.81542 | −1.22636 |
| GPA33 | NM_005814 | 0.00014 | 0.753218 | 1.06031 | 1.06031 |
| NELL2 | NM_001145108 | 4.57E−05 | 0.587981 | 1.0888 | 1.0888 |
| ATP1B3 | NM_001679 | 7.11E−07 | 0.004315 | 0.593605 | −1.68462 |
| FGF9 | NM_002010 | 2.98E−05 | 0.821578 | 0.969452 | −1.03151 |
| FOLH1 | NM_004476 | 1.41E−05 | 0.817179 | 0.968607 | −1.03241 |
| RGS2 | NM_002923 | 7.55E−06 | 0.000328 | 2.53448 | 2.53448 |
| NAT2 | NM_000015 | 4.42E−05 | 0.292778 | 1.22741 | 1.22741 |
| CCL25 | NM_005624 | 8.63E−05 | 0.690336 | 0.937517 | −1.06665 |
| SEMA6D | NM_153618 | 1.55E−05 | 0.945868 | 0.992365 | −1.00769 |
| ANXA13 | NM_001003954 | 2.27E−08 | 3.99E−07 | 11.2408 | 11.2408 |
| KLHL23 | ENST00000392647 | 8.54E−06 | 0.023326 | 1.52693 | 1.52693 |
| GSTA1 | NM_145740 | 1.25E−06 | 0.154535 | 0.693914 | −1.4411 |
| S100G | NM_004057 | 6.67E−05 | 0.166669 | 1.27986 | 1.27986 |
| LCT | NM_002299 | 1.06E−05 | 0.997468 | 1.00038 | 1.00038 |
| FAM5C | NM_199051 | 4.88E−06 | 0.38727 | 0.91647 | −1.09114 |
| ANPEP | NM_001150 | 3.32E−06 | 0.000306 | 2.31363 | 2.31363 |
| HIST1H2AE | NM_021052 | 0.001216 | 0.592642 | 0.885169 | −1.12973 |
| SLC11A2 | NM_000617 | 1.91E−06 | 0.192241 | 1.15331 | 1.15331 |
| LRRC19 | NM_022901 | 4.02E−06 | 0.009847 | 1.52777 | 1.52777 |
| SLC27A2 | NM_003645 | 1.60E−05 | 0.241477 | 1.265 | 1.265 |
| LDHC | NM_002301 | 5.51E−06 | 0.881698 | 0.985233 | −1.01499 |
| SCGN | NM_006998 | 0.000129 | 0.294288 | 0.845082 | −1.18332 |
| GPR160 | NM_014373 | 2.16E−05 | 0.934977 | 1.01721 | 1.01721 |
| SLC16A10 | NM_018593 | 0.000465 | 0.54731 | 1.12282 | 1.12282 |
| CLRN3 | NM_152311 | 4.69E−08 | 1.73E−06 | 6.49979 | 6.49979 |
| C12orf28 | BC143553 | 1.27E−05 | 0.000652 | 2.14142 | 2.14142 |
| SATB1 | NM_002971 | 0.000101 | 0.405523 | 1.26097 | 1.26097 |
| GOLT1A | NM_198447 | 4.68E−07 | 0.367544 | 1.08473 | 1.08473 |
| UFM1 | NM_016617 | 1.64E−05 | 0.381874 | 0.919883 | −1.08709 |
| HIBCH | NM_014362 | 0.011898 | 0.985899 | 0.995584 | −1.00444 |
| L1TD1 | NM_019079 | 0.000304 | 0.87407 | 0.956311 | −1.04568 |
| HOXA9 | NM_152739 | 2.96E−05 | 0.904783 | 1.0312 | 1.0312 |
| TPH1 | NM_004179 | 0.000951 | 0.843169 | 0.96601 | −1.03519 |
| HEPH | NM_138737 | 7.75E−08 | 5.35E−06 | 3.09377 | 3.09377 |
| BMS1P5 | NR_003611 | 0.240068 | 0.609905 | 1.37201 | 1.37201 |
| ASAH2 | NM_019893 | 7.62E−05 | 0.547895 | 1.08578 | 1.08578 |
| KIAA1324 | NM_020775 | 3.22E−08 | 2.29E−08 | 0.084891 | −11.7798 |
| ALDOC | NM_005165 | 2.49E−06 | 0.001135 | 1.58838 | 1.58838 |
| KPNA2 | NM_002266 | 0.022754 | 0.49921 | 1.24956 | 1.24956 |
| NEUROD1 | NM_002500 | 0.06316 | 0.67004 | 0.854231 | −1.17064 |
| MS4A8B | NM_031457 | 5.65E−06 | 0.538728 | 1.06533 | 1.06533 |
| EPHB2 | NM_017449 | 0.001129 | 0.354389 | 0.865019 | −1.15604 |
| MSI1 | NM_002442 | 9.22E−06 | 0.406005 | 1.09288 | 1.09288 |
| IFNK | NM_020124 | 0.002165 | 0.348535 | 1.47073 | 1.47073 |
| FGFBP1 | NM_005130 | 1.79E−08 | 2.67E−06 | 0.241632 | −4.13853 |
| CDKN1B | NM_004064 | 3.54E−05 | 0.080701 | 1.24482 | 1.24482 |
| TFPI | NM_006287 | 1.26E−05 | 0.119822 | 1.49285 | 1.49285 |
| STAMBPL1 | NM_020799 | 4.70E−06 | 0.902034 | 0.990904 | −1.00918 |
| NLGN4Y | NM_014893 | 4.39E−05 | 0.374066 | 1.20471 | 1.20471 |
| PLD1 | NM_002662 | 0.000446 | 0.042352 | 1.51016 | 1.51016 |
| APOBEC3B | NM_004900 | 0.001419 | 0.288154 | 1.24833 | 1.24833 |
| MEP1B | NM_005925 | 5.41E−05 | 0.61469 | 0.943529 | −1.05985 |
| | — | 0.001183 | 0.524315 | 0.817098 | −1.22384 |
| EPHX2 | NM_001979 | 1.10E−06 | 0.097773 | 1.12319 | 1.12319 |
| XRCC4 | NM_022550 | 0.001579 | 0.028941 | 2.25274 | 2.25274 |
| GAS2 | NM_005256 | 3.49E−05 | 0.391309 | 1.10823 | 1.10823 |
| DPP10 | NM_020868 | 0.000864 | 0.827068 | 0.965055 | −1.03621 |
| TLR4 | NR_024168 | 9.63E−05 | 0.960524 | 1.00726 | 1.00726 |
| LSAMP | NM_002338 | 2.16E−05 | 0.478213 | 0.918271 | −1.089 |
| SEPT7 | NM_001788 | 0.01691 | 0.535765 | 0.759353 | −1.31691 |
| CCNB2 | NM_004701 | 0.009939 | 0.652125 | 0.910009 | −1.09889 |
| MT1A | NM_005946 | 1.80E−05 | 2.09E−06 | 0.181046 | −5.52346 |
| C2orf43 | BC017473 | 0.002035 | 0.907738 | 1.0208 | 1.0208 |
| EML4 | NM_019063 | 0.003235 | 0.874733 | 1.03711 | 1.03711 |
| CKS2 | NM_001827 | 2.48E−05 | 0.253809 | 0.896221 | −1.1158 |
| CYP2B6 | NM_000767 | 0.000209 | 0.052391 | 1.5905 | 1.5905 |
| CCDC34 | NM_030771 | 4.73E−05 | 0.58764 | 1.05377 | 1.05377 |
| ADH6 | NM_001102470 | 2.18E−06 | 0.000522 | 2.005 | 2.005 |
| ATP8A1 | NM_006095 | 9.35E−06 | 0.919121 | 0.979759 | −1.02066 |
| FAR2 | NM_018099 | 3.78E−07 | 3.34E−05 | 1.80602 | 1.80602 |
| TF | NM_001063 | 7.43E−06 | 0.733031 | 0.974911 | −1.02574 |
| MYO1B | NM_001130158 | 1.47E−06 | 0.085899 | 1.37399 | 1.37399 |
| SLC35D1 | NM_015139 | 0.066551 | 0.848775 | 1.06074 | 1.06074 |
| CXorf52 | AY168775 | 0.026084 | 0.737231 | 1.17926 | 1.17926 |
| PCDH11Y | NM_032971 | 0.368856 | 0.94435 | 0.959851 | −1.04183 |
| SERPINE2 | NM_001136529 | 2.73E−07 | 0.798267 | 0.95069 | −1.05187 |
| ERP27 | NM_152321 | 0.002033 | 0.06942 | 1.50846 | 1.50846 |
| DNAJC2 | NM_014377 | 0.000601 | 0.248587 | 0.730929 | −1.36812 |
| PCDH20 | NM_022843 | 0.000951 | 0.938596 | 1.01243 | 1.01243 |
| HNF4G | NM_004133 | 3.36E−07 | 0.789701 | 1.04722 | 1.04722 |
| HIST1H3G | NM_003534 | 7.92E−05 | 0.642299 | 0.944271 | −1.05902 |
| HPDL | NM_032756 | 0.001394 | 0.85299 | 0.962647 | −1.0388 |
| SH3PXD2A | NM_014631 | 2.02E−05 | 0.003589 | 0.34797 | −2.87381 |
| COX18 | NM_173827 | 0.001081 | 0.986366 | 1.00279 | 1.00279 |
| HHLA2 | NM_007072 | 1.26E−05 | 0.062731 | 1.5504 | 1.5504 |
| ZNF770 | NM_014106 | 2.22E−05 | 0.36345 | 0.843946 | −1.18491 |
| LYPLA1 | NM_006330 | 5.75E−05 | 0.408349 | 0.815345 | −1.22648 |
| DHRS11 | NM_024308 | 0.000217 | 0.026583 | 1.54368 | 1.54368 |
| EPB41L2 | NM_001431 | 0.003371 | 0.243803 | 1.32387 | 1.32387 |
| EXOC3 | AK074086 | 1.49E−06 | 0.140389 | 1.11189 | 1.11189 |
| GHRL | NR_024138 | 0.027865 | 0.917204 | 0.974975 | −1.02567 |
| DACH1 | NM_080759 | 0.000217 | 0.930392 | 1.01044 | 1.01044 |
| SPARC | NM_003118 | 1.66E−06 | 0.745461 | 1.04966 | 1.04966 |
| SLCO4C1 | NM_180991 | 3.06E−05 | 0.128833 | 1.18801 | 1.18801 |
| KLHL23 | NM_144711 | 0.000249 | 0.113175 | 1.40747 | 1.40747 |
| KRT6B | NM_005555 | 9.83E−11 | 0.091889 | 0.85651 | −1.16753 |
| EPCAM | NM_002354 | 1.17E−07 | 0.395177 | 1.13504 | 1.13504 |
| IL20RB | NM_144717 | 7.88E−07 | 0.782018 | 0.934046 | −1.07061 |
| MEIS2 | NM_172316 | 5.41E−06 | 0.001467 | 1.50829 | 1.50829 |
| MMP12 | NM_002426 | 0.003373 | 0.45577 | 1.14604 | 1.14604 |
| ACPL2 | NM_152282 | 8.11E−06 | 0.30308 | 1.09 | 1.09 |
| TIMP3 | NM_000362 | 3.21E−07 | 0.325432 | 0.878479 | −1.13833 |
| CXCL14 | NM_004887 | 0.000211 | 0.329507 | 1.25497 | 1.25497 |
| METTL6 | NM_152396 | 0.001275 | 0.389509 | 0.809846 | −1.2348 |
| ZNF770 | NM_014106 | 1.21E−06 | 0.825134 | 0.979436 | −1.021 |
| CLDND1 | NM_001040199 | 0.000346 | 0.168171 | 0.76108 | −1.31392 |
| RAET1L | NM_130900 | 5.71E−06 | 0.000821 | 0.283356 | −3.52913 |
| SDAD1 | NM_018115 | 0.022444 | 0.149688 | 0.593853 | −1.68392 |
| PLEKHF2 | NM_024613 | 0.005965 | 0.648635 | 0.853017 | −1.17231 |
| TMEM117 | NM_032256 | 0.000172 | 0.139495 | 0.732889 | −1.36446 |
| RASA1 | NM_002890 | 0.000185 | 0.674089 | 0.924542 | −1.08162 |
| S100A16 | NM_080388 | 2.27E−05 | 0.117999 | 0.756328 | −1.32218 |
| KCTD9 | NM_017634 | 0.000344 | 0.058892 | 0.655909 | −1.5246 |
| GRHL1 | NM_014552 | 2.68E−07 | 0.670364 | 1.05302 | 1.05302 |
| ARHGAP29 | NM_004815 | 8.76E−05 | 0.827322 | 1.05553 | 1.05553 |
| BNIP2 | NM_004330 | 4.25E−05 | 0.276324 | 1.24493 | 1.24493 |
| MARCH7 | NM_022826 | 0.017224 | 0.205183 | 0.662246 | −1.51001 |
| RAB23 | NM_016277 | 0.001104 | 0.884425 | 1.03235 | 1.03235 |
| STK17A | NM_004760 | 0.001954 | 0.683331 | 0.862241 | −1.15977 |
| REEP3 | ENST00000298249 | 0.000142 | 0.740284 | 0.932601 | −1.07227 |
| ATL2 | NM_022374 | 0.002578 | 0.213772 | 0.722131 | −1.38479 |
| MALT1 | NM_006785 | 3.81E−07 | 0.056831 | 0.829054 | −1.20619 |
| LOC554203 | NR_024582 | 0.005588 | 0.849364 | 1.06021 | 1.06021 |
| DUSP11 | NM_003584 | 8.27E−05 | 0.380791 | 0.85819 | −1.16524 |
| IGF2BP2 | NM_006548 | 0.00154 | 0.276382 | 1.32037 | 1.32037 |
| SEPT10 | NM_144710 | 0.005078 | 0.295213 | 0.754419 | −1.32552 |
| REPS1 | NM_031922 | 0.001111 | 0.130513 | 0.716519 | −1.39564 |
| C3orf14 | AF236158 | 0.000139 | 0.036881 | 1.883 | 1.883 |
| ADK | NM_006721 | 6.58E−05 | 0.918164 | 1.01505 | 1.01505 |
| SSR3 | NM_007107 | 0.010975 | 0.187956 | 0.655579 | −1.52537 |
| PRRG4 | NM_024081 | 3.02E−05 | 0.040066 | 0.74722 | −1.33829 |
| PDPN | NM_006474 | 8.09E−07 | 0.908853 | 1.01679 | 1.01679 |
| KIAA1586 | NM_020931 | 1.65E−05 | 0.815177 | 1.0374 | 1.0374 |
| PEX3 | NM_003630 | 1.98E−05 | 0.82392 | 1.04112 | 1.04112 |
| | — | 0.000761 | 0.393051 | 1.21631 | 1.21631 |
| EIF2AK2 | NM_002759 | 0.012021 | 0.374086 | 0.751052 | −1.33147 |
| GTF2F2 | NM_004128 | 0.000579 | 0.377913 | 0.856518 | −1.16752 |
| SMYD2 | NM_020197 | 7.87E−05 | 0.407177 | 0.898136 | −1.11342 |
| CTSC | NM_001814 | 1.37E−07 | 0.278963 | 0.892579 | −1.12035 |
| MPP7 | NM_173496 | 1.95E−07 | 0.000139 | 1.79904 | 1.79904 |
| GDAP1 | NM_018972 | 1.52E−06 | 0.00234 | 1.54283 | 1.54283 |
| FN1 | NM_212482 | 0.000112 | 0.117263 | 1.55772 | 1.55772 |
| TROVE2 | NM_004600 | 0.004196 | 0.795748 | 0.940558 | −1.0632 |
| C1orf149 | NM_022756 | 0.000508 | 0.277797 | 0.836815 | −1.19501 |
| CLEC2B | NM_005127 | 0.003209 | 0.86596 | 0.945449 | −1.0577 |
| ALS2CR4 | NM_001044385 | 1.40E−06 | 0.189331 | 1.14579 | 1.14579 |
| PTPN12 | NM_002835 | 0.001268 | 0.344662 | 1.26374 | 1.26374 |
| BOD1L | NM_148894 | 0.007872 | 0.484484 | 0.83063 | −1.20391 |
| TNNT1 | NM_003283 | 3.38E−06 | 0.23944 | 1.18015 | 1.18015 |
| FABP7 | NM_001446 | 0.012746 | 0.674857 | 0.895753 | −1.11638 |
| HDGFRP3 | NM_016073 | 5.09E−07 | 0.009735 | 1.35995 | 1.35995 |
| SPRR2D | NM_006945 | 1.59E−06 | 0.384946 | 1.35186 | 1.35186 |
| FJX1 | NM_014344 | 4.64E−06 | 0.973508 | 1.00484 | 1.00484 |
| S100A14 | NM_020672 | 9.03E−05 | 0.19977 | 0.749712 | −1.33385 |
| MT1M | NM_176870 | 6.30E−07 | 7.31E−07 | 0.114584 | −8.72719 |
| LRRC37B2 | NR_015341 | 0.000454 | 0.741343 | 0.94108 | −1.06261 |
| IL18 | NM_001562 | 2.33E−06 | 0.261551 | 1.20103 | 1.20103 |
| GABRE | NM_004961 | 5.51E−05 | 0.348973 | 1.13527 | 1.13527 |
| GNPDA2 | NM_138335 | 5.96E−05 | 0.419588 | 1.11695 | 1.11695 |
| ELOVL4 | NM_022726 | 2.26E−07 | 0.904397 | 1.02091 | 1.02091 |
| WASF1 | NM_003931 | 4.90E−05 | 0.254721 | 1.24822 | 1.24822 |
| PIK3CA | NM_006218 | 0.000544 | 0.862504 | 1.03449 | 1.03449 |
| MBOAT2 | NM_138799 | 0.000192 | 0.004061 | 0.34441 | −2.90351 |
| PAR1 | AF019616 | 0.000965 | 0.300889 | 1.24773 | 1.24773 |
| IVNS1ABP | NM_006469 | 0.006048 | 0.335337 | 0.80216 | −1.24663 |
| CHIC2 | NM_012110 | 0.000122 | 0.140178 | 0.798603 | −1.25219 |
| VSNL1 | NM_003385 | 3.02E−08 | 3.48E−05 | 2.75331 | 2.75331 |
| LRRC37A3 | NM_199340 | 0.00638 | 0.446635 | 0.845344 | −1.18295 |
| FYTTD1 | NM_001011537 | 0.004033 | 0.498991 | 0.861433 | −1.16086 |
| RNF217 | NM_152553 | 1.89E−10 | 0.000313 | 1.38293 | 1.38293 |
| PLA2G4A | NM_024420 | 0.006562 | 0.304921 | 0.775583 | −1.28935 |
| P2RY5 | NM_005767 | 2.90E−06 | 0.68288 | 1.05684 | 1.05684 |
| NT5E | NM_002526 | 2.00E−07 | 0.114638 | 0.786981 | −1.27068 |
| CTSL2 | NM_001333 | 1.96E−05 | 0.629843 | 0.908449 | −1.10078 |
| ZNF354A | NM_005649 | 0.006993 | 0.240775 | 0.767069 | −1.30366 |
| KIFAP3 | NM_014970 | 2.61E−06 | 0.06349 | 1.30939 | 1.30939 |
| RAB18 | NM_021252 | 4.91E−05 | 0.049838 | 0.730702 | −1.36855 |
| C1orf74 | BC039719 | 7.17E−05 | 0.688993 | 1.05848 | 1.05848 |
| RB1 | NM_000321 | 0.000478 | 0.076805 | 0.735413 | −1.35978 |
| CEP170 | NM_014812 | 3.10E−05 | 0.067147 | 1.55308 | 1.55308 |
| KIF13A | NM_022113 | 7.87E−06 | 0.933653 | 1.01045 | 1.01045 |
| PRKCQ | NM_006257 | 5.36E−06 | 0.942759 | 0.992509 | −1.00755 |
| C6orf105 | NM_001143948 | 9.93E−05 | 0.007267 | 0.51191 | −1.95347 |
| KRT23 | NM_015515 | 5.21E−08 | 0.00182 | 1.59222 | 1.59222 |
| C10orf55 | NM_001001791 | 0.004044 | 0.453532 | 0.815553 | −1.22616 |
| EFTUD1 | NM_024580 | 5.31E−05 | 0.855198 | 0.977134 | −1.0234 |
| EDNRA | NM_001957 | 0.00118 | 0.939445 | 0.984322 | −1.01593 |
| TMTC1 | NM_175861 | 8.69E−08 | 0.211894 | 0.882779 | −1.13279 |
| DUSP14 | NM_007026 | 3.77E−06 | 0.3473 | 1.11611 | 1.11611 |
| GPNMB | NM_001005340 | 1.01E−06 | 0.850774 | 1.03534 | 1.03534 |
| PRSS3 | NM_007343 | 0.001276 | 0.384432 | 0.84951 | −1.17715 |
| EMB | NM_198449 | 2.20E−07 | 0.075382 | 1.21044 | 1.21044 |
| SLC1A3 | NM_004172 | 3.98E−07 | 2.90E−05 | 0.475033 | −2.10512 |
| TCTEX1D2 | NM_152773 | 5.43E−08 | 0.347454 | 1.05358 | 1.05358 |
| NUDT11 | NM_018159 | 0.000877 | 0.980617 | 0.991066 | −1.00901 |
| AIG1 | NM_016108 | 4.81E−05 | 0.104716 | 1.26639 | 1.26639 |
| NEDD4 | NM_006154 | 6.37E−05 | 0.261659 | 0.871193 | −1.14785 |
| MMP10 | NM_002425 | 0.005493 | 0.737511 | 0.846256 | −1.18167 |
| NDFIP2 | NM_019080 | 9.17E−05 | 0.664582 | 1.06969 | 1.06969 |
| D4S234E | NM_014392 | 2.06E−05 | 0.917355 | 1.02137 | 1.02137 |
| PCTK2 | NM_002595 | 8.48E−06 | 0.424519 | 1.09366 | 1.09366 |
| KIAA0922 | NM_001131007 | 5.88E−07 | 0.692272 | 0.958666 | −1.04312 |
| EFCAB2 | NR_026588 | 0.023377 | 0.665681 | 0.872544 | −1.14607 |
| RABGEF1 | NM_014504 | 0.002164 | 0.389878 | 0.851125 | −1.17492 |
| MCART1 | NR_024873 | 0.056152 | 0.185878 | 0.538811 | −1.85594 |
| IGFL3 | NM_207393 | 9.80E−08 | 0.036733 | 0.848766 | −1.17818 |
| ANTXR2 | NM_058172 | 5.41E−06 | 0.801006 | 0.969992 | −1.03094 |
| FBN2 | NM_001999 | 2.36E−07 | 0.532557 | 1.08876 | 1.08876 |
| SCFD1 | NM_016106 | 0.008561 | 0.269726 | 0.769348 | −1.2998 |
| C11orf60 | NM_020153 | 2.60E−06 | 0.032384 | 0.802562 | −1.24601 |
| UNQ1887 | NM_139015 | 6.66E−07 | 0.010253 | 1.24687 | 1.24687 |
| HOMER1 | NM_004272 | 0.001456 | 0.563836 | 0.870836 | −1.14832 |
| LPAR3 | NM_012152 | 1.27E−07 | 0.819446 | 0.974058 | −1.02663 |
| LRRC42 | NM_052940 | 0.000315 | 0.553792 | 0.922073 | −1.08451 |
| GOLGA8B | NR_027410 | 3.19E−05 | 0.000483 | 2.86234 | 2.86234 |
| CYB5R2 | NM_016229 | 0.000242 | 0.033137 | 0.635253 | −1.57418 |
| UBE2F | NM_080678 | 0.0049 | 0.324724 | 0.809307 | −1.23562 |
| TMTC3 | NM_181783 | 2.84E−05 | 0.718184 | 0.958463 | −1.04334 |
| ZCCHC11 | NM_001009881 | 0.000345 | 0.259485 | 1.26918 | 1.26918 |
| PPP3CC | NM_005605 | 0.000139 | 0.40974 | 0.879464 | −1.13706 |
| SESN3 | NM_144665 | 1.87E−05 | 0.644605 | 0.938198 | −1.06587 |
| C14orf149 | NM_144581 | 5.47E−05 | 0.365414 | 0.899314 | −1.11196 |
| PTPLA | NM_014241 | 9.50E−07 | 0.419147 | 0.937001 | −1.06723 |
| ODF2L | NM_020729 | 5.20E−05 | 0.01168 | 1.61203 | 1.61203 |
| FAM174A | NM_198507 | 0.001283 | 0.049476 | 0.608776 | −1.64264 |
| CBL | NM_005188 | 4.47E−06 | 0.736706 | 0.973469 | −1.02725 |
| PDCD1LG2 | NM_025239 | 0.000571 | 0.603517 | 0.892033 | −1.12103 |
| PMAIP1 | NM_021127 | 1.14E−05 | 0.002687 | 1.81509 | 1.81509 |
| SACS | NM_014363 | 9.98E−06 | 0.02187 | 1.56338 | 1.56338 |
| FKBP14 | NM_017946 | 0.000421 | 0.78068 | 0.952519 | −1.04985 |
| ROBO1 | NM_133631 | 5.83E−07 | 0.960284 | 1.00768 | 1.00768 |
| QPCT | NM_012413 | 0.000184 | 0.486296 | 1.13839 | 1.13839 |
| ZFP42 | NM_174900 | 0.048358 | 0.907614 | 1.04601 | 1.04601 |
| DSP | NM_004415 | 2.60E−05 | 0.777212 | 0.962674 | −1.03877 |
| SPRR1A | NM_005987 | 1.72E−08 | 0.000821 | 0.464088 | −2.15476 |
| IL1A | NM_000575 | 8.93E−10 | 0.025527 | 0.7224 | −1.38428 |
| LOC654433 | NR_015377 | 0.146494 | 0.503143 | 0.728255 | −1.37315 |
| EPS15 | NM_001981 | 0.013543 | 0.49308 | 0.85887 | −1.16432 |
| S100A11 | NM_005620 | 0.000261 | 0.315911 | 0.855889 | −1.16838 |
| SLC36A4 | NM_152313 | 3.46E−05 | 0.011347 | 0.602895 | −1.65866 |
| RRAGC | NM_022157 | 0.000313 | 0.423692 | 0.904501 | −1.10558 |
| DOCK11 | NM_144658 | 8.05E−07 | 0.035947 | 0.717119 | −1.39447 |
| KDSR | NM_002035 | 5.66E−08 | 0.028676 | 1.16883 | 1.16883 |
| ERGIC2 | NM_016570 | 0.000598 | 0.595637 | 0.922475 | −1.08404 |
| CSGALNACT2 | NM_018590 | 0.000202 | 0.509818 | 1.09982 | 1.09982 |
| LOC554202 | NR_027054 | 1.47E−07 | 0.056703 | 1.22206 | 1.22206 |
| WFDC5 | NM_145652 | 7.11E−06 | 0.50016 | 0.878851 | −1.13785 |
| PLXDC2 | NM_032812 | 1.91E−08 | 0.777947 | 0.970972 | −1.0299 |
| FBXW7 | NM_033632 | 0.001172 | 0.587438 | 0.911701 | −1.09685 |
| TMEM69 | NM_016486 | 0.000973 | 0.328165 | 1.1862 | 1.1862 |
| TMEM45A | NM_018004 | 1.87E−11 | 0.057522 | 0.845463 | −1.18278 |
| BBS10 | NM_024685 | 0.001013 | 0.986878 | 0.997303 | −1.0027 |
| SOX2OT | NR_004053 | 0.003922 | 0.001297 | 0.252645 | −3.95812 |
| KDM5B | NM_006618 | 0.00124 | 0.8956 | 0.979129 | −1.02132 |
| CDA | NM_001785 | 7.22E−05 | 0.853675 | 1.04127 | 1.04127 |
| IFIT5 | NM_012420 | 0.001922 | 0.985909 | 0.99644 | −1.00357 |
| GTF2H1 | NM_001142307 | 0.000615 | 0.471872 | 0.899256 | −1.11203 |
| NEFM | NM_005382 | 0.000894 | 0.435712 | 1.27359 | 1.27359 |
| SGCE | NM_001099401 | 3.31E−05 | 0.00329 | 1.68958 | 1.68958 |
| DIRC2 | NM_032839 | 3.81E−05 | 0.00751 | 1.51859 | 1.51859 |
| ITGA1 | NM_181501 | 3.45E−05 | 0.254011 | 1.12878 | 1.12878 |
| RSAD2 | NM_080657 | 0.014966 | 0.706236 | 1.15174 | 1.15174 |
| SLFN5 | NM_144975 | 2.52E−05 | 0.000355 | 3.21394 | 3.21394 |
| SLC2A3 | NM_006931 | 1.19E−05 | 0.971294 | 0.992216 | −1.00784 |
| ADAMTS1 | NM_006988 | 8.15E−06 | 0.091202 | 1.24299 | 1.24299 |
| ZBTB1 | NM_001123329 | 0.00013 | 0.407351 | 0.914589 | −1.09339 |
| PIP5K1A | NM_001135638 | 0.026998 | 0.589881 | 0.852287 | −1.17331 |
| DFNA5 | NM_004403 | 2.70E−06 | 0.005366 | 1.72407 | 1.72407 |
| DMKN | NM_033317 | 4.50E−07 | 0.141932 | 1.16214 | 1.16214 |
| FLRT3 | NM_198391 | 7.17E−07 | 0.000218 | 2.58604 | 2.58604 |
| SPRR3 | NM_005416 | 1.85E−07 | 0.490821 | 0.898669 | −1.11276 |
| TTPAL | NM_024331 | 2.86E−06 | 0.868857 | 1.01788 | 1.01788 |
| RPS6KA5 | NM_004755 | 4.57E−05 | 0.967021 | 0.996061 | −1.00395 |
| CLN5 | NM_006493 | 0.000897 | 0.458264 | 0.902943 | −1.10749 |
| EFEMP1 | NM_004105 | 2.65E−08 | 0.342209 | 0.86779 | −1.15235 |
| SLC20A1 | NM_005415 | 6.24E−06 | 0.559718 | 0.919276 | −1.08781 |
| GNAI1 | NM_002069 | 0.000112 | 0.004161 | 2.32485 | 2.32485 |
| FERMT1 | NM_017671 | 0.000364 | 0.51844 | 0.918142 | −1.08916 |
| FN1 | NM_212482 | 9.48E−07 | 0.344093 | 1.25565 | 1.25565 |
| GJB6 | NM_001110219 | 1.86E−06 | 0.280012 | 1.33981 | 1.33981 |
| GPR1 | NM_005279 | 6.76E−05 | 0.920185 | 0.989886 | −1.01022 |
| GPR115 | NM_153838 | 1.43E−05 | 0.875384 | 0.972789 | −1.02797 |
| ZNF607 | NM_032689 | 4.95E−06 | 0.742897 | 0.975057 | −1.02558 |
| MTHFD2L | NM_001144978 | 2.12E−06 | 0.857342 | 1.01476 | 1.01476 |
| LRAT | NM_004744 | 1.74E−05 | 0.802563 | 1.04117 | 1.04117 |
| C3orf64 | NM_173654 | 0.000232 | 0.065142 | 0.707731 | −1.41297 |
| ALDH3B2 | NM_000695 | 0.000138 | 0.226623 | 1.17486 | 1.17486 |
| MT1X | NM_005952 | 0.002024 | 0.813716 | 1.06879 | 1.06879 |
| USP25 | NM_013396 | 0.000737 | 0.269933 | 0.853343 | −1.17186 |
| USP53 | NM_019050 | 0.007056 | 0.936263 | 1.01602 | 1.01602 |
| DUSP6 | NM_001946 | 0.00301 | 0.639665 | 1.09556 | 1.09556 |
| TLE4 | NM_007005 | 5.00E−05 | 0.008608 | 1.53717 | 1.53717 |
| INHBA | NM_002192 | 0.000639 | 0.631387 | 0.905254 | −1.10466 |
| COL12A1 | NM_004370 | 3.22E−07 | 0.409874 | 1.13641 | 1.13641 |
| SLIT2 | NM_004787 | 8.48E−06 | 0.680656 | 0.957377 | −1.04452 |
| KLF8 | NM_007250 | 1.93E−05 | 0.394299 | 0.902273 | −1.10831 |
| IQCA1 | NM_024726 | 3.76E−08 | 0.315482 | 0.89791 | −1.1137 |
| BNC1 | NM_001717 | 2.55E−08 | 0.37964 | 0.906132 | −1.10359 |
| TCFL5 | NM_006602 | 4.73E−05 | 0.07954 | 1.23475 | 1.23475 |
| S100A7 | NM_002963 | 8.46E−06 | 0.764639 | 1.12581 | 1.12581 |
| EMP3 | NM_001425 | 0.000135 | 0.782887 | 1.07599 | 1.07599 |
| DEGS1 | NM_003676 | 0.000529 | 0.364489 | 1.34899 | 1.34899 |
| SPG20 | NM_001142295 | 0.000483 | 0.741705 | 1.05335 | 1.05335 |
| TPD52L1 | NM_001003395 | 1.12E−05 | 0.787244 | 1.02975 | 1.02975 |
| GPR137B | NM_003272 | 1.43E−05 | 0.891138 | 0.984364 | −1.01588 |
| NIACR2 | NM_006018 | 2.84E−05 | 0.685785 | 1.08487 | 1.08487 |
| RBMS3 | NM_001003793 | 0.000283 | 0.732447 | 0.958713 | −1.04307 |
| MUC15 | NM_001135091 | 2.23E−07 | 0.423441 | 0.877041 | −1.1402 |
| PPP4R1 | NM_001042388 | 6.81E−05 | 0.325871 | 0.890068 | −1.12351 |
| FCHO2 | NM_138782 | 0.041643 | 0.577163 | 0.831338 | −1.20288 |
| LEF1 | NM_016269 | 0.01014 | 0.975765 | 0.992718 | −1.00733 |
| CLASP 1 | NM_015282 | 0.000646 | 0.506131 | 0.900357 | −1.11067 |
| TMEM154 | NM_152680 | 3.06E−07 | 0.906144 | 1.01745 | 1.01745 |
| IKIP | NM_153687 | 2.32E−08 | 0.02648 | 0.77252 | −1.29446 |
| HIVEP2 | NM_006734 | 4.23E−07 | 0.001835 | 1.68848 | 1.68848 |
| DSC3 | NM_024423 | 1.04E−10 | 0.148326 | 0.85442 | −1.17039 |
| CLDN1 | NM_021101 | 1.30E−06 | 9.63E−05 | 2.02073 | 2.02073 |
| GJB2 | NM_004004 | 3.20E−09 | 0.511514 | 1.05777 | 1.05777 |
| WDR47 | NM_001142550 | 5.48E−05 | 0.430766 | 0.867016 | −1.15338 |
| SPINK5 | NM_001127698 | 4.21E−06 | 0.019621 | 0.527906 | −1.89428 |
| S1PR1 | NM_001400 | 2.02E−06 | 0.479086 | 0.913656 | −1.0945 |
| IL1RAP | NM_002182 | 2.28E−07 | 0.468287 | 1.08077 | 1.08077 |
| VEGFC | NM_005429 | 2.82E−05 | 0.731719 | 1.05089 | 1.05089 |
| AHSA2 | NM_152392 | 0.007404 | 0.225778 | 1.40222 | 1.40222 |
| FBXO3 | NM_033406 | 8.51E−05 | 0.807121 | 0.972252 | −1.02854 |
| SRY | NM_003140 | 0.000306 | 0.385178 | 0.837197 | −1.19446 |
| RPSAP52 | NR_026825 | 5.72E−06 | 0.451358 | 1.09257 | 1.09257 |
| TAGLN3 | NM_013259 | 7.20E−06 | 0.515786 | 1.08487 | 1.08487 |
| BACH1 | NM_206866 | 3.25E−07 | 0.070202 | 0.901991 | −1.10866 |
| LY6G6C | NM_025261 | 0.000167 | 0.516811 | 0.911763 | −1.09678 |
| ARL17P1 | NM_001113738 | 0.065934 | 0.896144 | 1.04782 | 1.04782 |
| PTGS1 | NM_000962 | 0.000309 | 0.92096 | 0.983269 | −1.01702 |
| NRG1 | NM_013960 | 1.99E−06 | 0.700954 | 0.955373 | −1.04671 |
| TCF4 | NM_001083962 | 1.44E−05 | 0.465419 | 1.06152 | 1.06152 |
| ZFYVE9 | NM_004799 | 0.00013 | 0.933287 | 1.01076 | 1.01076 |
| FAM83A | NM_032899 | 3.35E−06 | 0.237368 | 0.883703 | −1.1316 |
| ITGA2 | NM_002203 | 0.000116 | 0.559486 | 0.919839 | −1.08715 |
| HERC6 | NM_017912 | 0.000901 | 0.954878 | 0.984915 | −1.01532 |
| FHL1 | NM_001159704 | 2.69E−05 | 0.266689 | 1.1672 | 1.1672 |
| USP9Y | NM_004654 | 2.78E−05 | 0.066875 | 1.30466 | 1.30466 |
| PLAU | NM_002658 | 0.000318 | 0.2318 | 1.23969 | 1.23969 |
| FGF11 | NM_004112 | 0.000923 | 0.559071 | 0.922811 | −1.08365 |
| CYP4F12 | NM_023944 | 2.48E−06 | 0.000355 | 0.457181 | −2.18732 |
| BCAT1 | NM_005504 | 2.88E−05 | 0.640106 | 1.08261 | 1.08261 |
| KLK8 | NM_144505 | 3.28E−08 | 0.948291 | 0.992733 | −1.00732 |
| BPIL2 | NM_174932 | 3.22E−07 | 0.324773 | 0.907337 | −1.10213 |
| GLI3 | NM_000168 | 1.10E−05 | 0.70959 | 0.950737 | −1.05182 |
| ZBED2 | NM_024508 | 1.18E−06 | 0.533492 | 0.882086 | −1.13368 |
| AADACL2 | NM_207365 | 8.51E−05 | 0.878873 | 1.02832 | 1.02832 |
| RHCG | NM_016321 | 0.001734 | 0.897862 | 1.04188 | 1.04188 |
| CCNA1 | NM_003914 | 3.86E−06 | 0.551723 | 1.12086 | 1.12086 |
| CA12 | NM_001218 | 8.75E−06 | 0.001318 | 0.578875 | −1.72749 |
| S100A12 | NM_005621 | 5.41E−05 | 0.62284 | 1.11988 | 1.11988 |
| TP53AIP1 | NM_022112 | 2.01E−06 | 0.780029 | 0.971965 | −1.02884 |
| IFNA1 | NM_024013 | 0.00099 | 0.576181 | 1.10418 | 1.10418 |
| DENND2C | NM_198459 | 1.87E−08 | 0.080943 | 0.897954 | −1.11364 |
| DSE | NM_013352 | 2.20E−07 | 0.073832 | 1.36852 | 1.36852 |
| SLC26A2 | NM_000112 | 0.000313 | 0.721084 | 0.95225 | −1.05014 |
| RECQL | NM_002907 | 0.022406 | 0.952671 | 1.01346 | 1.01346 |
| SERPINB4 | NM_002974 | 1.08E−05 | 0.69221 | 0.893295 | −1.11945 |
| UPP1 | NM_003364 | 5.20E−06 | 0.169556 | 1.12327 | 1.12327 |
| PTER | NM_030664 | 2.00E−06 | 1.74E−05 | 0.316463 | −3.15992 |
| IVL | NM_005547 | 1.03E−07 | 0.187625 | 0.769062 | −1.30028 |
| GJC1 | NM_005497 | 5.99E−05 | 0.000343 | 2.89407 | 2.89407 |
| SLC2A1 | NM_006516 | 1.53E−07 | 0.104053 | 0.876208 | −1.14128 |
| SLC10A6 | NM_197965 | 2.31E−06 | 0.62639 | 1.06766 | 1.06766 |
| CLIP1 | NM_002956 | 1.41E−05 | 0.254683 | 0.879449 | −1.13708 |
| TPM2 | NM_003289 | 3.73E−05 | 0.040905 | 1.51262 | 1.51262 |
| CNTN1 | NM_001843 | 2.39E−09 | 0.517092 | 1.07211 | 1.07211 |
| SLC7A5 | NM_003486 | 1.38E−05 | 0.00079 | 1.83556 | 1.83556 |
| PAQR7 | NM_178422 | 0.000521 | 0.065449 | 1.42775 | 1.42775 |
| FBLN1 | NM_006486 | 0.000326 | 0.980281 | 0.997089 | −1.00292 |
| SEMA3D | NM_152754 | 0.002343 | 0.261945 | 0.691205 | −1.44675 |
| CCDC3 | NM_031455 | 0.000571 | 0.712647 | 1.08856 | 1.08856 |
| TRAF3IP3 | NM_025228 | 0.000398 | 0.973547 | 1.00592 | 1.00592 |
| NETO1 | NM_138966 | 0.10033 | 0.961758 | 1.01528 | 1.01528 |
| BCO2 | NM_031938 | 6.33E−06 | 0.606651 | 1.07471 | 1.07471 |
| AMIGO2 | NM_001143668 | 4.45E−08 | 0.053988 | 1.3033 | 1.3033 |
| KRT4 | NM_002272 | 6.63E−07 | 0.884442 | 0.975147 | −1.02549 |
| AKTIP | NM_001012398 | 0.006513 | 0.332753 | 1.27583 | 1.27583 |
| SP100 | NM_001080391 | 0.000455 | 0.710058 | 1.08744 | 1.08744 |
| THSD1P | NR_002816 | 0.002072 | 0.423221 | 1.14732 | 1.14732 |
| TMEM136 | NM_174926 | 8.25E−06 | 0.501499 | 0.929729 | −1.07558 |
| TTLL7 | NM_024686 | 0.000107 | 0.525888 | 0.904002 | −1.10619 |
| RND3 | NM_005168 | 7.33E−05 | 0.588714 | 1.06102 | 1.06102 |
| TACSTD2 | NM_002353 | 1.62E−06 | 0.851125 | 0.988506 | −1.01163 |
| RBP7 | NM_052960 | 2.56E−05 | 0.351309 | 1.23374 | 1.23374 |
| OR10A3 | NM_001003745 | 3.64E−05 | 0.219649 | 0.809049 | −1.23602 |
| PLA2R1 | NM_007366 | 2.45E−07 | 0.008386 | 1.49126 | 1.49126 |
| KRTDAP | NM_207392 | 1.02E−08 | 0.321868 | 1.20972 | 1.20972 |
| PRNP | NM_000311 | 2.18E−06 | 0.640739 | 1.05036 | 1.05036 |
| SLC9A9 | NM_173653 | 0.000842 | 0.639565 | 1.07675 | 1.07675 |
| CDC42SE1 | NM_001038707 | 1.84E−05 | 0.837519 | 1.02216 | 1.02216 |
| KLK5 | NM_012427 | 9.59E−07 | 0.750788 | 0.940019 | −1.06381 |
| KTN1 | NM_182926 | 0.001011 | 0.939355 | 1.01093 | 1.01093 |
| KRT1 | NM_006121 | 6.75E−07 | 0.670907 | 0.919253 | −1.08784 |
| RGS20 | NM_170587 | 6.81E−05 | 0.132416 | 1.22492 | 1.22492 |
| LHFP | NM_005780 | 9.78E−05 | 0.948746 | 0.990977 | −1.00911 |
| C21orf91 | NM_001100420 | 3.26E−05 | 0.022371 | 0.616613 | −1.62176 |
| ST3GAL5 | NM_003896 | 1.62E−05 | 0.616866 | 0.925493 | −1.08051 |
| KRT24 | NM_019016 | 8.72E−06 | 0.981304 | 1.0031 | 1.0031 |
| DSG1 | NM_001942 | 1.97E−11 | 0.720834 | 1.03361 | 1.03361 |
| PLAT | NM_000930 | 0.001276 | 0.899146 | 1.01802 | 1.01802 |
| THBS2 | NM_003247 | 4.78E−07 | 0.736915 | 1.04266 | 1.04266 |
| NIACR1 | NM_177551 | 1.23E−05 | 0.889664 | 1.02556 | 1.02556 |
| DSC1 | NM_004948 | 1.38E−08 | 0.969036 | 1.00566 | 1.00566 |
| AQP9 | NM_020980 | 0.001541 | 0.630912 | 0.902674 | −1.10782 |
| BNIPL | NM_001159642 | 5.78E−06 | 0.885151 | 1.01605 | 1.01605 |
| TNFAIP3 | NM_006290 | 9.56E−05 | 0.559051 | 1.05827 | 1.05827 |
| LASS3 | NM_178842 | 3.98E−09 | 0.314531 | 1.11074 | 1.11074 |
| RUFY2 | NM_017987 | 3.06E−05 | 0.429326 | 1.0966 | 1.0966 |
| SLC26A9 | NM_052934 | 5.61E−07 | 1.24E−06 | 0.243313 | −4.10993 |
| RORA | NM_134260 | 0.0006 | 0.512618 | 0.916029 | −1.09167 |
| AMOTL1 | NM_130847 | 2.34E−07 | 0.024114 | 1.29608 | 1.29608 |
| CARD18 | NM_021571 | 1.65E−06 | 0.866603 | 1.03026 | 1.03026 |
| C20orf197 | NM_173644 | 0.012273 | 0.852902 | 1.04266 | 1.04266 |
| CAPN6 | NM_014289 | 1.50E−06 | 3.49E−06 | 0.197617 | −5.06028 |
| TUBB6 | NM_032525 | 2.26E−06 | 0.001073 | 1.62731 | 1.62731 |
| CCDC80 | NM_199511 | 5.69E−06 | 0.952031 | 1.00945 | 1.00945 |
| TEX2 | NM_018469 | 1.14E−06 | 0.063031 | 0.872803 | −1.14573 |
| EEA1 | NM_003566 | 0.000621 | 0.286705 | 0.842252 | −1.18729 |
| RAET1G | NM_001001788 | 7.86E−06 | 0.839722 | 1.04317 | 1.04317 |
| NR3C1 | NM_000176 | 3.04E−05 | 0.940111 | 0.992877 | −1.00717 |
| NCF2 | NM_000433 | 4.35E−06 | 0.972574 | 0.996317 | −1.0037 |
| TRIML2 | NM_173553 | 0.035133 | 0.791454 | 1.0847 | 1.0847 |
| SLC31A2 | NM_001860 | 7.56E−07 | 0.146444 | 0.84624 | −1.1817 |
| ANO4 | NM_178826 | 0.137128 | 0.998072 | 1.00091 | 1.00091 |
| SBSN | NM_198538 | 1.23E−09 | 0.588773 | 1.06842 | 1.06842 |
| ELAVL2 | NM_004432 | 4.22E−06 | 0.923808 | 0.99077 | −1.00932 |
| BIVM | NM_017693 | 0.000217 | 0.701966 | 1.04755 | 1.04755 |
| LAMC2 | NM_005562 | 1.16E−06 | 0.968733 | 1.00497 | 1.00497 |
| PHLDB2 | NM_001134438 | 9.26E−06 | 0.635598 | 1.07359 | 1.07359 |
| SFRS12IP1 | NM_173829 | 0.001825 | 0.694847 | 1.07242 | 1.07242 |
| SYT14 | NM_001146261 | 1.09E−07 | 0.451118 | 1.11055 | 1.11055 |
| DGKH | NM_178009 | 6.27E−06 | 0.520206 | 1.05387 | 1.05387 |
| KRT10 | NM_000421 | 1.21E−09 | 0.842741 | 0.978212 | −1.02227 |
| ULK2 | NM_014683 | 3.67E−07 | 0.114604 | 1.08802 | 1.08802 |
| DOCK4 | NM_014705 | 1.38E−09 | 0.493932 | 1.041 | 1.041 |
| CSRNP2 | NM_030809 | 0.00012 | 0.359696 | 1.10028 | 1.10028 |
| LOC284033 | AK095052 | 0.00016 | 0.570407 | 0.909486 | −1.09952 |
| DAAM1 | NM_014992 | 6.72E−05 | 0.251365 | 0.851003 | −1.17508 |
| HERC5 | NM_016323 | 8.63E−05 | 0.819443 | 1.0581 | 1.0581 |
| FGD6 | NM_018351 | 5.97E−06 | 0.002279 | 1.70722 | 1.70722 |
| C17orf39 | NM_024052 | 5.60E−05 | 0.161354 | 1.16761 | 1.16761 |
| TIPARP | NM_015508 | 7.04E−06 | 0.107258 | 1.21591 | 1.21591 |
| ADARB1 | NM_001033049 | 0.000101 | 0.777626 | 1.03288 | 1.03288 |
| TLL1 | NM_012464 | 0.000226 | 0.898293 | 1.02117 | 1.02117 |
| EFCAB1 | NM_024593 | 1.04E−07 | 0.950193 | 0.995138 | −1.00489 |
| CAMSAP1L1 | NM_203459 | 6.46E−06 | 0.324162 | 0.857355 | −1.16638 |
| BMPR2 | NM_001204 | 0.000242 | 0.784615 | 1.04425 | 1.04425 |
| CPA4 | NM_016352 | 9.48E−07 | 0.699415 | 1.06872 | 1.06872 |
| UBE2Q2 | NM_173469 | 0.001354 | 0.604512 | 0.921019 | −1.08575 |
| CAB39L | NM_030925 | 1.27E−06 | 0.966734 | 0.996609 | −1.0034 |
| TUBA1A | NM_006009 | 0.07469 | 0.880957 | 0.951587 | −1.05088 |
| ORM2 | NM_000608 | 6.33E−06 | 6.64E−06 | 0.18966 | −5.27259 |
| CLCA2 | NM_006536 | 8.01E−11 | 0.54187 | 0.943932 | −1.0594 |
| NIN | NM_020921 | 4.70E−08 | 0.563194 | 1.05777 | 1.05777 |
| EML1 | NM_001008707 | 2.17E−06 | 0.018105 | 0.781747 | −1.27919 |
| MYO3B | NM_138995 | 0.000197 | 0.693778 | 1.04793 | 1.04793 |
| BBOX1 | NM_003986 | 3.51E−10 | 0.578459 | 0.954801 | −1.04734 |
| ZFP36L1 | NM_004926 | 1.83E−05 | 0.189293 | 1.13282 | 1.13282 |
| KRT17 | NM_000422 | 5.11E−07 | 0.968239 | 0.988188 | −1.01195 |
| EPHA4 | NM_004438 | 6.81E−06 | 0.028544 | 0.731987 | −1.36615 |
| ASAP1 | NM_018482 | 5.27E−05 | 0.908222 | 1.01469 | 1.01469 |
| PARD6G | NM_032510 | 3.74E−05 | 0.442029 | 0.908643 | −1.10054 |
| TUBA4A | NM_006000 | 3.89E−08 | 4.59E−05 | 0.631549 | −1.58341 |
| LOC84740 | NR_026892 | 6.94E−08 | 4.32E−08 | 0.079982 | −12.5028 |
| TMEM40 | NM_018306 | 9.61E−06 | 0.477775 | 1.14474 | 1.14474 |
| ARL14 | NM_025047 | 3.77E−07 | 0.001152 | 0.447393 | −2.23517 |
| BTBD11 | NM_001018072 | 2.57E−06 | 0.6461 | 1.05836 | 1.05836 |
| SPRR1B | NM_003125 | 6.11E−09 | 0.023005 | 0.613446 | −1.63014 |
| HIPK3 | NM_005734 | 0.000546 | 0.455342 | 0.894018 | −1.11855 |
| PLS3 | NM_005032 | 1.43E−05 | 0.369579 | 0.907194 | −1.1023 |
| SULF2 | NM_018837 | 8.04E−05 | 0.978442 | 1.00325 | 1.00325 |
| IGFL2 | NM_001002915 | 1.96E−08 | 0.18195 | 1.20035 | 1.20035 |
| SNAPC1 | NM_003082 | 0.000251 | 0.134936 | 1.25428 | 1.25428 |
| MYO9A | NM_006901 | 0.000169 | 0.6042 | 0.944044 | −1.05927 |
| CASP14 | NM_012114 | 6.22E−07 | 0.74079 | 0.948526 | −1.05427 |
| LOC100131726 | NR_024479 | 7.07E−07 | 0.098444 | 0.787812 | −1.26934 |
| TSHZ3 | NM_020856 | 7.41E−06 | 0.613753 | 0.895285 | −1.11696 |
| FBXO27 | NM_178820 | 5.13E−05 | 0.329511 | 1.11889 | 1.11889 |
| DDX26B | NM_182540 | 2.10E−07 | 0.062861 | 1.11723 | 1.11723 |
| IL1F9 | NM_019618 | 0.005257 | 0.63113 | 1.13872 | 1.13872 |
| CSDA | NM_003651 | 6.66E−05 | 0.05142 | 1.27608 | 1.27608 |
| SLC30A4 | NM_013309 | 7.96E−06 | 0.697786 | 1.03216 | 1.03216 |
| RAB9A | NM_004251 | 0.000183 | 0.912979 | 0.987744 | −1.01241 |
| DSG4 | NM_001134453 | 2.29E−05 | 0.937078 | 1.00917 | 1.00917 |
| MYCBP2 | NM_015057 | 0.000554 | 0.98013 | 0.996926 | −1.00308 |
| STK3 | NM_006281 | 1.09E−05 | 0.098316 | 1.23481 | 1.23481 |
| GABRP | NM_014211 | 0.013059 | 0.008814 | 0.254153 | −3.93463 |
| SLC6A11 | NM_014229 | 1.84E−05 | 0.695102 | 1.0464 | 1.0464 |
| KRT5 | NM_000424 | 3.19E−09 | 0.358413 | 1.1361 | 1.1361 |
| CCL27 | NM_006664 | 0.001975 | 0.873923 | 0.974307 | −1.02637 |
| PTPN14 | NM_005401 | 1.54E−05 | 0.153434 | 1.19364 | 1.19364 |
| C3orf34 | NM_032898 | 1.69E−08 | 0.005592 | 1.29853 | 1.29853 |
| LAYN | NM_178834 | 1.42E−06 | 0.085162 | 1.25227 | 1.25227 |
| NEK1 | NM_012224 | 0.002354 | 0.892793 | 1.02864 | 1.02864 |
| LY6K | NM_017527 | 4.86E−05 | 0.681217 | 1.08069 | 1.08069 |
| ULBP1 | NM_025218 | 0.004888 | 0.991583 | 1.00362 | 1.00362 |
| TMPRSS11F | NM_207407 | 4.77E−06 | 0.854635 | 0.971205 | −1.02965 |
| GADD45A | NM_001924 | 0.00014 | 0.192993 | 1.20764 | 1.20764 |
| PPP1R14C | NM_030949 | 2.91E−05 | 0.329808 | 1.17407 | 1.17407 |
| NAV3 | NM_014903 | 8.29E−06 | 0.896389 | 1.01437 | 1.01437 |
| TFPI2 | NM_006528 | 2.13E−11 | 0.005769 | 1.28215 | 1.28215 |
| SPRR2A | NM_005988 | 3.49E−08 | 0.119661 | 0.84558 | −1.18262 |
| CYYR1 | NM_052954 | 0.000156 | 0.805222 | 1.04086 | 1.04086 |
| AQP3 | NM_004925 | 1.81E−08 | 0.155138 | 0.896981 | −1.11485 |
| SNCA | NM_000345 | 1.06E−07 | 0.1312 | 1.15872 | 1.15872 |
| MORC3 | NM_015358 | 9.63E−06 | 0.477014 | 0.939386 | −1.06453 |
| FAT2 | NM_001447 | 2.37E−07 | 0.679351 | 1.04914 | 1.04914 |
| PKP1 | NM_000299 | 2.05E−07 | 0.622854 | 1.06254 | 1.06254 |
| FEZ1 | NM_005103 | 8.18E−08 | 0.654346 | 0.947736 | −1.05515 |
| SFRP1 | NM_003012 | 3.17E−05 | 0.81695 | 1.0346 | 1.0346 |
| TGM1 | NM_000359 | 3.34E−07 | 0.200733 | 1.17838 | 1.17838 |
| LYST | NM_000081 | 0.006956 | 0.307764 | 0.777922 | −1.28548 |
| HOXC9 | NM_006897 | 8.36E−05 | 0.696871 | 1.06113 | 1.06113 |
| SHC1 | NM_183001 | 3.06E−05 | 0.571669 | 1.05106 | 1.05106 |
| S100A8 | NM_002964 | 5.73E−10 | 0.31527 | 1.17076 | 1.17076 |
| GSDMC | NM_031415 | 3.72E−09 | 0.551774 | 0.945126 | −1.05806 |
| RAB38 | NM_022337 | 5.10E−09 | 0.32646 | 1.07178 | 1.07178 |
| SAA1 | NM_000331 | 0.003802 | 0.279495 | 1.56599 | 1.56599 |
| HERC3 | NM_014606 | 0.000663 | 0.944061 | 0.987332 | −1.01283 |
| FAM127A | NM_001078171 | 1.05E−05 | 0.856793 | 1.03946 | 1.03946 |
| FLRT2 | NM_013231 | 7.17E−08 | 0.369562 | 1.13719 | 1.13719 |
| PPP4R4 | NM_058237 | 0.000209 | 0.672889 | 1.04805 | 1.04805 |
| INTS6 | NM_012141 | 0.0002 | 0.851968 | 1.02499 | 1.02499 |
| CRCT1 | NM_019060 | 0.000408 | 0.723531 | 0.936078 | −1.06829 |
| DNAJB4 | NM_007034 | 0.000509 | 0.276513 | 1.27336 | 1.27336 |
| ZNF750 | NM_024702 | 3.22E−08 | 0.984116 | 0.997799 | −1.00221 |
| HTR7 | NM_019859 | 0.004453 | 0.405738 | 1.1703 | 1.1703 |
| FABP4 | NM_001442 | 0.097674 | 0.831132 | 1.07496 | 1.07496 |
| TNNT2 | NM_000364 | 4.41E−05 | 0.947216 | 1.01332 | 1.01332 |
| FER | NM_005246 | 0.000641 | 0.85206 | 1.03874 | 1.03874 |
| GJB4 | NM_153212 | 0.001835 | 0.345337 | 1.1685 | 1.1685 |
| STARD5 | NM_181900 | 4.65E−06 | 0.563586 | 0.939106 | −1.06484 |
| DUOXA1 | NM_144565 | 0.000575 | 0.792238 | 1.04246 | 1.04246 |
| SERPINB3 | NM_006919 | 2.94E−08 | 0.330814 | 1.22933 | 1.22933 |
| HIAT1 | NM_033055 | 0.01459 | 0.668688 | 0.897529 | −1.11417 |
| MAL | NM_002371 | 7.23E−05 | 4.46E−05 | 0.370195 | −2.70128 |
| MMP9 | NM_004994 | 0.000388 | 0.439033 | 1.14921 | 1.14921 |
| CD86 | NM_175862 | 0.006281 | 0.433459 | 1.17482 | 1.17482 |
| GM2A | NM_000405 | 8.00E−07 | 0.399998 | 1.09767 | 1.09767 |
| NFAT5 | NM_138714 | 3.81E−06 | 0.062289 | 1.17426 | 1.17426 |
| AJAP1 | NM_018836 | 1.05E−05 | 0.606008 | 1.05091 | 1.05091 |
| CNGA1 | NM_001142564 | 0.045943 | 0.228827 | 1.62113 | 1.62113 |
| OSBPL6 | NM_032523 | 6.84E−08 | 0.520953 | 1.06369 | 1.06369 |
| MTSS1 | NM_014751 | 1.35E−08 | 0.174182 | 0.908619 | −1.10057 |
| TRIM23 | NM_001656 | 6.72E−06 | 0.831719 | 0.981013 | −1.01935 |
| COPZ2 | NM_016429 | 0.000202 | 0.098731 | 0.755395 | −1.32381 |
| C20orf114 | NM_033197 | 1.48E−08 | 1.36E−08 | 0.120917 | −8.27015 |
| SGTB | NM_019072 | 8.79E−05 | 0.451941 | 0.920019 | −1.08693 |
| LYPD3 | NM_014400 | 1.84E−07 | 0.418123 | 1.11096 | 1.11096 |
| ALOX15B | NM_001141 | 3.55E−07 | 0.923828 | 1.00908 | 1.00908 |
| SLC6A15 | NM_182767 | 2.71E−08 | 0.858213 | 1.03144 | 1.03144 |
| MARK3 | NM_001128918 | 1.02E−05 | 0.597147 | 1.04207 | 1.04207 |
| BICD2 | NM_001003800 | 6.95E−05 | 0.944173 | 1.01021 | 1.01021 |
| PTHLH | NM_198965 | 5.00E−08 | 0.972772 | 1.00473 | 1.00473 |
| TPRG1 | NM_198485 | 1.20E−06 | 0.573324 | 1.07377 | 1.07377 |
| CYP4F11 | NM_021187 | 5.46E−05 | 0.399317 | 1.17296 | 1.17296 |
| PARP9 | NM_001146106 | 0.011648 | 0.741725 | 1.11143 | 1.11143 |
| ITGA5 | NM_002205 | 0.000201 | 0.499279 | 1.09129 | 1.09129 |
| CTSL1 | NM_001912 | 1.13E−05 | 0.835256 | 1.02708 | 1.02708 |
| SFN | NM_006142 | 7.85E−09 | 0.097008 | 0.910249 | −1.0986 |
| ETNK2 | NM_018208 | 0.000167 | 0.785506 | 1.04582 | 1.04582 |
| SPINK6 | NM_205841 | 1.67E−10 | 0.261146 | 0.869934 | −1.14951 |
| TFAP2A | NM_003220 | 1.99E−07 | 0.001986 | 0.668566 | −1.49574 |
| EMR2 | NM_013447 | 0.00025 | 0.49845 | 1.08566 | 1.08566 |
| CLCA4 | NM_012128 | 1.90E−07 | 0.950937 | 1.01167 | 1.01167 |
| S100A9 | NM_002965 | 4.79E−07 | 0.991769 | 1.00236 | 1.00236 |
| EPGN | NM_001013442 | 2.48E−08 | 0.788791 | 1.04667 | 1.04667 |
| GJB5 | NM_005268 | 1.40E−06 | 0.004949 | 0.552073 | −1.81136 |
| MPZL2 | NM_144765 | 8.50E−07 | 0.137208 | 0.889307 | −1.12447 |
| NOTCH2 | NM_024408 | 2.38E−06 | 0.008463 | 1.28995 | 1.28995 |
| PTPRZ1 | NM_002851 | 1.89E−09 | 0.968372 | 0.995836 | −1.00418 |
| KRT14 | NM_000526 | 7.05E−10 | 0.522617 | 0.919211 | −1.08789 |
| FAP | NM_004460 | 0.000184 | 0.734636 | 1.07441 | 1.07441 |
| SLC39A2 | NM_014579 | 1.02E−06 | 0.940932 | 0.991832 | −1.00824 |
| TMPRSS11E | NM_014058 | 2.32E−05 | 0.874615 | 1.04834 | 1.04834 |
| KCNQ5 | NM_019842 | 0.002317 | 0.822837 | 1.03968 | 1.03968 |
| ARL4D | NM_001661 | 1.68E−05 | 0.378403 | 1.17177 | 1.17177 |
| PTGS2 | NM_000963 | 0.00066 | 0.663103 | 0.896137 | −1.1159 |
| SIM2 | NM_009586 | 1.67E−06 | 2.08E−06 | 0.377098 | −2.65183 |
| CDH13 | NM_001257 | 6.12E−08 | 0.281605 | 1.16533 | 1.16533 |
| RAB37 | NM_175738 | 4.76E−06 | 1.60E−05 | 0.455964 | −2.19315 |
| NUAK1 | NM_014840 | 0.001449 | 0.8209 | 0.965302 | −1.03595 |
| ST6GALNAC2 | NM_006456 | 9.39E−08 | 0.043628 | 0.804164 | −1.24353 |
| NTM | NM_001144058 | 9.37E−05 | 0.11753 | 1.27909 | 1.27909 |
| PTPRE | NM_006504 | 5.05E−07 | 0.045174 | 1.22391 | 1.22391 |
| EMP1 | NM_001423 | 8.78E−06 | 0.16802 | 1.22748 | 1.22748 |
| PLD5 | NM_152666 | 1.99E−05 | 0.353565 | 1.21395 | 1.21395 |
| GBP6 | NM_198460 | 5.06E−05 | 0.861643 | 0.974096 | −1.02659 |
| LAMP2 | NM_002294 | 0.000116 | 0.460181 | 1.12742 | 1.12742 |
| F2R | NM_001992 | 0.000105 | 0.00593 | 1.84825 | 1.84825 |
| PYGL | NM_002863 | 1.53E−08 | 9.55E−05 | 1.86349 | 1.86349 |
| PGLYRP3 | NM_052891 | 0.001406 | 0.493995 | 1.12867 | 1.12867 |
| ORM1 | NM_000607 | 0.000765 | 0.000815 | 0.385427 | −2.59452 |
| LPCAT2 | NM_017839 | 0.00011 | 0.306782 | 0.843871 | −1.18502 |
| HOXC10 | NM_017409 | 7.80E−05 | 0.670233 | 1.07045 | 1.07045 |
| PLA2G4E | NM_001080490 | 4.00E−08 | 0.343476 | 1.0885 | 1.0885 |
| NEBL | NM_006393 | 9.72E−05 | 0.013729 | 0.595382 | −1.67959 |
| PCDH21 | NM_033100 | 5.93E−05 | 0.596516 | 1.06277 | 1.06277 |
| CALB2 | NM_001740 | 0.000121 | 0.946357 | 0.984058 | −1.0162 |
| FSCN1 | NM_003088 | 0.000138 | 0.308164 | 1.13718 | 1.13718 |
| SWAP70 | NM_015055 | 2.00E−07 | 0.495581 | 1.04461 | 1.04461 |
| MARK1 | NM_018650 | 1.32E−07 | 0.571495 | 1.04579 | 1.04579 |
| IGFL1 | NM_198541 | 4.72E−06 | 0.67711 | 0.924306 | −1.08189 |
| KRT77 | NM_175078 | 1.52E−05 | 0.635682 | 1.07023 | 1.07023 |
| ERC1 | NM_178037 | 6.44E−06 | 0.268554 | 1.08836 | 1.08836 |
| GNAL | NM_182978 | 7.49E−05 | 0.538888 | 0.932404 | −1.0725 |
| SERPING1 | NM_000062 | 2.86E−05 | 0.105436 | 1.36026 | 1.36026 |
| ATP12A | NM_001676 | 0.000248 | 0.941634 | 0.986326 | −1.01386 |
| LAMP3 | NM_014398 | 0.028786 | 0.84616 | 0.949612 | −1.05306 |
| FST | NM_006350 | 5.16E−07 | 0.528152 | 0.913803 | −1.09433 |
| DUOX1 | NM_017434 | 5.36E−05 | 0.882588 | 1.01828 | 1.01828 |
| CYP1B1 | NM_000104 | 0.001671 | 0.624845 | 1.10572 | 1.10572 |
| ERCC6 | NM_000124 | 1.12E−08 | 0.551787 | 0.964147 | −1.03719 |
| ABCA12 | NM_173076 | 4.61E−09 | 0.007414 | 0.613091 | −1.63108 |
| ERCC1 | NM_202001 | 4.26E−05 | 0.767693 | 1.03938 | 1.03938 |
| CCDC109B | NM_017918 | 0.002651 | 0.304482 | 0.777883 | −1.28554 |
| TMEM86A | NM_153347 | 5.02E−05 | 0.374703 | 1.11306 | 1.11306 |
| KCTD1 | NM_001142730 | 2.89E−07 | 0.056283 | 0.864729 | −1.15643 |
| FLJ21511 | NM_025087 | 2.01E−08 | 0.517128 | 1.11595 | 1.11595 |
| MSRB3 | NM_001031679 | 0.000156 | 0.27337 | 1.15808 | 1.15808 |
| GATA3 | NM_001002295 | 1.57E−06 | 0.778059 | 1.02773 | 1.02773 |
| ETS1 | NM_001143820 | 3.08E−08 | 0.008698 | 1.2081 | 1.2081 |
| JUP | NM_002230 | 2.79E−06 | 0.649802 | 0.960165 | −1.04149 |
| TAGLN | NM_001001522 | 0.002535 | 0.716696 | 0.934562 | −1.07002 |
| SLC7A1 | NM_003045 | 2.99E−05 | 0.963659 | 0.995601 | −1.00442 |
| QKI | NM_206855 | 0.000221 | 0.586587 | 1.07727 | 1.07727 |
| XG | NM_001141919 | 5.23E−06 | 0.004947 | 0.529385 | −1.88898 |
| FERMT2 | NM_006832 | 7.03E−07 | 0.005317 | 1.52035 | 1.52035 |
| MACF1 | NM_012090 | 3.54E−05 | 0.856051 | 1.02518 | 1.02518 |
| OSMR | NM_003999 | 0.000719 | 0.491391 | 1.12011 | 1.12011 |
| GNA15 | NM_002068 | 2.27E−06 | 0.015346 | 0.645485 | −1.54922 |
| IFNE | NM_176891 | 1.48E−08 | 0.451362 | 0.922917 | −1.08352 |
| AMZ2 | NM_016627 | 5.92E−05 | 0.401675 | 1.09997 | 1.09997 |
| TBC1D19 | NM_018317 | 1.69E−05 | 0.680163 | 0.960578 | −1.04104 |
| CRIM1 | NM_016441 | 4.50E−07 | 0.803268 | 1.02068 | 1.02068 |
| CALML5 | NM_017422 | 2.24E−05 | 0.723728 | 1.05588 | 1.05588 |
| GPR64 | NM_001079858 | 3.59E−05 | 6.66E−05 | 0.32254 | −3.10039 |
| SNX24 | NM_014035 | 0.00317 | 0.671286 | 0.911784 | −1.09675 |
| SERPINB13 | NM_012397 | 2.87E−11 | 0.936936 | 0.99293 | −1.00712 |
| KRT15 | NM_002275 | 1.07E−09 | 0.510567 | 0.933518 | −1.07122 |
| MCC | NM_001085377 | 5.92E−06 | 0.448767 | 1.08813 | 1.08813 |
| TP63 | NM_003722 | 1.98E−09 | 0.410825 | 0.924053 | −1.08219 |
| CYB5R1 | NM_016243 | 8.18E−08 | 0.004949 | 0.723942 | −1.38133 |
| SERPINB2 | NM_001143818 | 0.000522 | 0.711281 | 0.87827 | −1.1386 |
| MARVELD1 | NR_026753 | 0.000246 | 0.009597 | 1.657 | 1.657 |
| ERRFI1 | NM_018948 | 4.24E−05 | 0.017005 | 1.41294 | 1.41294 |
| SLCO3A1 | NM_013272 | 3.37E−06 | 0.028271 | 1.22074 | 1.22074 |
| TIMP1 | NM_003254 | 7.98E−06 | 0.189025 | 0.79233 | −1.2621 |
| CAPRIN2 | NM_001002259 | 0.000102 | 0.152241 | 1.22629 | 1.22629 |
| PLTP | NM_006227 | 0.000998 | 0.31831 | 1.18776 | 1.18776 |
| CALCRL | NM_005795 | 7.23E−07 | 0.001763 | 1.35315 | 1.35315 |
| IFIH1 | NM_022168 | 0.015725 | 0.682984 | 0.882473 | −1.13318 |
| CLIC4 | NM_013943 | 0.001914 | 0.982278 | 1.00382 | 1.00382 |
| IRF6 | NM_006147 | 2.28E−07 | 0.26013 | 1.10495 | 1.10495 |
| A2ML1 | NM_144670 | 7.47E−08 | 0.278771 | 0.773098 | −1.2935 |
| FCHSD2 | NM_014824 | 3.04E−05 | 0.065187 | 0.76483 | −1.30748 |
| DNAJB5 | NM_001135005 | 0.0014 | 0.173331 | 1.32736 | 1.32736 |
| TIAM1 | NM_003253 | 1.18E−06 | 0.077225 | 0.823143 | −1.21486 |
| CAPNS2 | NM_032330 | 1.59E−07 | 0.142157 | 1.40374 | 1.40374 |
| KATNAL1 | NM_001014380 | 1.94E−06 | 0.254881 | 1.16813 | 1.16813 |
| GRHL3 | NM_198173 | 3.64E−09 | 0.965385 | 0.997695 | −1.00231 |
| MAP2 | NM_002374 | 1.28E−07 | 0.775196 | 0.976737 | −1.02382 |
| SMARCA1 | NM_003069 | 3.35E−05 | 0.00418 | 1.61793 | 1.61793 |
| C9orf95 | NR_023352 | 0.00091 | 0.727821 | 1.05715 | 1.05715 |
| LUM | NM_002345 | 0.00038 | 0.001159 | 0.326911 | −3.05894 |
| MLF1 | NM_001130157 | 0.000152 | 0.180647 | 1.22499 | 1.22499 |
| RPE65 | NM_000329 | 0.004304 | 0.170309 | 1.38576 | 1.38576 |
| KLF7 | NM_003709 | 3.34E−07 | 0.530485 | 0.946912 | −1.05606 |
| STEAP4 | NM_024636 | 4.23E−09 | 0.202174 | 1.14781 | 1.14781 |
| ARSJ | NM_024590 | 3.70E−05 | 0.412978 | 1.1068 | 1.1068 |
| FGF5 | NM_004464 | 0.000358 | 0.815346 | 0.950385 | −1.05221 |
| IFI44L | NM_006820 | 0.001777 | 0.615482 | 1.297 | 1.297 |
| TNC | NM_002160 | 3.71E−06 | 0.913931 | 1.01482 | 1.01482 |
| LY6D | NM_003695 | 0.00028 | 0.31527 | 1.19356 | 1.19356 |
| SLITRK6 | NM_032229 | 0.00074 | 0.813976 | 0.942404 | −1.06112 |
| RAET1E | NM_139165 | 3.95E−06 | 0.457886 | 1.11564 | 1.11564 |
| SEC14L2 | NM_012429 | 2.11E−06 | 0.872635 | 0.986661 | −1.01352 |
| DUSP7 | NM_001947 | 3.65E−06 | 0.479135 | 1.05805 | 1.05805 |
| ELK3 | NM_005230 | 1.44E−06 | 0.091065 | 1.21577 | 1.21577 |
| SMURF2 | NM_022739 | 8.79E−06 | 0.160301 | 1.15019 | 1.15019 |
| TRIM29 | NM_012101 | 1.30E−08 | 0.01246 | 0.767939 | −1.30219 |
| UGT1A9 | NM_021027 | 3.69E−06 | 3.62E−06 | 0.278572 | −3.58974 |
| | — | 0.017796 | 0.201567 | 1.62323 | 1.62323 |
| SERPINE1 | NM_000602 | 0.000464 | 0.459577 | 0.803811 | −1.24407 |
| MYO5A | NM_000259 | 6.81E−10 | 0.072884 | 1.15323 | 1.15323 |
| | — | 1.57E−06 | 0.708859 | 0.949462 | −1.05323 |
| EGFR | NM_005228 | 7.76E−08 | 0.207808 | 1.10214 | 1.10214 |
| SLC38A2 | NM_018976 | 7.02E−08 | 0.006881 | 0.783891 | −1.27569 |
| HAS2 | NM_005328 | 0.004297 | 0.749226 | 1.06814 | 1.06814 |
| LRRC8C | NM_032270 | 1.86E−05 | 0.03772 | 1.5262 | 1.5262 |
| MPDZ | NM_003829 | 0.001944 | 0.09554 | 1.49927 | 1.49927 |
| DDX60 | NM_017631 | 0.006426 | 0.31499 | 1.42656 | 1.42656 |
| PCDHB2 | NM_018936 | 0.000695 | 0.00194 | 0.463338 | −2.15825 |
| IL1B | NM_000576 | 5.25E−07 | 0.175488 | 0.795989 | −1.2563 |
| BBS9 | NM_198428 | 0.003029 | 0.779505 | 0.944932 | −1.05828 |
| STEAP1 | NM_012449 | 0.135915 | 0.392934 | 1.65021 | 1.65021 |
| CD274 | NM_014143 | 5.19E−05 | 0.272787 | 0.849893 | −1.17662 |
| SLC39A6 | NM_012319 | 3.85E−07 | 0.295822 | 0.900772 | −1.11016 |
| MGAM | NM_004668 | 1.99E−07 | 2.04E−06 | 0.472744 | −2.11531 |
| SEMA3C | NM_006379 | 0.000153 | 0.339352 | 0.858519 | −1.1648 |
| WDFY2 | NM_052950 | 2.45E−08 | 0.0586 | 1.11927 | 1.11927 |
| LDOC1 | NM_012317 | 1.18E−05 | 0.219631 | 1.1671 | 1.1671 |
| GLTP | NM_016433 | 0.000199 | 0.50547 | 1.10974 | 1.10974 |
| CAPN13 | NM_144575 | 1.06E−07 | 2.33E−06 | 0.454007 | −2.20261 |
| IKZF2 | NM_001079526 | 1.78E−06 | 0.737336 | 0.965418 | −1.03582 |
| RBP1 | NM_001130992 | 1.32E−06 | 2.52E−06 | 6.62115 | 6.62115 |
| SCGB2A1 | NM_002407 | 4.48E−06 | 1.05E−05 | 0.299559 | −3.33824 |
| IGFBP6 | NM_002178 | 6.93E−06 | 0.066023 | 1.40829 | 1.40829 |
| C7orf10 | NM_024728 | 1.87E−07 | 0.003813 | 1.28009 | 1.28009 |
| SLPI | NM_003064 | 1.62E−06 | 0.399929 | 0.86513 | −1.1559 |
| CD109 | NM_133493 | 9.85E−09 | 0.116094 | 0.829 | −1.20627 |
| SP110 | NM_080424 | 0.002794 | 0.887726 | 1.02949 | 1.02949 |
| VGLL1 | NM_016267 | 0.000107 | 0.087514 | 1.52081 | 1.52081 |
| LRP12 | NM_013437 | 1.05E−06 | 0.183473 | 1.14468 | 1.14468 |
| PRB4 | NM_002723 | 0.023507 | 0.587484 | 1.23804 | 1.23804 |
| OPTN | NM_001008211 | 1.79E−05 | 0.563586 | 1.06182 | 1.06182 |
| YPEL5 | NM_001127401 | 0.000254 | 0.896436 | 1.01909 | 1.01909 |
| SULT2B1 | NM_004605 | 7.72E−05 | 0.035154 | 1.39265 | 1.39265 |
| CDH3 | NM_001793 | 7.22E−06 | 0.025863 | 1.33645 | 1.33645 |
| MLLT11 | NM_006818 | 7.84E−05 | 0.116316 | 1.5896 | 1.5896 |
| DRAP1 | NM_006442 | 0.000223 | 0.912902 | 0.984063 | −1.01619 |
| CASP1 | NM_033292 | 1.68E−06 | 0.001887 | 2.02769 | 2.02769 |
| TFAP2C | NM_003222 | 7.94E−06 | 0.262607 | 0.890248 | −1.12328 |
| EREG | NM_001432 | 0.000459 | 0.212032 | 1.49169 | 1.49169 |
| CAV1 | NM_001753 | 3.96E−08 | 0.011415 | 1.49491 | 1.49491 |
| OGFRL1 | NM_024576 | 8.46E−06 | 0.100833 | 1.34002 | 1.34002 |
| DEFB1 | NM_005218 | 1.17E−05 | 0.375581 | 0.818125 | −1.22231 |
| MRAP2 | NM_138409 | 1.35E−07 | 3.63E−06 | 0.356703 | −2.80345 |
| KRT6A | NM_005554 | 9.88E−08 | 0.048042 | 0.615147 | −1.62563 |
| FDXACB1 | NM_138378 | 5.58E−06 | 0.986807 | 0.995518 | −1.0045 |
| PI3 | NM_002638 | 2.91E−05 | 0.001449 | 2.44838 | 2.44838 |
| FZD6 | NM_003506 | 0.00022 | 0.283483 | 1.18043 | 1.18043 |
| SPTLC3 | NM_018327 | 1.08E−05 | 0.024726 | 1.3906 | 1.3906 |
| CLIP4 | NM_024692 | 1.46E−05 | 0.020137 | 1.55802 | 1.55802 |
| RAB31 | NM_006868 | 1.73E−06 | 0.027134 | 1.47898 | 1.47898 |
| KLK13 | NM_015596 | 2.92E−05 | 0.924165 | 1.01351 | 1.01351 |
| CD44 | NM_000610 | 6.59E−06 | 3.45E−05 | 3.47222 | 3.47222 |
| DZIP1 | NM_198968 | 3.02E−06 | 0.000644 | 1.74329 | 1.74329 |
| | — | 0.010603 | 0.8323 | 0.943196 | −1.06023 |
| CALD1 | NM_033138 | 1.59E−05 | 0.016215 | 1.64354 | 1.64354 |
| TUBG2 | NM_016437 | 7.33E−06 | 0.014841 | 1.36075 | 1.36075 |
| PRKCH | NM_006255 | 2.73E−05 | 0.070199 | 1.27231 | 1.27231 |
| KRT16 | NM_005557 | 3.75E−08 | 0.673889 | 0.91816 | −1.08913 |
| FAM63B | NM_001040450 | 1.96E−05 | 0.068358 | 1.42823 | 1.42823 |
| C3orf67 | BC132815 | 3.15E−07 | 0.014819 | 1.24586 | 1.24586 |
| RIMKLB | NM_020734 | 1.58E−05 | 0.708638 | 0.942199 | −1.06135 |
| ATP10D | NM_020453 | 1.04E−06 | 0.710817 | 0.945438 | −1.05771 |
| ARL4C | NM_005737 | 8.07E−07 | 0.021078 | 0.736817 | −1.35719 |
| FRMD6 | NM_001042481 | 5.92E−07 | 0.64632 | 0.936182 | −1.06817 |
| KRT13 | NM_153490 | 2.54E−07 | 0.343476 | 1.22976 | 1.22976 |
| KIF3A | NM_007054 | 0.006094 | 0.875752 | 1.05211 | 1.05211 |
| FBP2 | NM_003837 | 6.19E−06 | 0.000103 | 0.387372 | −2.5815 |
| PHLDB2 | NM_001134438 | 2.38E−06 | 0.800695 | 1.04141 | 1.04141 |
| SNAI2 | NM_003068 | 4.56E−08 | 0.002729 | 2.11752 | 2.11752 |
| IFIT1 | NM_001548 | 0.000118 | 0.197351 | 1.72857 | 1.72857 |
| SCEL | NM_144777 | 7.26E−07 | 0.053756 | 1.43077 | 1.43077 |
| PITPNC1 | NM_181671 | 4.67E−08 | 0.016814 | 1.2283 | 1.2283 |
| DDX58 | NM_014314 | 1.91E−05 | 0.495801 | 1.12876 | 1.12876 |
| ITGBL1 | NM_004791 | 1.75E−05 | 0.000146 | 0.272938 | −3.66384 |
| PYGB | NM_002862 | 7.79E−06 | 0.722711 | 1.03732 | 1.03732 |
| CAV2 | NM_001233 | 2.37E−05 | 0.012178 | 1.67738 | 1.67738 |
| DCBLD2 | NM_080927 | 1.79E−07 | 0.949103 | 0.993914 | −1.00612 |
| PALMD | NM_017734 | 8.09E−09 | 0.007945 | 1.30826 | 1.30826 |
| EPHX3 | NM_024794 | 0.007575 | 0.134248 | 1.6368 | 1.6368 |
| UGT2B15 | NM_001076 | 8.86E−05 | 0.00237 | 0.270879 | −3.69168 |
| CYBRD1 | NM_024843 | 7.67E−07 | 0.102411 | 0.759074 | −1.31739 |
| STXBP1 | NM_003165 | 1.67E−06 | 0.001928 | 1.60414 | 1.60414 |
| IFIT3 | NM_001031683 | 0.012789 | 0.194776 | 1.7348 | 1.7348 |
| PLK2 | NM_006622 | 4.19E−06 | 0.007484 | 1.63934 | 1.63934 |
| ATP2B4 | NM_001001396 | 2.62E−06 | 0.830052 | 0.973585 | −1.02713 |
| MID2 | NM_012216 | 1.44E−07 | 0.026004 | 1.22325 | 1.22325 |
| CCL28 | NM_148672 | 9.94E−05 | 0.001755 | 2.11344 | 2.11344 |
| ZNF185 | NM_007150 | 9.88E−08 | 0.165017 | 1.11833 | 1.11833 |
| USP44 | NM_032147 | 3.67E−05 | 1.70E−05 | 2.35095 | 2.35095 |
| STC2 | NM_003714 | 0.007593 | 0.005833 | 2.20664 | 2.20664 |
| ANXA1 | NM_000700 | 1.81E−05 | 0.105435 | 1.2453 | 1.2453 |
| DAPP1 | NM_014395 | 6.66E−07 | 0.875543 | 1.01624 | 1.01624 |
| TCP11L1 | NM_018393 | 1.16E−07 | 0.181957 | 1.11133 | 1.11133 |
| PIK3C2G | NM_004570 | 1.19E−05 | 0.000104 | 0.280701 | −3.56251 |
| ITGB6 | NM_000888 | 1.44E−05 | 0.597992 | 1.08268 | 1.08268 |
| IFI6 | NM_002038 | 0.000558 | 0.942851 | 1.01746 | 1.01746 |
| AREG | NM_001657 | 9.80E−08 | 0.134643 | 1.22225 | 1.22225 |
| TCEA3 | NM_003196 | 6.03E−05 | 0.002289 | 0.488683 | −2.04632 |
| NKX6-3 | NM_152568 | 0.000222 | 0.000368 | 2.03266 | 2.03266 |
| CRABP2 | NM_001878 | 1.24E−09 | 0.006692 | 0.727076 | −1.37537 |
| NEXN | NM_144573 | 0.000501 | 0.013166 | 2.20289 | 2.20289 |
| HSPC159 | NM_014181 | 7.31E−08 | 0.189829 | 0.89285 | −1.12001 |
| SAMD9L | NM_152703 | 0.002066 | 0.368463 | 1.27406 | 1.27406 |
| TNS4 | NM_032865 | 1.33E−06 | 0.053027 | 1.31768 | 1.31768 |
| PTPN13 | NM_080683 | 2.15E−06 | 0.229783 | 1.27158 | 1.27158 |
| SERPINB7 | NM_003784 | 5.70E−08 | 0.156301 | 1.35763 | 1.35763 |
| PSCA | NM_005672 | 6.84E−07 | 1.03E−05 | 0.232733 | −4.29678 |
| NPSR1 | NM_207172 | 2.73E−06 | 0.000387 | 0.441786 | −2.26354 |
| CTH | NM_001902 | 0.000612 | 0.001894 | 2.32478 | 2.32478 |
| MX1 | NM_001144925 | 0.000642 | 0.098969 | 1.77021 | 1.77021 |
| LRRC6 | NM_012472 | 0.002159 | 0.001201 | 2.68093 | 2.68093 |
| TNFRSF10C | NM_003841 | 7.46E−05 | 4.13E−05 | 3.48054 | 3.48054 |
| CYR61 | NM_001554 | 4.65E−05 | 0.007289 | 1.81334 | 1.81334 |
| CXCL17 | NM_198477 | 1.48E−06 | 2.73E−06 | 0.068137 | −14.6764 |
| ANKRD50 | NM_020337 | 1.33E−05 | 0.430417 | 1.1196 | 1.1196 |
| GSTM4 | NM_000850 | 1.62E−06 | 1.28E−05 | 5.0432 | 5.0432 |
| GSTM2 | NM_000848 | 0.000898 | 0.00096 | 2.63294 | 2.63294 |
| HRASLS2 | NM_017878 | 0.000251 | 0.005555 | 0.333009 | −3.00292 |
| C11orf92 | NM_207429 | 9.79E−08 | 5.84E−05 | 0.439849 | −2.27351 |
| ODAM | NM_017855 | 6.17E−06 | 0.005289 | 2.37484 | 2.37484 |
| AHNAK2 | NM_138420 | 8.07E−08 | 0.121631 | 1.21709 | 1.21709 |
| DDX43 | NM_018665 | 0.000328 | 0.000219 | 4.30453 | 4.30453 |
| IFI16 | NM_005531 | 1.79E−06 | 0.033083 | 0.580452 | −1.7228 |
| SLC16A4 | NM_004696 | 0.000184 | 5.26E−05 | 13.7411 | 13.7411 |
| AK5 | NM_174858 | 0.000101 | 0.000176 | 3.27481 | 3.27481 |
| FKBP5 | NM_001145775 | 8.41E−05 | 0.353899 | 0.788638 | −1.26801 |
| THBS1 | NM_003246 | 6.21E−05 | 0.987229 | 1.00468 | 1.00468 |
| KCNJ15 | NM_002243 | 5.99E−07 | 0.000143 | 2.08393 | 2.08393 |
| LCN2 | NM_005564 | 4.89E−05 | 0.233941 | 1.34648 | 1.34648 |
| HS3ST5 | NM_153612 | 7.36E−05 | 0.000372 | 2.45858 | 2.45858 |
| CAPN9 | NM_006615 | 1.80E−09 | 1.36E−07 | 0.215242 | −4.64593 |
| CLDN10 | NM_182848 | 1.13E−06 | 2.14E−06 | 2.86826 | 2.86826 |
| KLK10 | NM_002776 | 2.17E−06 | 0.339011 | 1.14633 | 1.14633 |
| SAMD9 | NM_017654 | 8.12E−06 | 0.503399 | 0.840268 | −1.1901 |
| HLA-DMB | NM_002118 | 0.000348 | 0.00051 | 3.27169 | 3.27169 |
| KLK7 | NM_139277 | 5.21E−07 | 0.000418 | 0.383008 | −2.61091 |
| NTS | NM_006183 | 0.018973 | 0.025816 | 4.29633 | 4.29633 |
| TGFB2 | NM_001135599 | 0.001966 | 0.007631 | 2.39047 | 2.39047 |
| CYP2E1 | NM_000773 | 3.35E−05 | 0.000411 | 2.1897 | 2.1897 |
| ALDH3A1 | NM_000691 | 1.89E−08 | 2.05E−05 | 0.410963 | −2.43331 |
| CCBE1 | NM_133459 | 4.81E−06 | 1.48E−06 | 4.33119 | 4.33119 |
| MATN2 | NM_002380 | 6.77E−06 | 2.81E−06 | 3.87824 | 3.87824 |
| MFAP5 | NM_003480 | 3.58E−05 | 0.045897 | 0.390572 | −2.56035 |
| BAAT | NM_001701 | 5.52E−08 | 1.78E−06 | 2.7204 | 2.7204 |
| SLC15A1 | NM_005073 | 4.59E−06 | 2.83E−05 | 2.62386 | 2.62386 |
| MXRA5 | NM_015419 | 0.000382 | 0.001087 | 3.33408 | 3.33408 |
| FGF2 | NM_002006 | 4.92E−06 | 7.38E−05 | 2.76247 | 2.76247 |
| IFI44 | NM_006417 | 0.000107 | 0.808255 | 1.10526 | 1.10526 |
| CSTA | NM_005213 | 2.09E−07 | 0.770347 | 1.08448 | 1.08448 |
| SERPINB5 | NM_002639 | 1.66E−09 | 0.001188 | 0.591351 | −1.69104 |
| GPR87 | NM_023915 | 1.16E−07 | 0.008842 | 1.88356 | 1.88356 |
| BICC1 | NM_001080512 | 2.71E−06 | 1.66E−05 | 5.66854 | 5.66854 |
| MSN | NM_002444 | 2.62E−07 | 0.016787 | 1.62807 | 1.62807 |
| GKN1 | NM_019617 | 1.12E−07 | 0.001325 | 0.287564 | −3.47749 |
| GKN2 | NM_182536 | 1.22E−08 | 0.00082 | 0.353334 | −2.83019 |

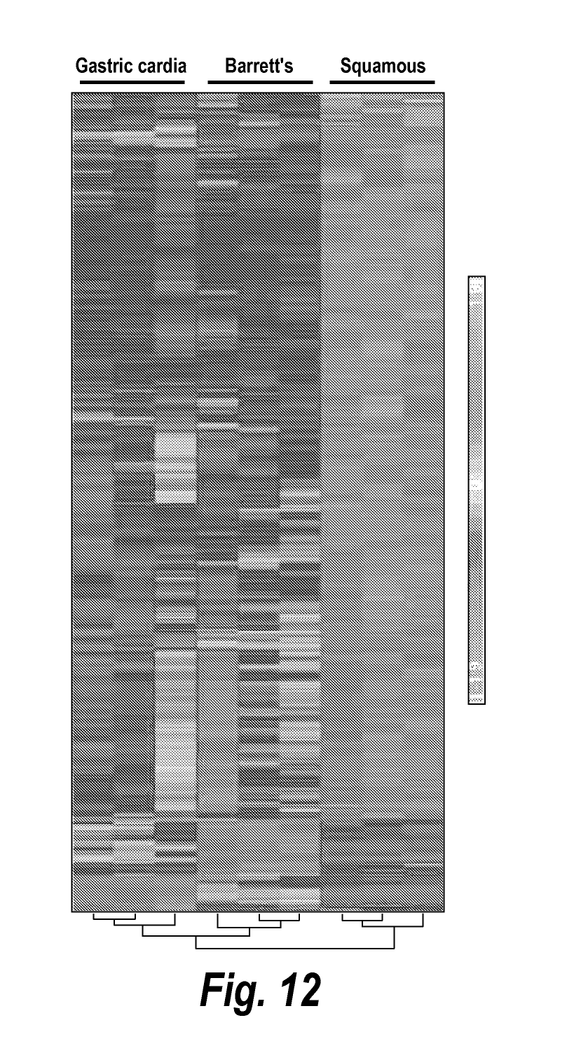

The data in Tables ZZ and YY are also summarized in the heat map shown in FIG. 12.

### Example 7

### Differential Expression of Proteins in Barrett's Esophagus Progenitor Cells Compared to Squamous Progenitor Cells and Gastric Cardia Progenitor Cells

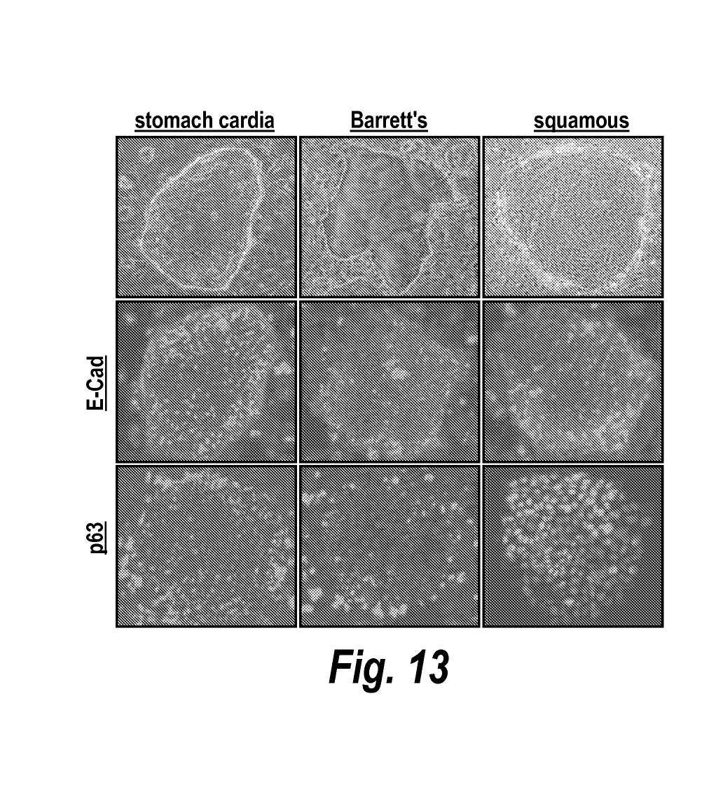

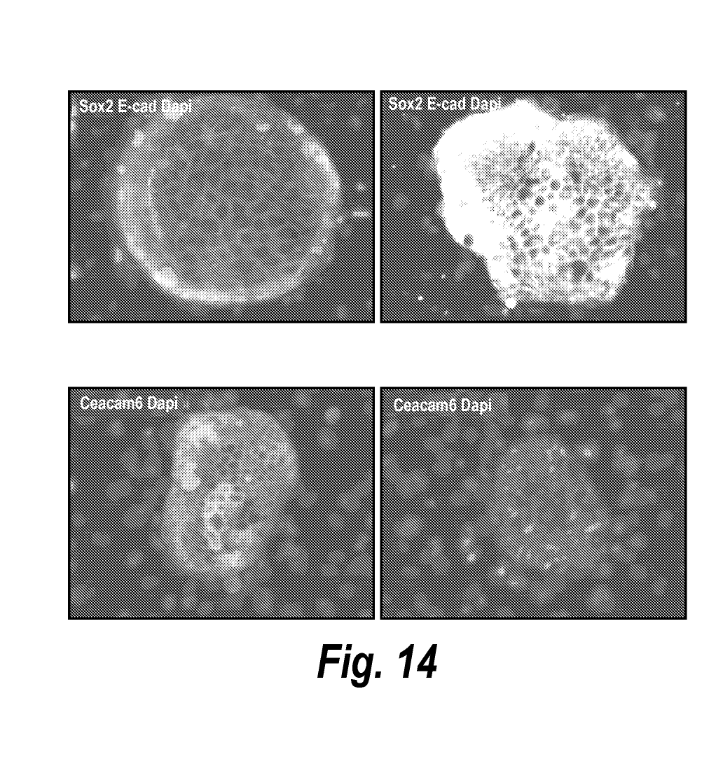

Cultures of Barrett's Esophagus progenitor cells, squamous progenitor cells and gastric cardia progenitor cells were compared to determine expression of p63, CEACAM6 and Sox2. As shown in FIG. 13, p63 is expressed in squamous progenitor cells, but not in gastric cardia or Barrett's progenitor cells. As shown in FIG. 14, Barrett's esophagus progenitor cells (left panels) lack Sox2 while expressing CEAMCAM6, while gastric cardia progenitor cells (right panels) express Sox2, but lack CEAMCAM6.

### EQUIVALENTS

Those skilled in the art will recognize, or be able to ascertain using no more than routine experimentation, many equivalents to the specific embodiments of the invention described herein. Such equivalents are intended to be encompassed by the following claims.

1-55. (canceled)

56. A composition comprising a clonal population of stem cells isolated from an esophagus of a subject, wherein the stem cells differentiate into Barrett's epithelium.

57. The composition of claim 56, wherein the stem cells are characterized as having an mRNA profile wherein the amount of one or more of GSTM4, SLC16A4, CMBL, CEACAM6, NRFA2, CFTR, GCNT3 mRNA are each in the range of 5 to 50 percent of the amount of actin mRNA in the stem cell.

58. The composition of claim 56, wherein the stein cells are characterized as having an mRNA profile wherein the amount of one or more of GSTM4, SLC16A4, CMBL, CEACAM6, NRFA2, CFTR, GCNT3 mRNA are each at least 10 percent of the amount of actin mRNA in the stem cell.

59. The composition of claim 56, wherein the stem cells are further characterized as having an mRNA profile wherein mRNA for BICC1 and NTS are present in detectable levels.

60. The composition of claim 56, wherein the stem cells are further characterized as having an mRNA profile wherein mRNA for SOX2, p63, Krt20, GKN1/2, FABP1/2, Krt14, CXCL17 is present in amounts less than 0.1 percent the level of actin.

61. The composition of claim 56, wherein the stem cells are further characterized as CEACAM6 positive, and Krt20, Sox2 and p63 negative, as detected by standard antibody staining.

62. A method of screening for an agent which may be used to treat or prevent the occurrence of Barrett's esophagus, or which may be effective in the detection of the Barrett's esophagus, comprising
a) providing the cells of claim 1;
b) contacting the BE stem cells with the test agent;
c) detecting the ability of the test agent to reduce viability, growth or differentiation of the BE stem cells, or detecting the ability of the test agent to bind to the BE stem cells;
wherein if the test agent reduces the viability, growth or differentiation of the BE stem cells than the test agent may be effective in the treatment or prevention of Barrett's esophagus, or wherein if the test agent binds to the BE stem cells, the test agent may be an agent effective in the detection of the Barrett's esophagus.

63. The method of claim 62, wherein the test agent is also contacted with normal cells or tissue of the alimentary canal, and the differential ability, if any, of the test agent to reduces the viability, growth or differentiation of the normal cells or tissue is compared to that with the BE stem cells.

64. The method of claim 62, wherein the BE stem cells are human BE stein cells.

65. The method of claim 62, wherein the test agent is selected for further drug development if the test reduces the viability, growth or ability to differentiation of the BE stem cells is reduced by at least 70%.

66. The method of claim wherein the BE stem cells are provided as a clonal population of cells.

67. The method of claim 62, wherein the test agent is small molecule, carbohydrate, peptide or nucleic acid.

68. The method of claim 62, wherein the test agent specifically binds to a cell surface protein on the clonal population of cells.

69. A method for treating or preventing Barrett's esophagus and/or esophageal metaplasia in a subject in need thereof comprising administering to the subject an effective amount of an therapeutic agent that is cytotoxic or cytostatic for Barrett's Esophagus (BE) stein cells in the esophagus of the subject, or inhibits differentiation of the BE stem cells to columnar epithelium.

70. The method claim 69, wherein the subject is a mammal.

71. A composition comprising a clonal population of stem cells isolated from an esophagus or gastric cardia of a subject, wherein the stem cells differentiate into gastric cardia cells.

72. The composition of claim 71, wherein the stem cells are characterized as having an mRNA profile wherein the amount of one or more of CXCL17, CAPN6, PSCA, GKN1, GKN2 or MT1 G mRNA are each in the range of 5 to 50 percent of the amount of actin mRNA in the stem cells.

73. The composition of claims 71, wherein the mRNA profile further comprises a profile wherein the amount of one or more of CXCL17, CAPN6, PSCA, GKN1, GKN2 or MT1 G mRNA are each at least 10 percent of the amount of actin mRNA in the stem cells.

74. The composition of claim 71, fluffier characterized as having an mRNA profile wherein mRNA for CEACAM6, p63, FABP1, FABP2, Krt14 or Krt20 are present in amounts less than 0.1 percent the level of actin.

75. The composition of claim 71, wherein the stem cells are further characterized as CEACAM6 negative as detected by standard antibody staining.
